(19) United States
(12) Patent Application Publication
(10) Pub. No.: US 2024/0254204 A1
(43) Pub. Date: Aug. 1, 2024

(54) PROTEIN ANTIGEN-BINDING MOLECULES

(71) Applicant: National University of Singapore, Singapore (SG)

(72) Inventors: Linfa Wang, Singapore (SG); Wan Ni Chia, Singapore (SG); Chee Wah Tan, Singapore (SG); Feng Zhu, Singapore (SG)

(21) Appl. No.: 18/561,266

(22) PCT Filed: May 15, 2022
(86) PCT No.: PCT/SG2022/050323

§ 371 Date: Nov. 15, 2023

(30) Foreign Application Priority Data
May 15, 2021 (SG) 10202105095U
Jun. 25, 2021 (SG) 10202107013P
Apr. 28, 2022 (SG) 10202204610V

(51) Int. Cl. C07K 16/10; A61P 31/14

(52) U.S. Cl. CPC C07K 16/1003; A61P 31/14; C07K 2317/76

(57) ABSTRACT

The present disclosure provides antigen-binding molecule capable of binding to a sarbecovirus spike protein from two or more different sarbecovirus. Nucleic acids, expression 5 vectors, and cells for making and using the same. In particular antigen-binding molecules such as neutralising antibodies capable of inhibiting interaction between the sarbecovirus spike protein and ACE2, thus behaving as antagonists of infection of ACE2-expressing cells by the sarbecovirus. Antigen-binding molecules described herein are provided with a combination of advantageous properties over known SARS-COV-2 antibodies.

### REFERENCE TO RELATED APPLICATIONS

This application claims the priority to Singapore patent application No. 10202105095U, filed on 15 May 2021; Singapore patent application No. 10202107013P, filed on 25 Jun. 2021; and Singapore patent application No. 10202204610V, filed on 28 Apr. 2022, the contents of which are incorporated herein by reference.

### TECHNICAL FIELD

The present disclosure relates generally to molecules such as protein antigen-binding molecules suitable for use in treatment or prevention of coronaviral infection particularly SARS related beta coronaviruses (sarbecovirus).

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Emerging zoonotic viruses have become a major threat to public health and world economy in the last two decades as the world underwent three major human infectious disease pandemics caused by coronaviruses (CoVs). The three major human infectious disease pandemics caused by coronaviruses (CoVs) include SARS in 2002-2003 caused by SARS-COV (Peiris et al. Nat Med 2004, 10:S88-97), MERS since 2012 (Zaki et al. N Engl J Med 2012, 367:1814-1820) and ongoing COVID-19 caused by SARS-COV-2 (Wang et al. Lancet 2020, 395:470-473). All of them caused devastating economic and human losses globally. For SARS and MERS, we still don't have a licensed treatment or prevention against future infections by these viruses. For COVID-19, the unprecedented speed of vaccine development has resulted in many licensed vaccines for human use (Fauci Science 2021, 372:109).

There are a large number of CoVs present in wildlife reservoir animals, especially in bats. There is a high chance of future outbreaks (SARS3, SARS4, etc) caused by different, but related CoVs. The current classification of CoVs is shown in FIG. 1. Four genera (alpha, beta, delta and gamma) have been identified and the most at-risk viruses for human infection are all from genus Betacoronavirus (Nat Microbiol 2020, 5:536-544), particularly those from the sub-genus Sarbecovirus. Sarbecoviruses are the most transmissible coronaviruses in humans, possibly because they all use the human angiotensinogen converting enzyme 2 (ACE2) receptor as entry into human cells. There are hundreds of strains of SARS-related coronaviruses (SARSr-CoVs) known only to infect non-human species; bats are a major reservoir of many strains of SARS-related coronaviruses. There are two major clades of sarbecoviruses. Clade 1a includes SARS-COV related CoVs (SC2r-COV) and clade 1b contains SARS-COV-2 related CoVs (SC2r-COV). The sarbecoviruses include SARS-COV, SC1r-COV WIV-1, SC1r-CoV RsSHC014, SARS-COV-2, SARS-COV-2 B.1.1.7 (Alpha), SARS-COV-2 B.1.351 (Beta), SARS-COV-2 B1.617.2 (delta), SC2r-CoV GD-1, GX-P5L and SC2r-COV RaTG13. Studies have shown there is limited cross neutralization between SARS-CoV and SARS-COV-2 (Yang R, et al. EBioMedicine 2020, 58:102890).

In pandemic preparedness and responses, there is a need for a multi-prong approach to combat emerging zoonotic viruses which includes vaccines, therapeutic monoclonal antibodies and small molecular drugs. For SARS-COV-2, the first countermeasure commercial product which received FDA Emergency Use Approval (EUA) was therapeutic monoclonal antibodies (mAbs) derived from COVID-19 patients. Both antibody and T-cell immunity are important for controlling viral infections such as SARS-COV-2 or other sarbecoviral infections. In comparison, Neutralizing antibodies (Nabs) are more important to prevent virus entry and hence initial infection whereas T-cell immunity will kick in later in the infection to reduce or control disease progression. Nabs can be induced either through infection or vaccination. Passive immunization using therapeutic mAbs remains an important part of pandemic response and containment as they can play a key role in treating severe patients, especially in the vulnerable populations (such as immunocompromised patients) or preventing onward transmission by ring-fence application in targeted high-risk populations. Unfortunately, the first generation of therapeutic mAbs for COVID-19 has proven to be less or non-effective for the newly emergent variants of concern (VOC).

The recent emergence of SARS-COV-2 variants, the Alpha COVID-19 variant SARS-COV-2 B.1.1.7; the Beta COVID-19 variant SARS-COV-2 B.1.351 also known as 20H/501Y.V2, or 501Y.V2 variant; the Gamma variant P.1., the Delta SARS-COV-2 B.1.617.2; and the Omicron variants SARS-COV-2 B.1.1.529 BA.1 and BA.2 and the observed reduction of immune protection against new variants from vaccines based on the original virus strain raised a new challenge on the need for a broad-spectrum treatment or prevention against all known and future SARS-COV-2 variants. Other coronaviruses are known to be circulating in wildlife reservoirs such as the SC2r-COV GD-1 and SC2r-COV GX-P5L in pangolin and the SC2r-COV RaTG13, SC1r-COV WIV-1 and SC1r-COV RsSHC014 in bats giving rise to a chance of future outbreaks (SARS3, SARS4, etc) caused by different, but related coronaviruses.

Most SARS-COV-2 vaccines that are currently licensed for use in humans were developed against the S protein of the ancestral strain first identified in Wuhan. The emergence and dominance of VOCs have posed a significant threat and challenge as some of them, especially VOC Omicron, have evolved to escape immunity mainly through evasion of NAbs in either infected or vaccinated individuals regardless of the type of vaccines and even in individuals who have received booster vaccinations or hybrid immunity derived from infection and vaccination.

There exists a need to develop a molecule for use in treatment or prevention of human infection caused by sarbecoviruses and alleviate at least one of the aforementioned problems.

### SUMMARY

Protein antigen-binding molecules such as monoclonal antibodies, nucleic acid, expression vector, cell or composition suitable for broad spectrum pan-sarbecovirus for use in treatment or prevention of coronaviral infection caused by sarbecoviruses is envisaged.

Accordingly, an aspect of the invention refers to an antigen-binding molecule which binds to a sarbecovirus spike protein from two or more different sarbecovirus wherein the antigen-binding molecule comprises:

(i) a heavy chain variable (VH) region incorporating the following CDRs:
HC-CDR1 having the amino acid having at least 85% sequence identity to SEQ ID NO: 1 or SEQ ID No. 111
HC-CDR2 having the amino acid having at least 85% sequence identity to SEQ ID NO:2 or SEQ ID No. 112
HC-CDR3 having the amino acid having at least 85% sequence identity to SEQ ID NO:3 or SEQ ID No. 113; and

(ii) a light chain variable (VL) region incorporating the following CDRs:
LC-CDR1 having the amino acid having at least 85% sequence identity to SEQ ID NO: 4 or SEQ ID No. 114
LC-CDR2 having the amino acid having at least 85% sequence identity to SEQ ID NO: 5 or SEQ ID No. 115
LC-CDR3 having the amino acid having at least 85% sequence identity to SEQ ID NO:6 or SEQ ID No. 116.

Another aspect of the invention refers to an antigen-binding molecule which binds to a sarbecovirus spike protein from two or more different sarbecovirus wherein the antigen-binding molecule comprises:

(i) A Heavy Chain Variable (VH) Region Incorporating the Following CDRs:
HC-CDR1 having the amino acid having formula I: X₁-X₂-X₃-Φ-X₄-Xₙ₁-X₅-X₆:
   wherein X₁ is selected from one of G and E;
   X₂ is selected from one of F, Y, N, G, D, and V;
   X₃ is selected from one of P, T, S, I and F;
   Φ is selected from one of F, V, L, and I;
   X₄ is selected from one of S, T, R, N, G, L, and I;
   Xₙ₁ is selected from one of S, SN, N, M, H, T, G, P, G and D;
   X₅ is selected from one of Y, S, N, I and H;
   X₆ is selected from one of Y, G, W, E, A, N, and T;
HC-CDR2 having the amino acid formula II: X₇-X₈-X₉-Xₙ₂-π-Xₙ₃-X₁₀:
   wherein X₇ is selected from one of I and T;
   X₈ is selected from one of Y, S, N, G, A and T;
   X₉ is selected from one of S, F, P, N, H, I, Y, G, and T;
   Xₙ₂ is selected from one of G, YN, DD, T, NG, DG, S, SS, D, ST, and NT;
   π is selected from one of G, S, P, A and E;
   Xₙ₃ is selected from one of S, I, D, G, F, N, RT, L, and RN;
   X₁₀ is selected from one of T, R, M, K, S, and P;
HC-CDR3 having the amino acid having formula III: Ψ-ζ₁-Xₙ₄-X₁₁-Xₙ₅-X₁₂-X₁₃-X₁₄-ζ₂-X₁₅;
   wherein Ψ is selected from one of A and V,
   ζ₁ is selected from one of R, T, K and L-N
   Xₙ₄ is selected from one of E, HLGGG, GGG, LDIII, DSI, GEAG, RVAIF, LQNG, VTYTS, ADIV, DSLA, DSL, AISQQ, DYYDN, DPL, EGIQG, and DGG;
   X₁₁ is selected from one of L, S, Y, T, A, N, V, and W;
   Xₙ₅ is selected from one of R, S, LET, P, SAT, MATIWV, DGY, SY, PLPF, GS, VV, SVT, FDS, GYYY, EGAAS, V, and QLPY;
   X₁₂ is selected from one of H, W, G, P, T, S, N, and Y;
   X₁₃ is selected from one of Y, P, A, L, S, F, I, V, and G;
   X₁₄ is selected from one of F, I, N, Y, L, and M;
   ζ₂ is selected from one of D, E, G, and S;
   X₁₅ is selected from one of Y, S, L, N, H, C, V, and F;
   and

(ii) a light chain variable (VL) region incorporating the following CDRs:
LC-CDR1 having the amino acid having formula IV: X₁₆-X₁₇-X₁₈-Xₙ₆-ζ₃-X₁₉:
   wherein X₁₆ is selected from one of Q and Y;
   X₁₇ is selected from one of G, S, T, N, I, and A;
   X₁₈ is selected from one of V, I, T, F and L;
   Xₙ₆ is selected from one of S, G, N, V, R, LYSSNNK, LYRSNNK, LQNNGY, VQSNGY, VHSDGN, MQLNGY and SS;
   ζ₃ is selected from one of S, N, and T;
   X₁₉ is selected from one of W, Y, S and N;
LC-CDR2 having the amino acid having formula V: X₂₀-X₂₁-S:
   wherein X₂₀ is selected from one of A, W, K, T, G, L, and D;
   X₂₁ is selected from one of A, S, G, I, and T
LC-CDR3 having the amino acid having formula VI: X₂₂-ζ₄-X₂₃-Xₙ₇-ζ₅-X₂₄-X₂₅-Xₙ₈-ζ₆:
   wherein X₂₂ is selected from one of Q, H, and M;
   ζ₄ is selected from one of Q and H;
   X₂₃ is selected from one of Y, S, G, A, L, and T;
   Xₙ₇ is selected from one of F, Y, N, S, L, G, T, YR, and YI;
   ζ₅ is selected from one of S, T, N, Q, and D;
   X₂₄ is selected from one of S, Y, T, D, H, F, P, W, and I;
   X₂₅ is selected from one of P, I, and R;
   Xₙ₈ is selected from one of F, W, K, G, Y, R, P, L, PY, EY, ED, GY, QY, and QI;
   ζ₆ is selected from one of T and S.

According to another aspect there is a nucleic acid, or a plurality of nucleic acids, optionally isolated, encoding the antigen-binding molecule as discussed herein above.

According to another aspect there is an expression vector, or a plurality of expression vectors, comprising a nucleic acid or a plurality of nucleic acids as discussed herein above.

According to another aspect there is a method for producing an antigen-binding molecule which binds to a sarbecovirus spike protein from two or more different sarbecovirus, comprising culturing a cell capable of expressing the antigen binding molecule as discussed herein above under conditions suitable for expression of an antigen-binding molecule by the cell.

According to another aspect there is a composition comprising an antigen-binding molecule as discussed herein above, a nucleic acid or a plurality of nucleic acids as discussed herein above, an expression vector or a plurality of expression vectors as discussed herein above, and a pharmaceutically acceptable carrier, diluent, excipient or adjuvant.

According to another aspect there is an antigen-binding molecule as discussed herein above, a nucleic acid or a plurality of nucleic acids as discussed herein above, an expression vector or a plurality of expression vectors as discussed herein above, or a composition as discussed herein above, for use in treatment or prevention of a disease caused by infection with a sarbecovirus.

According to another aspect there is use of an antigen-binding molecule as discussed herein above, a nucleic acid or a plurality of nucleic acids as discussed herein above, an expression vector or a plurality of expression vectors as discussed herein above, or a composition as discussed herein above, in the manufacture of a medicament for use in treatment or prevention of a disease caused by infection with a sarbecovirus.

According to another aspect there is a method of treating or preventing a disease caused by infection with a sarbecovirus, comprising administering to a subject a therapeutically or prophylactically effective amount of an antigen-binding molecule as discussed herein above, a nucleic acid or a plurality of nucleic acids as discussed herein above, an expression vector or a plurality of expression vectors as discussed herein above, or a composition as discussed herein above.

According to another aspect there is use of an antigen-binding molecule as discussed herein above to inhibit infection of ACE2-expressing cells by a sarbecovirus.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of non-limiting examples only, embodiments of the present invention,

FIG. 1: Family tree of four known coronavirus genera.

FIG. 2: A) Multiplex sVNT on the Luminex platform. B) As shown, all six RBD proteins are able to bind the hACE2 receptor molecular as expected in the following order (from high to low affinity): SARS-COV-2 B.1.351>SARS-COV-2 B.1.1.7=SC2r-COV GX-P5L (pangolin)>SARS-COV-2>SARS-COV>SC2r-COV RaTG13 (bat).

FIG. 3: Multiplex sVNT analysis from five panels of human sera (indicated above) against ten different sarbecoviruses. All sera were used at 1:20. A cut-off of 30% was set as previously determined. A cut-off of 30% was set as previously determined.

FIG. 4: Titration of NAbs expressed in NT50 using the same serum panels and viruses as in A. Samples were tested at dilutions from 1:20 to 1:20,480 using a 4-fold serial titration. A cut-off of 1:100 was set as previously determined.

FIG. 5: Converting the non-dominant cross-neutralizing antibody responses to immune dominant ones by cross-immunization with genetically distant sarbecovirus antigen(s)

FIG. 6: Inhibition of pan-sarbecovirus mAb-RBD interaction by different human serum panels. (A) Inhibition of RBD-ACE2 interaction by rabbit mAb 5D7B7 which has a pan-sarbecovirus RBD binding ability. (B) Inhibition of 5B7D7 binding to different RBDs by the five serum panels as indicated. Scatter plot shows all data points and the 0th, 25th, 50th, 75th, and 100th percentile are marked. Statistical significance was determined using Wilcoxon signed-rank test. The “SARS-Vaccinated” was set as the reference group for comparison.

FIG. 7: Staining of B-cells with fluorescently labeled RBDs. (A) Representative flow cytometry plots for SARS-Vaccinated group (n=5), Healthy-Vaccinated group (n=6) and COVID-19-Vaccinated group (n=5) showing frequency of SARS-COV-1- and SARS-COV-2-double positive cells. (B) Scatter plot on frequency of SARS-COV-1 and SARS-COV-2 double positive cells of all SARS-COV-1 or SARS-COV-2-positive cells. Scatter plot shows all data points and the 0th, 25th, 50th, 75th, and 100th percentile are marked. Statistical significance was determined using Wilcoxon signed-rank test. P value was indicated on top of each plot. n.s indicates not significant, with P>0.05.

FIG. 8: Neutralization patterns from rabbit hyper immune sera targeting different beta coronavirus RBD proteins. A cut-off of 1:100 was set as previously determined.

FIG. 9: Schematic diagram to sort out double positive B-cells which produce antibodies binding to RBDs from both viruses.

FIG. 10: Phylogenetic analysis and sequence alignment of ACE2-binding sarbecovirus RBDs. (A) Phylogenetic tree based on receptor binding domain (RBD) sequences of sarbecoviruses which can bind human ACE2. The tree was generated using PhyML with general-time-reversible (GTR) substitution model and 1,000 bootstrap replicates. Numbers at the branches are percentage bootstrap values for the associated nodes. Scale bar represents the number of substitutions per site. Sarbecovirus RBDs involved in this study were bolded. (B) RBD amino acid sequence identity percentage among different sarbecoviruses in decreasing order for SARS-COV-1 (right to left) and SARS-COV-2 (left to right). (C) Alignment of amino acid sequences of sarbecovirus RBDs used in this study. Red indicates mutation/deletion. Amino acids critical for SARS-COV-2 RBD-ACE2 interaction are indicated by the blue dots above. Viruses in the SARS-COV-1 clade are shaded in grey.

FIG. 11: CD19+B cells were sorted for those positive for binding to SARS-COV-1 (SC1+) and SARS-COV-2 (SC2+) RBD tetramers.

FIG. 12: Data for the top six monoclonal antibodies identified in the preliminary screen and four control monoclonal antibodies using multiplex sVNT platform.

FIG. 13: 50% inhibitory concentration (IC50; ng/ml) of monoclonal antibodies in blocking RBD-ACE2 engagement using multiplex surrogate virus neutralization competition format.

FIG. 14: Ability of monoclonal antibodies to neutralize different sarbecoviruses against eight spike-pseudotyped reporter viruses which included SARS-COV-2 ancestral and four VOCs (Alpha, Delta, Beta and Gamma), two zoonotic sarbecoviruses (GX-P5L and WIV-1), and SARS-COV-1.

FIG. 15: Results of monoclonal antibody neutralisation tests against ancestral SARS-COV-2, Omicron BA.1 and Omicron BA.2 viruses, using three different platforms A multiplex sVNT, B pseudovirus neutralization test (pVNT) and C plaque reduction neutralization test (PRNT).

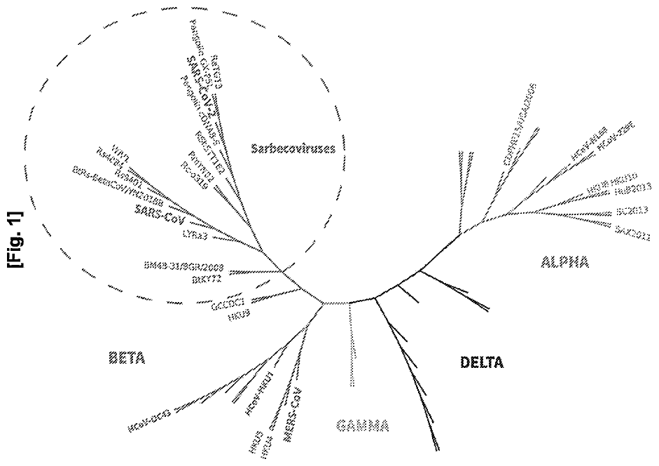

### DETAILED DESCRIPTION

The present disclosure provides an antigen-binding molecule(s) capable of binding to a sarbecovirus spike protein from two or more different sarbecovirus, in particular neutralising antibodies capable of inhibiting the interaction between the sarbecovirus spike protein and ACE2, thus behaving as antagonists of infection of ACE2-expressing cells by a range of sarbecovirus. Antigen-binding molecules described herein are provided with a combination of advantageous properties over known SARS-COV-2 antibodies.

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the document, unless the context requires otherwise, the word “include” or variations such as “includes” or “including” will be understood to imply the inclusion of a stated integer or group of integers but not the exclusion of any other integer or group of integers.

Unless defined otherwise, all other technical and scientific terms used herein have the same meaning as is commonly understood by a skilled person to which the subject matter herein belongs.

According to various embodiments there is an antigen-binding molecule which binds to a sarbecovirus spike protein from two or more different sarbecoviruses wherein the antigen-binding molecule comprises:

(i) a heavy chain variable (VH) region incorporating the following CDRs:
HC-CDR1 having the amino acid having at least 85% sequence identity to SEQ ID NO: 1 or SEQ ID NO:111
HC-CDR2 having the amino acid having at least 85% sequence identity to SEQ ID NO:2 or SEQ ID NO:112
HC-CDR3 having the amino acid having at least 85% sequence identity to SEQ ID NO:3 or SEQ ID NO:113; and

(ii) a light chain variable (VL) region incorporating the following CDRs:
LC-CDR1 having the amino acid having at least 85% sequence identity to SEQ ID NO: 4 or SEQ ID NO:114
LC-CDR2 having the amino acid having at least 85% sequence identity to SEQ ID NO: 5 or SEQ ID NO:115
LC-CDR3 having the amino acid having at least 85% sequence identity to SEQ ID NO:6 or SEQ ID NO:116.

Throughout the description, it is to be appreciated that the term “antigen-binding molecule” and its plural form refers to one or more molecule which is capable of binding to a target antigen, and encompasses monoclonal antibodies, polyclonal antibodies, monospecific and multispecific antibodies (e.g., bispecific antibodies), and antibody fragments (e.g. Fv, scFv, Fab, scFab, F(ab′)2, Fab2, diabodies, triabodies, scFv-Fc, minibodies, single domain antibodies (e.g. VhH), etc.), as long as they display binding to the relevant target molecule(s) and block entry into the cell via ACE2.

In various embodiments, two or more different sarbecovirus may include an antigen-binding molecule being capable of binding to the spike protein of 2, or 3, or, 4, or 5 or 6 or 7 or 8 or 9 or 10 or more different sarbecovirus. In various embodiments, for example, the antigen-binding molecule is capable of binding to SARS-COV spike protein, and which is also capable of binding to SARS-COV-2 spike protein. In various embodiments, the antigen-binding molecule is capable of binding multiple sarbecovirus spike proteins. For example, the antigen-binding molecule is capable of binding to SARS-COV spike protein; and/or capable of binding to SARS-COV-2 spike protein and or capable of binding to SARS-COV-2 Alpha, and/or capable of binding to SARS-COV-2 Beta and/or capable of binding to SARS-CoV-2 Delta and/or capable of binding to SC2r-COV RaTG13, and/or capable of binding to SC2r-COV GX-P5L and/or capable of binding to SC2r-COV GD-1 and/or capable of binding to any other sarbecovirus spike protein such as SC2r-CoVRmYN02; RacCS203 or future unknown sarbecoviruses. A broad-spectrum antigen-binding molecule has the advantage of being able to block most sarbecoviruses effectively assisting in preventing infection of both known and unknown sarbecoviruses.

In various embodiments, the term capable of binding may comprise inhibition or neutralization of 30% or more binding between the sarbecovirus spike protein and ACE2. In various embodiments, inhibition or neutralization of 30% or more binding between the sarbecovirus spike protein and ACE2 may be selected from one of at least 30%, 35%, 40%, 41%, 42%, 43%, 44%, 45%, 49%, 50%, 51%, 52%, 53%, 54%, 55%, 56%, 57%, 58%, 59%, 60%,61%, 62%, 63%, 64%, 65%, 66%, 67%, 68%, 69%, 70%, 75%, 80% or greater inhibition or neutralisation. In various embodiments the antigen-binding molecule binds to the sarbecovirus spike protein from two or more different sarbecovirus comprising inhibition or neutralization of 30% or more binding between the sarbecovirus spike protein and ACE2 (e.g. one of at least 30%, 35%, 40%, 41%, 42%, 43%, 44%, 45%, 49%, 50%, 51%, 52%, 53%, 54%, 55%, 56%, 57%, 58%, 59%, 60%,61%, 62%, 63%, 64%, 65%, 66%, 67%, 68%, 69%, 70%, 75%, 80% or greater inhibition or neutralisation).

In various embodiments, the antigen-binding molecule comprises polyclonal antibodies isolated from a patient that has had SARS and recovered. In various embodiments, the antigen-binding molecule comprises polyclonal antibodies isolated from a patient that has had SARS and recovered and has been vaccinated with a COVID-19 vaccination. For example, the polyclonal antigen-binding molecule is capable of binding to SARS-COV spike protein, and which is also capable of binding to SARS-COV-2 spike protein. In various embodiments, the polyclonal antigen-binding molecule is capable of binding multiple sarbecovirus spike proteins. For example, the polyclonal antigen-binding molecule is capable of binding to SARS-COV spike protein; and/or capable of binding to SARS-COV-2 spike protein and or capable of binding to SARS-COV-2 Alpha, and/or capable of binding to SARS-COV-2 Beta and/or capable of binding to SARS-COV-2 Delta and/or capable of binding to SC2r-COV RaTG13, and/or capable of binding to SC2r-COV GX-P5L and/or capable of binding to SC2r-COV GD-1 and/or capable of binding to any other sarbecovirus spike protein such as SC2r-CoVRmYN02; RacCS203 or future unknown sarbecoviruses. A broad-spectrum antigen-binding molecule has the advantage of being able to block most sarbecoviruses effectively assisting in preventing infection of both known and unknown sarbecoviruses.

In various embodiments, the antigen-binding molecule which binds to a sarbecovirus spike protein from two or more different sarbecovirus wherein the antigen-binding molecule comprises:

(i) a heavy chain variable (VH) region incorporating the following CDRs:
HC-CDR1 having the amino acid having formula I: X₁-X₂-X₃-Φ-X₄-Xₙ₁-X₅-X₆:
   wherein X₁ is selected from one of amino acids G and E; X₂ is selected from one of amino acids F, Y, N, G, D, and V; X₃ is selected from one of amino acids P, T, S, I and F; Φ is selected from one of hydrophobic amino acids F, V, L, or I; X₄ is selected from one of amino acids S, T, R, N, G, L, and I; Xₙ₁ is selected from one of amino acid sequence S, SN, N, M, H, T, G, P, G and D; X₅ is selected from one of amino acids Y, S, N, I and H; X₆ is selected from one of amino acids Y, G, W, E, A, N, and T;
HC-CDR2 having the amino acid formula II: X₇-X₈-X₉-Xₙ₂-π-Xₙ₃-X₁₀.
   wherein X₇ is selected from one of amino acids I and T; X₈ is selected from one of amino acids Y, S, N, G, A and T; X₉ is selected from one of amino acids S, F, P, N, H, I, Y, G, and T; Xₙ₂ is selected from one of amino acid sequence G, YN, DD, T, NG, DG, S, SS, D, ST, and NT; π is selected from one of small amino acids G, S, P, A and E; Xₙ₃ is selected from one of amino acid sequence S, I, D, G, F, N, RT, L, and RN; X₁₀ is selected from one of amino acids T, R, M, K, S, and P;
HC-CDR3 having the amino acid having formula III: Ψ-ζ₁-Xₙ₄-X₁₁-Xₙ₅-X₁₂-X₁₃-X₁₄-ζ₂-X₁₅;
   wherein Ψ is selected from one of aliphatic amino acids A and V, ζ₁ is selected from one of hydrophilic amino acids R, T, K and L-N; Xₙ₄ is selected from one of amino acid sequence E, HLGGG, GGG, LDIII, DSI, GEAG, RVAIF, LQNG, VTYTS, ADIV, DSLA, DSL, AISQQ, DYYDN, DPL, EGIQG, and DGG; X₁₁ is selected from one of amino acids L, S, Y, T, A, N, V, and W; Xₙ₅ is selected from one of amino acid sequence R, S, LET, P, SAT, MATIWV, DGY, SY, PLPF, GS, VV, SVT, FDS, GYYY, EGAAS, V, and QLPY; X₁₂ is selected from one of amino acids H, W, G, P, T, S, N, and Y; X₁₃ is selected from one of amino acids Y, P, A, L, S, F, I, V, and G; X₁₄ is selected from one of amino acids F, I, N, Y, L, and M; ζ₂ is selected from one of hydrophilic amino acids D, E, G, and S; X₁₆ is selected from one of amino acids Y, S, L, N, H, C, V, and F; and

(ii) a light chain variable (VL) region incorporating the following CDRs:
LC-CDR1 having the amino acid having formula IV: X₁₆-X₁₇-X₁₈-Xₙ₆-ζ₃-X₁₉:
   wherein X₁₆ is selected from one of amino acids Q and Y; X₁₇ is selected from one of amino acids G, S, T, N, I. and A; X₁₈ is selected from one of amino acids V, I, T, F and L; Xₙ₆ is selected from one of amino acid sequence S, G, N, V, R, LYSSNNK, LYRSNNK, LQNNGY, VQSNGY, VHSDGN, MQLNGY and SS; ζ₃ is selected from one of hydrophilic amino acids S, N, and T; X₁₉ is selected from one of amino acids W, Y, S and N;
LC-CDR2 having the amino acid having formula V: X₂₀-X₂₁-S:
   wherein X₂₀ is selected from one of amino acids A, W, K, T, G, L, and D; X₂₁ is selected from one of amino acids A, S, G, I, and T
LC-CDR3 having the amino acid having formula VI: X₂₂-ζ₄-X₂₃-Xₙ₇-ζ₅-X₂₄-X₂₅-X₁₈-ζ₆:
   wherein X₂₂ is selected from one of amino acids Q, H, and M; ζ₄ is selected from one of hydrophilic amino acids Q and H; X₂₃ is selected from one of amino acids Y, S, G, A, L, and T; Xₙ₇ is selected from one of amino acid sequence F, Y, N, S, L, G, T, YR, and YI; ζ₅ is selected from one of hydrophilic S, T, N, Q, and D; X₂₄ is selected from one of amino acids S, Y, T, D, H, F, P, W, and I; X₂₅ is selected from one of amino acids P, I, and R; Xₙ₈ is selected from one of amino acid sequence F, W, K, G, Y, R, P, L, PY, EY, ED, GY, QY, and QI; ζ₆ is selected from one of hydrophilic amino acids T and S.

The antigen-binding molecule with CDRs falling within these formulas demonstrated neutralizing across several sarbecovirus including SARS-COV-2. Antibodies 1 (SS6V1-B5) to 20 (SS6V20-F5) are examples of antigen-binding molecules with CDRs falling within these formulas. These are some of best cross-clade Neutralising antibodies reported to date.

In various embodiments, the formulas are the same as those listed above with the exception that Φ of formula I is selected from one of hydrophobic amino acids F, L, or I; X₅ of formula I is selected from one of Y, S, I and H; X₆ of formula I is selected from one of Y, W, E, A, N, and T; X₈ of formula Il is I; Xₙ₂ of formula Il is selected from one of DD, T, NG, DG, S, SS, D, ST, and NT; ζ₁ of formula III is selected from one of hydrophilic amino acids R, T, and K; Xₙ₄ of formula Ill is selected from one of HLGGG, GGG, LDIII, DSI, GEAG, LQNG, VTYTS, ADIV, DSLA, DSL, AISQQ, DYYDN, DPL, EGIQG, and DGG; X₁₁ of formula III is selected from one of S, Y, T, A, V, and W; Xₙ₅ of formula III is selected from one of S, LET, P, SAT, MATIWV, SY, PLPF, GS, VV, SVT, FDS, GYYY, EGAAS, V, and QLPY; X₁₂ of formula III is selected from one of W, G, P, T, S, N, and Y; Xₙ₆ of formula IV is selected from one of S, G, N, V, R, LYRSNNK, LQNNGY, VQSNGY, VHSDGN, MQLNGY and SS; X₂₃ of formula VI is selected from one of Y, S, G, A, and T; Xₙ₈ of formula VI is selected from one of F, W, K, G, Y, R, P, L, EY, ED, GY, QY, and QI. The antigen-binding molecule with CDRs falling within these formulas demonstrated pan-sarbecovirus neutralizing across a large breadth of sarbecovirus including SARS-COV-1 and SARS-COV-2. Antigen-binding molecule with CDRs falling within these formulas were double positive for staining by both SARS-COV-1 and SARS-COV-2 RBD proteins. Antibody 1 (SS6V1-B5) and antibodies 4 to 20 (SS6V4-A1, SS6V5-C3 . . . to SS6V20-F5) are examples of antigen-binding molecules with CDRs falling within these formulas.

In various embodiments, wherein X₁ is G; X₂ is selected from one of G, F, Y and V; X₃ is selected from one of S, I, T and F; Φ is selected from one of F, L and I; X₄ is selected from one of R, S, G, L, T and I; Xₙ₁ is selected from one of P, N, T, D and G; X₅ is selected from one of Y, S and H; X₆ is selected from one of E, N and Y; X₇ is I; X₈ is selected from one of G, S, N and Y; X₉ is selected from one of I, N, S, T and F; Xₙ₂ is selected from one of T, S, SS and NT; IT is selected from one of G, E, S and A; Xₙ₃ is selected from one of G, S, F, I and N; X₁₀ is selected from one of T, M and P; Ψ is A; ζ₁ is R; Xₙ₄ is selected from one of VTYTS, GGG; DYYDN, and DGG; X₁₁ is selected from one of S, Y and W; X₁₅ is selected from one of PLPF, LET, GYYY and QLPY; X₁₂ is selected from one of W, Y and G; X₁₃ is selected from one of F, P, G, and Y; X₁₄ is selected from one of F, M and L; ζ₂ is selected from one of D and E; X₁₅ is selected from one of Y, L, V, F and S; X₁₆ is selected from one of Q and Y; X₁₇ is selected from one of G and S; X₁₈ is selected from one of I, F and L; Xₙ₆ is selected from one of G, LQNNGY, R, VQSNGY, S and MQLNGY; ζ₃ is selected from one of N, S and T; X₁₉ is selected from one of Y or S; X₂₀ is selected from one of A, L and G; X₂₁ is selected from one of A, S, T and G; X₂₂ is selected from one of Q, M and L; ζ₄ is Q; X₂₃ is selected from one of T, S, Y and G; Xₙ₇ is selected from one of YR, L, and Y; ζ₅ is selected from one of T, Q, and S; X₂₄ is selected from one of P, I, Wand T; X₂₅ is P; Xₙ₈ is selected from one of ED, G, QI and L; ζ₆ is selected from one of S and T. That is in various embodiments formula I comprises GX₂X₃ΦX₄X₅X₆X₇ wherein X₂ is selected from one of G, F, Y and V; X₃ is selected from one of S, I, T and F; Φ is selected from one of F, L and I; X₄ is selected from one of R, S, G, L, T and I; X₅ is selected from one of P, N, T, D and G; X₆ is selected from one of Y, S and H; X₇ is selected from one of E, N and Y; Formula II comprises IX₉X₁₀Xₙ₁πXₙ₂X₁₁ wherein X₉ is selected from one of G, S, N and Y; X₁₀ is selected from one of I, N, S, T and F; Xₙ₁ is selected from one of S, SS and NT; π is selected from one of G, E, S and A; Xₙ₂ is selected from one of G, S, F, I and N; X₁₁ is selected from one of T, M and P; formula III comprises ARXₙ₃X₁₂Xₙ₄X₁₃X₁₄X₁₅ζ₂X₁₆ wherein X₁₃ is selected from one of VTYTS, GGG; DYYDN, and DGG; X₁₂ is selected from one of S, Y and W; Xₙ₄ is selected from one of PLPF, LET, GYYY and QLPY; X₁₃ is selected from one of W, Y and G; X₁₄ is selected from one of F, P, G, and Y; X₁₅ is selected from one of F, M and L; ζ₂ is selected from one of D and E; X₁₆ is selected from one of Y, L, V, F and S; formula IV comprises X₁₇X₁₈X₁₉Xₙ₅ζ₃Ω wherein X₁₇ is selected from one of aromatic amino acids Q and Y; X₁₈ is selected from one of G and S; X₁₉ is selected from one of I, F and L; Xₙ₅ is selected from one of G, LQNNGY, R, VQSNGY, S and MQLNGY; ζ₃ is selected from one of N, S and T; Ω is selected from one of Y or S; formula V comprises X₂₀X₂₁S wherein X₂₀ is selected from one of A, L and G; X₂₁ is selected from one of A, S, T and G; and formula VI comprises X₂₂QX₂₃Xₙ₆ζ₅X₂₅PXₙ₇ζ₆ wherein X₂₂ is selected from one of Q, L, M and L; X₂₃ is selected from one of T, S, Y and G; Xₙ₆ is selected from one of YR, L, and Y; ζ₅ is selected from one of T, Q, and S; X₂₅ is selected from one of P, I, W and T; Xₙ₇ is selected from one of ED, G, QI and L; ζ₆ is selected from one of S and T. The antigen-binding molecule with CDRs falling within these formulas demonstrated pan-sarbecovirus neutralizing potency and breadth across most of the sarbecovirus including SARS-COV-1 and SARS-COV-2. Antibody 1 (SS6V1-B5), antibody 11 (SS6V11-E7), antibody 12 (SS6V12-E11), antibody 13 (SS6V13-F1), antibody 19 (SS6V19-F4) and antibody 20 (SS6V20-F5) are examples of antigen-binding molecules with CDRs falling within these formulas.

In various embodiments, the heavy chain variable (VH) region incorporates the following CDRs: HC-CDR1 selected from one of amino acid sequences GFILRNYE, GGFIGPHY, GFTFSTYN, GVSILGSY, GYTFTDYN and GGSIIGYY; HC-CDR2 selected from one of amino acid sequences IGNTGGT, IYISGST, ISSSSSFM, IYFSENT, INTNTGIP and IYFSANT; HC-CDR3 selected from one of amino acid sequences ARVTYTSSPLPFWFLDL, ARGGGYLETGPFEY, ARDYYDNSGYYYYGMDV, ARGGGYLETGPFDS, ARDGGWQLPYWYFDL and ARGGGYLETGPLDF; and the light chain variable (VL) region incorporates the following CDRs: LC-CDR1 selected from one of amino acid sequences QSIGNY, QSLLONNGYNY, QSIRTY, QGLVQSNGYNY, YSFSSS and QSLMOLNGYNY; LC-CDR2 selected from one of amino acid sequences AAS, LSS, GTS and LGS; LC-CDR3 selected from one of amino acid sequences QQTYRTPPEDS, MQSLQIPGT, LQTYSTPQIT, MQGLQTPGT, QQYYSWPLT and MQGLQIPGT.

In various embodiments, wherein X₁ is G; X₂ is selected from one of G and V; X₃ is selected from one of S, and F; Φ is I; X₄ is selected from one of G, L and I; Xₙ₁ is selected from one of P and G; X₅ is selected from one of Y, S and H; X₆ is Y; X₇ is I; X₈ is Y; X₉ is selected from one of I and F; Xₙ₂ is S; π is selected from one of G, E and A; Xₙ₃ is selected from one of S and N; X₁₀ is T; Ψ is A; ζ₁ is R; Xₙ₄ is GGG; X₁₁ is Y; Xₙ₅ is LET; X₁₂ is G; X₁₃ is P; X₁₄ is selected from one of F and L; ζ₂ is selected from one of D, and E; X₁₅ is selected from one of Y, F and S; X₁₆ is Q; X₁₇ is selected from one of G and S; X₁₈ is L; Xₙ₆ is selected from one of LQNNGY, VQSNGY and MQLNGY; ζ₃ is N; X₁₉ is Y; X₂₀ is L; X₂₁ is selected from one of S and G; X₂₂ is M; ζ₄ is Q; X₂₃ is selected from one of S and G; Xₙ₇ is L; ζ₅ is Q; X₂₄ is selected from one of I and T; X₂₅ is P; Xₙ₈ is G; ζ₆ is T. That is in various embodiments formula I comprises GX₂X₃₁X₄X₅X₆Y wherein X₂ is selected from one of G and V; X₃ is selected from one of S, and F; X₄ is selected from one of G, L and I; X₅ is selected from one of P and G; and X₆ is selected from one of Y, S and H; formula II comprises IYX₁₀SπXₙ₂T wherein X₁₀ is selected from one of I and F; π is selected from one of G, E and A; Xₙ₂ is selected from one of S and N; formula III comprises ARGGGYLETGPX₁₅ζ₂X₁₆ wherein X₁₅ is selected from one of F and L; ζ₂ is selected from one of D and E; X₁₆ is selected from one of Y, F and S; formula IV comprises QX₁₈LXₙ₅NY wherein X₁₈ is selected from one of G and S; X₁₅ is selected from one of LQNNGY, VQSNGY and MQLNGY; formula V comprises LX₂₁S wherein X₂₁ is selected from one of S and G; and formula VI comprises MQX₂₃LQX₂₅PGT wherein X₂₃ is selected from one of S and G; X₂₅ is selected from one of I and T. The antigen-binding molecule with CDRs falling within these formulas demonstrated pan-sarbecovirus neutralizing potency and breadth of all sarbecovirus tested including SARS-COV-1 and SARS-COV-2. Antibody 11 (SS6V11-E7), antibody 13 (SS6V13-F1) and antibody 20 (SS6V20-F5) are examples of antigen-binding molecules with CDRs falling within these formulas. These antibodies demonstrated the highest potency reported. The three monoclonal antibodies falling in this group maintained their strong neutralization capability across most SARS-COV-2 VOCs and VOIs and Clade-1a sarbecoviruses in different virus neutralization assay platforms. All three antibodies utilized a unique combination of heavy and light chain gene classes exhibiting similarity of more than 90% in their heavy and light chain sequences. These sequences have not been previously reported for sarbecovirus-specific antibodies.

In various embodiments, the heavy chain variable (VH) region incorporates the following CDRs: HC-CDR1 selected from one of amino acid sequences GGFIGPHY, GVSILGSY and GGSIIGYY; HC-CDR2 selected from one of amino acid sequences IYISGST, IYFSENT and IYFSANT; HC-CDR3 selected from one of amino acid sequences ARGGGYLETGPFEY, ARGGGYLETGPFDS and ARGGGYLETGPLDF; and the light chain variable (VL) region incorporates the following CDRs: LC-CDR1 selected from one of amino acid sequences QSLLONNGYNY, QGLVQSNGYNY and QSLMQLNGYNY; LC-CDR2 selected from one of amino acid sequences LSS and LGS; LC-CDR3 selected from one of amino acid sequences MQSLQIPGT, MQGLQTPGT and MQGLQIPGT.

In various embodiments, wherein X₁ is G; X₂ is G; X₃ is selected from one of S, and F; Φ is I; X₄ is selected from one of G, and I; Xₙ₁ is selected from one of P and G; X₅ is selected from one of Y and H; X₆ is Y; X₇ is I; X₈ is Y; X₉ is selected from one of I and F; Xₙ₂ is S; π is selected from one of G and A; Xₙ₃ is selected from one of S and N; X₁₀ is T; Ψ is A; ζ₁ is R; Xₙ₄ is GGG; X₁₇ is Y; Xₙ₅ is LET; X₁₂ is G; X₁₃ is P; X₁₄ is selected from one of F and L; ζ₂ is selected from one of D, and E; X₁₅ is selected from one of Y and F; X₁₆ is Q; X₁₇ is S; X₁₈ is L; Xₙ₆ is selected from one of LQNNGY, and MQLNGY; ζ₃ is N; X₁₉ is Y; X₂₀ is L; X₂₁ is selected from one of S and G; X₂₂ is M; ζ₄ is Q; X₂₃ is selected from one of S and G; Xₙ₇ is L; ζ₅ is Q; X₂₄ is I; X₂₅ is P; Xₙ₈ is G; ζ₆ is T. That is in various embodiments formula I comprises GGX₃IX₄X₅X₆Y wherein X₃ is selected from one of S, and F; X₄ is selected from one of G, and I; X₅ is selected from one of P and G; and X₆ is selected from one of Y and H; formula II comprises IYX₁₀SπXₙ₂T wherein X₁₀ is selected from one of I and F; π is selected from one of G and A; Xₙ₂ is selected from one of S and N; formula III comprises ARGGGYLETGPX₁₅ζ₂X₁₆ wherein X₁₅ is selected from one of F and L; X₁₆ is selected from one of Y and F ζ₂ is selected from one of D and E; formula IV comprises QSLXₙ₅NY wherein Xₙ₅ is selected from one of LQNNGY, and MQLNGY; formula V comprises LX₂₁S wherein X₂₁ is selected from one of S and G; and formula VI comprises MQX₂₃LQIPGT wherein X₂₃ is selected from one of S and G. The antigen-binding molecule with CDRs falling within these formulas demonstrated the best pan-sarbecovirus neutralizing potency and breadth compared to any other antibody reported to date. Epitope mapping studies of antibodies falling within these formulas indicate the antibodies have a unique contact footprint in the RBD. Antibody 11 (SS6V11-E7) and antibody 20 (SS6V20-F5) are examples of antigen-binding molecules with CDRs falling within these formulas.

In various embodiments, the heavy chain variable (VH) region incorporates the following CDRs: HC-CDR1 selected from one of amino acid sequences GGFIGPHY and GGSIIGYY; HC-CDR2 selected from one of amino acid sequences IYISGST and IYFSANT; HC-CDR3 selected from one of amino acid sequences ARGGGYLETGPFEY and ARGGGYLETGPLDF; and the light chain variable (VL) region incorporates the following CDRs: LC-CDR1 selected from one of amino acid sequences QSLLQNNGYNY and QSLMOLNGYNY; LC-CDR2 selected from one of amino acid sequences LSS and LGS; LC-CDR3 selected from one of amino acid sequences MQSLQIPGT and MQGLQIPGT.

In various embodiments, the heavy chain variable (VH) region incorporates the following CDRs: HC-CDR1 having the amino acid sequence GFILRNYE; HC-CDR2 having the amino acid sequence IGNTGGT; HC-CDR3 having the amino acid sequence ARVTYTSSPLPFWFLDL; and the light chain variable (VL) region incorporates the following CDRs: LC-CDR1 having the amino acid sequence QSIGNY; LC-CDR2 having the amino acid sequence AAS; LC-CDR3 having the amino acid sequence QQTYRTPPEDS. In various embodiments these CDRs are formed in heavy chain SEQ ID NO 71 and light chain SEQ ID NO 72 of antibody 1 (SS6V1-B5). In various embodiments these are CDRs are formed in other antigen binding scaffolds listed below provided it is able to bind to a sarbecovirus spike protein from two or more different sarbecovirus.

the heavy chain variable (VH) region incorporates the following CDRs: HC-CDR1 having the amino acid sequence GFTVSSNY; HC-CDR2 having the amino acid sequence IYSGGST; HC-CDR3 having the amino acid sequence ARELRHYFDY; and the light chain variable (VL) region incorporates the following CDRs: LC-CDR1 having the amino acid sequence QGISSY; LC-CDR2 having the amino acid sequence AAS; LC-CDR3 having the amino acid sequence QQLNSYPPYS. In various embodiments these CDRs are formed in heavy chain SEQ ID NO 73 and light chain SEQ ID NO 74 of antibody 2 (SS6V2-G1). In various embodiments these CDRs are formed in other antigen binding scaffolds listed below provided it is able to bind to a sarbecovirus spike protein from two or more different sarbecovirus. This antibody was cloned from SC2+ single positive B cells and while it demonstrates reactivity to SARS-COV-2 RBD it showed minimal reactivity to SARS-COV-1 RBD.

The heavy chain variable (VH) region incorporates the following CDRs: HC-CDR1 having the amino acid sequence GYSFTNSG; HC-CDR2 having the amino acid sequence TNFYNGIT; HC-CDR3 having the amino acid sequence ALNRVAIFNDGYNPLGY; and the light chain variable (VL) region incorporates the following CDRs: LC-CDR1 having the amino acid sequence QSVLYSSNNKNY; LC-CDR2 having the amino acid sequence WAS; LC-CDR3 having the amino acid sequence QQYFSSPFS. In various embodiments these CDRs are formed in heavy chain SEQ ID NO 75 and light chain SEQ ID NO 76 of antibody 3 (SS6V3-G2). In various embodiments these are CDRs are formed in other antigen binding scaffolds listed below provided it is able to bind to a sarbecovirus spike protein from two or more different sarbecovirus. This antibody was cloned from SC2+single positive B cells and while it demonstrates reactivity to SARS-COV-2 RBD it showed minimal reactivity to SARS-CoV-1 RBD.

the heavy chain variable (VH) region incorporates the following CDRs: HC-CDR1 having the amino acid sequence GYTFSMYW; HC-CDR2 having the amino acid sequence IYPDDSDR; HC-CDR3 having the amino acid sequence ARLQNGYSYGLLEN; and the light chain variable (VL) region incorporates the following CDRs: LC-CDR1 having the amino acid sequence QSTLYRSNNKNY; LC-CDR2 having the amino acid sequence WAS; LC-CDR3 having the amino acid sequence QQYYSYPWT. In various embodiments these CDRs are formed in heavy chain SEQ ID NO 77 and light chain SEQ ID NO 78 of antibody 4 (SS6V4-A1). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GYTFTHYW; HC-CDR2 having the amino acid sequence IYPDDSDT; HC-CDR3 having the amino acid sequence ATADIVVGSNFFDH; and the light chain variable (VL) region incorporates the following CDRs: LC-CDR1 having the amino acid sequence QSISTW; LC-CDR2 having the amino acid sequence KAS; LC-CDR3 having the amino acid sequence QHYNSYIKT. In various embodiments these CDRs are formed in heavy chain SEQ ID NO 79 and light chain SEQ ID NO 80 of antibody 5 (SS6V5-C3). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GFTFNTYA; HC-CDR2 having the amino acid sequence ISSNGGIT; HC-CDR3 having the amino acid sequence VKDSLATVVTLLSY; and the light chain variable (VL) region incorporates the following CDRs: LC-CDR1 having the amino acid sequence QTISSY; LC-CDR2 having the amino acid sequence AAS; LC-CDR3 having the amino acid sequence QQSYSTPGT. In various embodiments these CDRs are formed in heavy chain SEQ ID NO 81 and light chain SEQ ID NO 82 of antibody 6 (SS6V6-C4). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence ENIFSGYW; HC-CDR2 having the amino acid sequence IYPDDSDT; HC-CDR3 having the amino acid sequence ARHLGGGSSWPIDY; and the light chain variable (VL) region incorporates the following CDRs: LC-CDR1 having the amino acid sequence QGISNY; LC-CDR2 having the amino acid sequence AAS; LC-CDR3 having the amino acid sequence QQYSSYPFT. In various embodiments these CDRs are formed in heavy chain SEQ ID NO 83 and light chain SEQ ID NO 84 of antibody 7 (SS6V7-C5). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GFTFSTYA; HC-CDR2 having the amino acid sequence IASDGGIT; HC-CDR3 having the amino acid sequence VKDSLTSVTTIFDC; and the light chain variable (VL) region incorporates the following CDRs: LC-CDR1 having the amino acid sequence QNINSY; LC-CDR2 having the amino acid sequence TAS; LC-CDR3 having the amino acid sequence QQSYTDPYT. In various embodiments these CDRs are formed in heavy chain SEQ ID NO 85 and light chain SEQ ID NO 86 of antibody 8 (SS6V8-D3). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GGSISSNIW; HC-CDR2 having the amino acid sequence IYHSGST; HC-CDR3 having the amino acid sequence ARAISQQYFDSSVLGY; and the light chain variable (VL) region incorporates the following CDRs: LC-CDR1 having the amino acid sequence QSVVTN; LC-CDR2 having the amino acid sequence GAS; LC-CDR3 having the amino acid sequence QQYNNWPGYT. In various embodiments these CDRs are formed in heavy chain SEQ ID NO 87 and light chain SEQ ID NO 88 of antibody 9 (SS6V9-D11). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence EDSFTGYW; HC-CDR2 having the amino acid sequence IYPDDGDT; HC-CDR3 having the amino acid sequence ARHLGGGSSWPIDS; and the light chain variable (VL) region incorporates the following CDRs: LC-CDR1 having the amino acid sequence QGIRNY; LC-CDR2 having the amino acid sequence AAS; LC-CDR3 having the amino acid sequence QQYNNHPFT. In various embodiments these CDRs are formed in heavy chain SEQ ID NO 89 and light chain SEQ ID NO 90 of antibody 10 (SS6V10-E1). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GGFIGPHY; HC-CDR2 having the amino acid sequence IYISGST; HC-CDR3 having the amino acid sequence ARGGGYLETGPFEY; and the light chain variable (VL) region incorporates the following CDRs: LC-CDR1 having the amino acid sequence QSLLONNGYNY; LC-CDR2 having the amino acid sequence LSS; LC-CDR3 having the amino acid sequence MQSLQIPGT. In various embodiments these are CDRs are formed in heavy chain SEQ ID NO 91 and light chain SEQ ID NO 92 of antibody 11 (SS6V11-E7). In various embodiments these CDRs are formed in other antigen binding scaffolds listed below provided it is able to bind to a sarbecovirus spike protein from two or more different sarbecovirus. The antibodies with these CDR's demonstrated one of the best pan-sarbecovirus neutralizing potency and breadth compared to any other antibody reported to date including being the only antibody with neutralization capacity against Omicron BA.2.

The heavy chain variable (VH) region incorporates the following CDRs: HC-CDR1 having the amino acid sequence GFTFSTYN; HC-CDR2 having the amino acid sequence ISSSSSFM; HC-CDR3 having the amino acid sequence ARDYYDNSGYYYYGMDV; and the light chain variable (VL) region incorporates the following CDRs: LC-CDR1 having the amino acid sequence QSIRTY; LC-CDR2 having the amino acid sequence AAS; LC-CDR3 having the amino acid sequence LQTYSTPQIT. In various embodiments these are CDRs are formed in heavy chain SEQ ID NO 93 and light chain SEQ ID NO 94 of antibody 12 (SS6V12-E11). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GVSILGSY; HC-CDR2 having the amino acid sequence IYFSENT; HC-CDR3 having the amino acid sequence ARGGGYLETGPFDS; and the light chain variable (VL) region incorporates the following CDRs: LC-CDR1 having the amino acid sequence QGLVQSNGYNY; LC-CDR2 having the amino acid sequence LGS; LC-CDR3 having the amino acid sequence MQGLQTPGT. In various embodiments these CDRs are formed in heavy chain SEQ ID NO 95 and light chain SEQ ID NO 96 of antibody 13 (SS6V13-F1). In various embodiments these CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GGPISSYY; HC-CDR2 having the amino acid sequence IYYSGST; HC-CDR3 having the amino acid sequence ARDPLAEGAASSGFDN; and the light chain variable (VL) region incorporates the following CDRs: LC-CDR1 having the amino acid sequence QSISSY; LC-CDR2 having the amino acid sequence AAS; LC-CDR3 having the amino acid sequence QQSYTTPRT. In various embodiments these CDRs are formed in heavy chain SEQ ID NO 97 and light chain SEQ ID NO 98 of antibody 14 (SS6V14-F2). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GFTFSSYA; HC-CDR2 having the amino acid sequence ISYDGRTK; HC-CDR3 having the amino acid sequence ARLDIIITPPANDY; and the light chain variable (VL) region incorporates the following CDRs: LC-CDR1 having the amino acid sequence QIVSSNY; LC-CDR2 having the amino acid sequence DAS; LC-CDR3 having the amino acid sequence HQYGDSRRT. In various embodiments these CDRs are formed in heavy chain SEQ ID NO 99 and light chain SEQ ID NO 100 of antibody 15 (SS6V15-F6). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence EFTFSRYT; HC-CDR2 having the amino acid sequence IGGSTPLS; HC-CDR3 having the amino acid sequence ARDSIASATTLFDL; and the light chain variable (VL) region incorporates the following CDRs: LC-CDR1 having the amino acid sequence QAISSY; LC-CDR2 having the amino acid sequence AAS; LC-CDR3 having the amino acid sequence QQSYITPPEYS. In various embodiments these CDRs are formed in heavy chain SEQ ID NO 101 and light chain SEQ ID NO 102 of antibody 16 (L8N16-C7). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GFTFSSYA; HC-CDR2 having the amino acid sequence ISYDGRNK; HC-CDR3 having the amino acid sequence ARGEAGTMATIWVSSYDY; and the light chain variable (VL) region incorporates the following CDRs: LC-CDR1 having the amino acid sequence QSLVHSDGNTY; LC-CDR2 having the amino acid sequence KIS; LC-CDR3 having the amino acid sequence MQATQFPPT. In various embodiments these CDRs are formed in heavy chain SEQ ID NO 103 and light chain SEQ ID NO 104 of antibody 17 (L8N17-G3). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GFTFSSYA; HC-CDR2 having the amino acid sequence ITSNGGGT; HC-CDR3 having the amino acid sequence AREGIQGWVTYFDY; and the light chain variable (VL) region incorporates the following CDRs: LC-CDR1 having the amino acid sequence QSISTN; LC-CDR2 having the amino acid sequence AAS; LC-CDR3 having the amino acid sequence QQTYTTPQYS. In various embodiments these CDRs are formed in heavy chain SEQ ID NO 105 and light chain SEQ ID NO 106 of antibody 18 (SS6V18-E3). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GYTFTDYN; HC-CDR2 having the amino acid sequence INTNTGIP; HC-CDR3 having the amino acid sequence ARDGGWQLPYWYFDL; and the light chain variable (VL) region incorporates the following CDRs: LC-CDR1 having the amino acid sequence YSFSSS; LC-CDR2 having the amino acid sequence GTS; LC-CDR3 having the amino acid sequence QQYYSWPLT. In various embodiments these CDRs are formed in heavy chain SEQ ID NO 107 and light chain SEQ ID NO 108 of antibody 19 (SS6V19-F4). In various embodiments these are CDRs are formed in other antigen binding scaffolds listed below provided it is able to bind to a sarbecovirus spike protein from two or more different sarbecovirus.

The heavy chain variable (VH) region incorporates the following CDRs: HC-CDR1 having the amino acid sequence GGSIIGYY; HC-CDR2 having the amino acid sequence IYFSANT; HC-CDR3 having the amino acid sequence ARGGGYLETGPLDF; and the light chain variable (VL) region incorporates the following CDRs: LC-CDR1 having the amino acid sequence QSLMOLNGYNY; LC-CDR2 having the amino acid sequence LGS; LC-CDR3 having the amino acid sequence MQGLQIPGT. In various embodiments these CDRs are formed in heavy chain SEQ ID NO 109 and light chain SEQ ID NO 110 of antibody 20 (SS6V20-F5). In various embodiments these are CDRs are formed in other antigen binding scaffolds listed below provided it is able to bind to a sarbecovirus spike protein from two or more different sarbecovirus. The antibodies with these CDR's demonstrated one of the best pan-sarbecovirus neutralizing potency and breadth compared to any other antibody reported to date.

In various embodiments, the antigen-binding molecule comprises a polyclonal antigen-binding molecule. In various embodiments, the antigen-binding molecule comprises at least two different antigen-binding domains (i.e. at least two antigen-binding domains, e.g. comprising non-identical VHs and VLs). The higher neutralization potencies of the disclosed antibodies will enable lower dosages of the antigen-binding molecule to be used clinically as individual antigen-binding molecule or mixed in a cocktail of two or more antigen-binding molecule or an antigen-binding molecule with two or mor different antigen-binding domains.

In various embodiments, the antigen-binding molecule binds to two different sarbecovirus spike proteins, such as SARS-COV spike protein and SARS-COV-2 spike protein, and so is at least bispecific. The term “bispecific” means that the antigen-binding molecule is able to bind specifically to at least two distinct antigenic determinants.

In various embodiments, the bispecific antigen-binding molecule may comprise antigen-binding molecules capable of binding to the targets for which the antigen-binding molecule is specific. For example, an antigen-binding molecule which is capable of binding to SARS-COV spike protein, and which is capable of binding to SARS-COV-2 spike protein may comprise a component which is capable of binding to SARS-COV spike protein, and a second component which is capable of binding to SARS-COV-2 spike protein.

In various embodiments, the antigen-binding molecule according to the present disclosure comprises a multispecific antigen-binding molecule that may comprise antigen-binding polypeptides or antigen-binding polypeptide complexes capable of binding to the targets for which the antigen-binding molecule is specific. In some embodiments, a component antigen-binding molecule of a larger antigen-binding molecule may be referred to as an “antigen-binding domain” or “antigen-binding region” of the larger antigen-binding molecule.

In various embodiments, the multispecific antigen-binding molecule is capable of binding to multiple sarbecovirus spike proteins. For example, the multispecific antigen-binding molecule is capable of binding to SARS-COV spike protein; and/or capable of binding to SARS-COV-2 spike protein and or capable of binding to SARS-COV-2 Alpha, and/or capable of binding to SARS-COV-2 Beta and/or capable of binding to SARS-COV-2 Dealta and/or capable of binding to SC2r-COV RaTG13, and/or capable of binding to SC2r-COV GX-P5L and/or capable of binding to SC2r-COV GD-1 and/or capable of binding to any other sarbecovirus spike protein such as SC2r-CoVRmYN02; RacCS203 or future unknown sarbecoviruses. A broad-spectrum antigen-binding molecule has the advantage of being able to block most sarbecoviruses effectively assisting in preventing infection of both known and unknown sarbecoviruses.

Throughout the description, it is to be appreciated that the term ‘Sarbecovirus’ and its plural form include any beta coronavirus that uses angiotensinogen converting enzyme 2 (ACE2) receptor as entry into cells. In various embodiments, the sarbecovirus comprises any beta coronavirus that uses ACE2 receptor as entry into cells. In various embodiments, the sarbecovirus comprises any beta coronavirus that uses human ACE2 receptor as entry into human cells. In various embodiments, the sarbecovirus comprises any known or new sarbecovirus. In various embodiments, the sarbecovirus is selected from the group comprising or consisting of SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GX-P5L, SC1r-CoV Rs2018B, SC1r-COV LYRa11, SC1r-COV WIV-1, SARS-COV, SC2r-COV WIV-1, SC1r-COV RsSHC014, SARS-COV-2 Alpha, SARS-COV-2 Beta, SARS-CoV-2 Delta, SC2r-COV RaTG13, SC2r-COV GD-1 and SC2r-COV GX-P5L.

Throughout the description, it is to be appreciated that the term ‘SARS-COV’ refers to the SARSr-COV having the nucleotide sequence of GenBank: NC_004718.3 (“Severe acute respiratory syndrome coronavirus isolate, complete genome”), and encompasses variants thereof having a nucleotide sequence with at least 85% sequence identity (e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: NC_004718.3 set forth in SEQ ID NO: 7.

Throughout the description, it is to be appreciated that the term ‘SARS-COV-2’ refers to the SARSr-COV having the nucleotide sequence of GenBank: NC_045512.2 (“Severe acute respiratory syndrome coronavirus 2 isolate Wuhan-Hu 1, complete genome”), reported in Zhou et al., Nature (2020) 579: 270-273, and encompasses variants thereof having a nucleotide sequence with at least 85% sequence identity (e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: NC_045512.2 set forth in SEQ ID NO: 8.

Sarbecoviruses like all coronaviruses have a genome which encodes four major structural proteins: the spike (S) protein, the envelope (E) protein, the membrane (M) protein, and the nucleocapsid (N) protein. Generally, the spike protein has a section that incorporates the receptor binding domain (RBD).

In various embodiments, the sarbecovirus spike protein may be characterised by any one of the consensus amino acid sequences set forth in SEQ ID NOS: 18 to 25 (SEQ ID NO: 18; SEQ ID NO: 19; SEQ ID NO: 20; SEQ ID NO: 21; SEQ ID NO: 22; SEQ ID NO: 23; SEQ ID NO: 24; or SEQ ID NO: 25).

Fragments of sarbecovirus spike protein may have a minimum length of one of 25, 50, 100, 200, 300, 400, 500, 600, 700, 800, 900, 1,000, 1,100 or 1,200 amino acids, and may have a maximum length of one of 50, 100, 200, 300, 400, 500, 600, 700, 800, 900, 1,000, 1,100 or 1,200 amino acids.

In various embodiments, the spike protein of SARS-COV has the amino acid sequence shown in SEQ ID NO:9. SARS-COV spike protein comprises S1 (SEQ ID NO:11) and S2 subunits. The S1 subunit comprises a receptor-binding domain (RBD) comprising; SEQ ID NO:12 or SEQ ID NO: 17, through which the SARSr-COV binds to ACE2 expressed by the host cells.

In various embodiments, the spike protein of SARS-COV-2 has the amino acid sequence shown in SEQ ID NO:10. SARS-COV-2 spike protein comprises S1 (SEQ ID NO:13 or SEQ ID NO: 16) and S2 subunits. The S1 subunit comprises a receptor-binding domain (RBD) comprising of SEQ ID NO:14 or SEQ ID NO: 15 through which the SARSr-CoV-2 binds to ACE2 expressed by host cells.

In various embodiments, the RBD of sarbecovirus spike protein refers to a polypeptide having the amino acid sequence shown in any one of SEQ ID NOS: 12, 14, 15, 17, or 26-30, or polypeptide having at least 75%, including one of 75%, 80%, 85%, 90%, 91%, 92%, 93%, 94%, 95%, 96%, 97%, 98%, 99% or greater amino acid sequence identity to any one of SEQ ID NOS: 12, 14, 15, 17, or 26-30. Such polypeptides may include e.g., isoforms, fragments, variants of the RBD of the spike protein encoded by SARS-COV-2, and the corresponding region of spike protein homologues from other SARSr-COV or other known sarbecoviruses.

In various embodiments, fragments of the RBD of sarbecovirus spike protein may have a minimum length of one of 10, 20, 30, 40, 50, 100, 150, 200 amino acids, and may have a maximum length of one of 20, 10, 20, 30, 40, 50, 100, 150, 200 amino acids.

In various embodiments, isoforms, fragments, variants or homologues may optionally be functional isoforms, fragments, variants or homologues, e.g., having a functional property/activity of the reference protein, as determined by analysis by a suitable assay for the functional property/activity where it binds and or enters a host cell via ACE2. For example, an isoform, fragment, variant or homologue of the spike protein of sarbecovirus may display association with ACE2.

In various embodiments, the sarbecovirus spike protein comprises a spike protein comprising or consisting of, an amino acid sequence having at least 75%, including one of 75%, 80%, 85%, 90%, 91%, 92%, 93%, 94%, 95%, 96%, 97%, 98%, 99% or 100% amino acid sequence identity to any one of consensus sarbecovirus spike protein SEQ ID NOS: 18-25 (SEQ ID NO:18; SEQ ID NO:19; SEQ ID NO: 20; SEQ ID NO: 21; SEQ ID NO: 22; SEQ ID NO: 23; SEQ ID NO: 24; or SEQ ID NO: 25). In various embodiments, the sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to any one of SEQ ID NOS: 9, 10, or 18-25.

In various embodiments, a fragment of sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NOS: 13-16. In various embodiments, a fragment of sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NOS:12, 14, 15, 17, or 26-30. In various embodiments, a fragment of sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NO:26. In various embodiments, a fragment of sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NO:27. In various embodiments, a fragment of sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NO:28. In various embodiments, a fragment of sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NO: 29. In various embodiments, a fragment of sarbecovirus spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NO: 30.

In some embodiments, a fragment of the RBD of SARS-COV-2 spike protein comprises, or consists of, an amino acid sequence having at least 75%, including one of 75%, 80%, 85%, 90%, 91%, 92%, 93%, 94%, 95%, 96%, 97%, 98%, 99% or 100% amino acid sequence identity to SEQ ID NOS:12, 14, 15, 17, or 26-30.

In various embodiments, the antigen-binding molecule comprises two antigen-binding molecules which binds to a sarbecovirus spike protein.

In various embodiments, the antigen-binding molecule binds to the receptor binding domain (RBD) of the Sarbecovirus spike protein.

In various embodiments, the antigen-binding molecule inhibits interaction between the sarbecovirus spike protein and Angiotensinogen converting enzyme 2 (ACE2).

In various embodiments, the antigen-binding molecule inhibits infection of ACE2-expressing cells by the sarbecovirus.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-CoV GD-1, SC2r-CoV RaTG13, SC2r-COV GX-P5L, SC1r-COV Rs2018B, SC1r-COV RsSHC014, SC1r-CoV LYRa11, SC1r-COV WIV-1, and SARS-COV.

In various embodiments, the sarbecovirus SARS-COV refers to the SARSr-COV having the nucleotide sequence of GenBank: NC_004718.3, reported in He et al., Biochem. Biophys. Res. Commun. 316 (2), 476-483 (2004), and encompasses variants thereof having a nucleotide sequence with at least 85% sequence identity (e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: NC_004718.3.

In various embodiments, the sarbecovirus SARS-COV-2 refers to the SARSr-CoV having the nucleotide sequence of GenBank: NC_045512.2, reported in Wu et al., Nature 579 (7798), 265-269 (2020), and encompasses variants thereof having a nucleotide sequence with at least 85% sequence identity (e.g. one of at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: NC_045512.2. In various embodiments, the sarbecovirus SARS-COV-2 variants comprise UK COVID-19 variant SARS-COV-2 B.1.1.7, the South African COVID-19 variant SARS-COV-2 B.1.351 also known as 20H/501Y.V2, or 501Y.V2 variant, Indian variant B1.617, and the Brazil variant P.1.

The antigen-binding molecules of the present disclosure may be designed and prepared using the sequences of monoclonal antibodies (mAbs) capable of binding to sarbecovirus spike protein. Antigen-binding regions of antibodies, such as single chain variable fragment (scFv), Fab and F(ab′)2 fragments may also be used/provided. An “antigen-binding region” is any fragment of an antibody which is capable of binding to the target for which the given antibody is specific. A mAbs is one of the most efficient and powerful tools for rapid development and deployment in fighting future emerging zoonotic viruses, and sarbecoviruses in particular.

Antibodies generally comprise six complementarity-determining regions (CDRs); three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody which binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term. In various embodiments, the VH region comprises the amino acid encoded by the nucleic acid sequence set forth in SEQ ID NO.31 and VL region comprises the amino acid encoded by the nucleic acid sequence set forth in SEQ ID NO. 32. In various embodiments, the VH region comprises the amino acid encoded by the nucleic acid sequence set forth in SEQ ID NO.33 and VL region comprises the amino acid encoded by the nucleic acid sequence set forth in SEQ ID NO. 34. In various embodiments, the VH region comprises the amino acid encoded by the nucleic acid sequence set forth in SEQ ID NO.35 and VL region comprises the amino acid encoded by the nucleic acid sequence set forth in SEQ ID NO. 36. In various embodiments, the VH region comprises the amino acid encoded by the nucleic acid sequence set forth in SEQ ID NO.37 and VL region comprises the amino acid encoded by the nucleic acid sequence set forth in SEQ ID NO. 38. In various embodiments, the VH region comprises the amino acid encoded by the nucleic acid sequence set forth in SEQ ID NO.39 and VL region comprises the amino acid encoded by the nucleic acid sequence set forth in SEQ ID NO. 40. In various embodiments, the VH region comprises the amino acid encoded by the nucleic acid sequence set forth in SEQ ID NO.41 and VL region comprises the amino acid encoded by the nucleic acid sequence set forth in SEQ ID NO. 42. In various embodiments, the VH region comprises the amino acid encoded by the nucleic acid sequence set forth in SEQ ID NO.43 and VL region comprises the amino acid encoded by the nucleic acid sequence set forth in SEQ ID NO. 44. In various embodiments, the VH region comprises the amino acid encoded by the nucleic acid sequence set forth in SEQ ID NO.45 and VL region comprises the amino acid encoded by the nucleic acid sequence set forth in SEQ ID NO. 46. In various embodiments, the VH region comprises the amino acid encoded by the nucleic acid sequence set forth in SEQ ID NO. 47 and VL region comprises the amino acid encoded by the nucleic acid sequence set forth in SEQ ID NO. 48. In various embodiments, the VH region comprises the amino acid encoded by the nucleic acid sequence set forth in SEQ ID NO.49 and VL region comprises the amino acid encoded by the nucleic acid sequence set forth in SEQ ID NO. 50. In various embodiments, the VH region comprises the amino acid encoded by the nucleic acid sequence set forth in SEQ ID NO.51 and VL region comprises the amino acid encoded by the nucleic acid sequence set forth in SEQ ID NO. 52. In various embodiments, the VH region comprises the amino acid encoded by the nucleic acid sequence set forth in SEQ ID NO. 53 and VL region comprises the amino acid encoded by the nucleic acid sequence set forth in SEQ ID NO. 54. In various embodiments, the VH region comprises the amino acid encoded by the nucleic acid sequence set forth in SEQ ID NO.55 and VL region comprises the amino acid encoded by the nucleic acid sequence set forth in SEQ ID NO. 56. In various embodiments, the VH region comprises the amino acid encoded by the nucleic acid sequence set forth in SEQ ID NO.57 and VL region comprises the amino acid encoded by the nucleic acid sequence set forth in SEQ ID NO. 58. In various embodiments, the VH region comprises the amino acid encoded by the nucleic acid sequence set forth in SEQ ID NO.59 and VL region comprises the amino acid encoded by the nucleic acid sequence set forth in SEQ ID NO. 60. In various embodiments, the VH region comprises the amino acid encoded by the nucleic acid sequence set forth in SEQ ID NO.61 and VL region comprises the amino acid encoded by the nucleic acid sequence set forth in SEQ ID NO. 62. In various embodiments, the VH region comprises the amino acid encoded by the nucleic acid sequence set forth in SEQ ID NO.63 and VL region comprises the amino acid encoded by the nucleic acid sequence set forth in SEQ ID NO. 64. In various embodiments, the VH region comprises the amino acid encoded by the nucleic acid sequence set forth in SEQ ID NO.65 and VL region comprises the amino acid encoded by the nucleic acid sequence set forth in SEQ ID NO. 66. In various embodiments, the VH region comprises the amino acid encoded by the nucleic acid sequence set forth in SEQ ID NO.67 and VL region comprises the amino acid encoded by the nucleic acid sequence set forth in SEQ ID NO. 68. In various embodiments, the VH region comprises the amino acid encoded by the nucleic acid sequence set forth in SEQ ID NO.69 and VL region comprises the amino acid encoded by the nucleic acid sequence set forth in SEQ ID NO. 70.

In various embodiments, the antigen-binding molecule comprises the CDRs of an antibody capable of binding to sarbecovirus spike protein described herein or comprises CDRs which are derived from an antibody capable of binding to sarbecovirus spike protein described herein. In some embodiments, the antigen-binding molecule comprises the FRs of an antibody capable of binding to sarbecovirus spike protein described herein or comprises FRs which are derived from an antibody capable of binding to sarbecovirus spike protein described herein. In some embodiments, the antigen-binding molecule comprises the CDRs and the FRs of an antibody capable of binding to sarbecovirus spike protein described herein or comprises CDRs and FRs which are derived from an antibody capable of binding to sarbecovirus spike protein described herein. That is, in some embodiments the antigen-binding molecule comprises the VH region and the VL region of an antibody capable of binding to sarbecovirus spike protein described herein or comprises VH and VL regions which are derived from an antibody capable of binding to sarbecovirus spike protein described herein.

In some embodiments the antigen-binding molecule comprises the CDRs, FRs and/or the VH and/or VL regions of an antibody capable of binding to sarbecovirus spike protein selected from any one of sarbecovirus spike protein of SARS-COV, SC2r-COV WIV-1, SC1r-COV RsSHC014, SARS-COV-2, SARS-COV-2 Alpha, SARS-COV-2 Beta, SARS-CoV-2 Delta, SC2r-COV RaTG13, SC2r-COV GD-1 and SC2r-COV GX-P5L.

In various embodiments, the antigen-binding molecule comprises:

(i) a heavy chain variable (VH) region incorporating the following CDRs:
HC-CDR1 having the amino acid sequence of SEQ ID NO:1
HC-CDR2 having the amino acid sequence of SEQ ID NO:2
HC-CDR3 having the amino acid sequence of SEQ ID NO:3; and

(ii) a light chain variable (VL) region incorporating the following CDRs:
LC-CDR1 having the amino acid sequence of SEQ ID NO: 4
LC-CDR2 having the amino acid sequence of SEQ ID NO: 5
LC-CDR3 having the amino acid sequence of SEQ ID NO: 6.

In various embodiments, the antigen-binding molecule comprises: a VH region comprising an amino acid sequence having at least 95% sequence identity to the amino acid sequence of SEQ ID NO: 2, 3 or 4; and a VL region comprising an amino acid sequence having at least 95% sequence identity to the amino acid sequence of SEQ ID NO: 5, 6 or 7.

In various embodiments the antigen-binding molecule comprises a VH region comprising an amino acid sequence having at least 75% sequence identity, including one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:1.

In various embodiments the antigen-binding molecule comprises a VH region comprising an amino acid sequence having at least 75% sequence identity, including one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 2.

In various embodiments the antigen-binding molecule comprises a VH region comprising an amino acid sequence having at least 75% sequence identity, including one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:3.

In various embodiments the antigen-binding molecule comprises a VL region comprising an amino acid sequence having at least 75% sequence identity, including one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 4.

In various embodiments the antigen-binding molecule comprises a VL region comprising an amino acid sequence having at least 75% sequence identity, including one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:5.

In various embodiments the antigen-binding molecule comprises a VL region comprising an amino acid sequence having at least 75% sequence identity, including one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:6.

In embodiments in accordance with the present disclosure in which one or more amino acids are substituted with another amino acid, the substitutions may be conservative substitutions, for example an aliphatic amino acid is replaced with another aliphatic amino acid such as a non-polar amino acid G, or A, or P, or I, or L, or V is replaced with a different non-polar amino acid; such as a polar uncharged amino acid C, or S, or T, or M, or N, or Q is replaced with a different polar uncharged amino acid such as a polar charged amino acid D, or E, or K, or R is replaced with a different polar charged amino acid or an aromatic amino acid is replaced with another aromatic amino acid such as H, or F, or W, or Y, is replaced with a different aromatic amino acid.

In variable embodiments, substitution(s) may be functionally conservative. That is, in some embodiments the substitution may not affect (or may not substantially affect) one or more functional properties (e.g., target binding) of the antigen-binding molecule comprising the substitution as compared to the equivalent unsubstituted molecule.

The VH and VL region of an antigen-binding region of an antibody together constitute the Fv region. In some embodiments, the antigen-binding molecule according to the present disclosure comprises, or consists of, an Fv region which binds to sarbecovirus spike protein. In various embodiments, the VH and VL regions of the Fv may be provided as single polypeptide joined by a linker region, i.e., a single chain Fv (scFv).

The VL and light chain constant (CL) region, and the VH region and heavy chain constant 1 (CH1) region of an antigen-binding region of an antibody together constitute the Fab region. In some embodiments the antigen-binding molecule comprises a Fab region comprising a VH, a CH1, a VL and a CL (e.g., Cκ or Cλ). In various embodiments, the Fab region comprises a polypeptide comprising a VH and a CH1 (e.g., a VH-CH1 fusion polypeptide), and a polypeptide comprising a VL and a CL (e.g., a VL-CL fusion polypeptide). In various embodiments, the Fab region comprises a polypeptide comprising a VH and a CL (e.g., a VH-CL fusion polypeptide) and a polypeptide comprising a VL and a CH (e.g., a VL-CH1 fusion polypeptide); that is, in some embodiments the Fab region is a CrossFab region. In various embodiments, the VH, CH1, VL and CL regions of the Fab or CrossFab are provided as single polypeptide joined by linker regions, i.e., as a single chain Fab (scFab) or a single chain CrossFab (scCrossFab).

In various embodiments, the antigen-binding molecule of the present disclosure comprises, or consists of, a Fab region which binds to sarbecovirus spike protein.

In various embodiments, the antigen-binding molecule described herein comprises, or consists of, a whole antibody which binds to sarbecovirus spike protein. As used herein, “whole antibody” refers to an antibody having a structure which is substantially similar to the structure of an immunoglobulin (Ig).

Immunoglobulins of type G (i.e., IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprise a VL followed by a CL. Depending on the heavy chain, immunoglobulins may be classed as IgG (e.g., IgG1, IgG2, IgG3, IgG4), IgA (e.g., IgA1, IgA2), IgD, IgE, or IgM. The light chain may be kappa (κ) or lambda (λ).

In some embodiments, the antigen-binding molecule described herein comprises, or consists of, an IgG (e.g., IgG1, IgG2, IgG3, IgG4), IgA (e.g. IgA1, IgA2), IgD, IgE, or IgM which binds to sarbecovirus spike protein.

In some embodiments the antigen-binding molecule of the present disclosure comprises one or more regions (e.g., CH1, CH2, CH3, etc.) of an immunoglobulin heavy chain constant sequence. In some embodiments the immunoglobulin heavy chain constant sequence is, or is derived from, the heavy chain constant sequence of an IgG (e.g., IgG1, IgG2, IgG3, IgG4), IgA (e.g., IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some the immunoglobulin heavy chain constant sequence is, or is derived from, the heavy chain constant sequence of a human IgG1 allotype (e.g., G1m1, G1m2, G1m3 or G1m17).

According to various embodiments there is an antigen-binding molecule as discussed herein above, for use in treatment or prevention of a disease caused by infection with a sarbecovirus.

In various embodiments, the antigen-binding molecule as discussed herein above, is suitable for use in individuals that have had SARS-COV-2 infection or vaccination.

In various embodiments, the antigen-binding molecule as discussed herein above, is suitable for use in individuals that are infection or vaccination naive to any sarbecovirus including SARS-COV, SARS-COV-2, SARS-COV-2 Alpha, or SARS-COV-2 Beta, SARS-COV-2 Delta.

In various embodiments, the antigen-binding molecule as discussed herein above, is suitable for use in treatment of individuals that have been diagnosed with a sarbecovirus infection.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-COV Rs2018B, SC1r-COV RsSHC014, SC1r-COV LYRa11, SC1r-COV WIV-1, and SARS-COV.

According to various embodiments there is use of an antigen-binding molecule as discussed herein above, in the manufacture of a medicament for use in treatment or prevention of a disease caused by infection with a sarbecovirus.

In various embodiments, the use of the antigen-binding molecule as discussed herein above in the manufacture of a medicament is suitable for use in treatment or prevention in individuals that have had SARS-COV-2 vaccination or infection.

In various embodiments, the use of an antigen-binding molecule as discussed herein above, is suitable for use in treatment or prevention in individuals that are infection or vaccination naive to any sarbecovirus including SARS-COV, SARS-COV-2, SARS-COV-2 Alphas, SARS-COV-2 Beta or SARs-COV-2 Delta.

In various embodiments, the use of the antigen-binding molecule as discussed herein above in the manufacture of a medicament is suitable for treatment in individuals that have been diagnosed with a sarbecovirus infection.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-CoV Rs2018B, SC1r-COV RsSHC014, SC1r-COV LYRa11, SC1r-COV WIV-1, and SARS-COV.

According to various embodiments there is a method of treating or preventing a disease caused by infection with a sarbecovirus, comprising administering to a subject a therapeutically or prophylactically effective amount of an antigen-binding molecule as discussed herein above.

In various embodiments, the subject is an individual that has had SARS-COV-2 vaccination or infection.

In various embodiments, the subject is an individual that has been diagnosed with a sarbecovirus infection

In various embodiments, the subject is an individual that are infection or vaccination naive to any sarbecovirus including SARS-COV, SARS-COV-2, SARS-COV-2 Alpha, SARS-COV-2 Beta or SARS-COV-2 Delta.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-COV Rs2018B, SC1r-COV RsSHC014, SC1r-COV LYRa11, SC1r-COV WIV-1, and SARS-COV.

According to various embodiments there is a nucleic acid, or a plurality of nucleic acids, optionally isolated, encoding an antigen-binding molecule as discussed herein above.

According to various embodiments there is an expression vector, or a plurality of expression vectors, comprising a nucleic acid or a plurality of nucleic acids as discussed herein above.

According to various embodiments there is a nucleic acid or a plurality of nucleic acids as discussed herein above, or an expression vector, or a plurality of expression vectors comprising the nucleic acid or the plurality of nucleic acids, capable of expressing an antigen-binding molecule as discussed herein above, for use in treatment or prevention of a disease caused by infection with a sarbecovirus.

In various embodiments, the nucleic acid or the plurality of nucleic acids as discussed herein above, or the expression vector, or the plurality of expression vectors comprising the nucleic acid or the plurality of nucleic acids, capable of expressing an antigen-binding molecule as discussed herein above, is suitable for use in individuals that have had SARS-COV-2 infection or vaccination.

In various embodiments, the nucleic acid or the plurality of nucleic acids as discussed herein above, or the expression vector, or the plurality of expression vectors comprising the nucleic acid or the plurality of nucleic acids, capable of expressing an antigen-binding molecule as discussed herein above, is suitable for use in individuals that are infection or vaccination naive to any sarbecovirus including SARS-COV, SARS-COV-2, SARS-COV-2 B.1.1.7, or SARS-COV-2 B.1.351.

In various embodiments, the nucleic acid or the plurality of nucleic acids as discussed herein above, or the expression vector, or the plurality of expression vectors comprising the nucleic acid or the plurality of nucleic acids, capable of expressing an antigen-binding molecule as discussed herein above, is suitable for use in treatment of individuals that have been diagnosed with a sarbecovirus infection.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-COV Rs2018B, SC1r-COV RsSHC014, SC1r-COV LYRa11, SC1r-COV WIV-1, and SARS-COV.

According to various embodiments there is a nucleic acid or a plurality of nucleic acids as discussed herein above, or an expression vector or a plurality of expression vectors comprising the nucleic acid or the plurality of nucleic acids, capable of expressing an antigen-binding molecule as discussed herein above, in the manufacture of a medicament for use in treatment or prevention of a disease caused by infection with a sarbecovirus.

In various embodiments, use of the nucleic acid or the plurality of nucleic acids as discussed herein above, the expression vector or the plurality of expression vectors comprising the nucleic acid or the plurality of nucleic acids, capable of expressing an antigen-binding molecule as discussed herein above, in the manufacture of a medicament is suitable for use in treatment or prevention in individuals that have had SARS-COV-2 vaccination or infection.

In various embodiments, use of the nucleic acid or the plurality of nucleic acids as discussed herein above, the expression vector or the plurality of expression vectors comprising the nucleic acid or the plurality of nucleic acids, capable of expressing an antigen-binding molecule as discussed herein above, in the manufacture of a medicament is suitable for use in treatment or prevention in individuals that are infection or vaccination naive to any sarbecovirus including SARS-COV, SARS-COV-2, SARS-COV-2 Alpha, SARS-COV-2 Beta or SARS-COV-2 Delta.

In various embodiments, use of the nucleic acid or the plurality of nucleic acids as discussed herein above, the expression vector or the plurality of expression vectors comprising the nucleic acid or the plurality of nucleic acids, capable of expressing an antigen-binding molecule as discussed herein above, in the manufacture of a medicament is suitable for use in treatment in individuals have been diagnosed with a sarbecovirus infection.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-COV Rs2018B, SC1r-COV RsSHC014, SC1r-COV LYRa11, SC1r-COV WIV-1, SARS-COV.

According to various embodiments there is a method of treating or preventing a disease caused by infection with a sarbecovirus, comprising administering to a subject a therapeutically or prophylactically effective amount of an antigen-binding molecule wherein the antigen-binding molecule is expressed by a nucleic acid or a plurality of nucleic acids as discussed herein above, or expressed in an expression vector or a plurality of expression vectors as discussed herein above.

In various embodiments, the subject is an individual that has had SARS-COV-2 vaccination or infection.

In various embodiments, the subject is an individual that is infection or vaccination naive to any sarbecovirus including SARS-COV, SARS-COV-2, SARS-COV-2 Alpha, SARS-COV-2 Beta or SARS-COV-2 Delta.

In various embodiments, the subject is an individual that has been diagnosed with a sarbecovirus infection.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-COV Rs2018B, SC1r-COV RsSHC014, SC1r-COV LYRa11, SC1r-COV WIV-1, and SARS-COV.

According to various embodiments there is a cell comprising an antigen-binding molecule as discussed herein above, a nucleic acid or a plurality of nucleic acids as discussed herein above, or an expression vector or a plurality of expression vectors as discussed herein above.

According to various embodiments there is a method for producing an antigen-binding molecule which binds to a sarbecovirus spike protein, comprising culturing a cell as discussed herein above under conditions suitable for expression of an antigen-binding molecule by the cell.

According to various embodiments there is a cell as discussed herein above, for use in treatment or prevention of a disease caused by infection with a sarbecovirus.

In various embodiments, the cell as discussed herein above, is suitable for use in individuals that have had SARS-COV-2 infection or vaccination.

In various embodiments, the cell as discussed herein above, is suitable for use in individuals that are infection or vaccination naive to any sarbecovirus including SARS-CoV, SARS-COV-2, SARS-COV-2 Alpha, SARS-COV-2 Beta or SARS-COV-2 Delta.

In various embodiments, the cell as discussed herein above, is suitable for use in treatment of individuals that have been diagnosed with a sarbecovirus infection.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-CoV Rs2018B, SC1r-COV RsSHC014, SC1r-COV LYRa11, SC1r-COV WIV-1, and SARS-COV.

According to various embodiments there is use of a cell as discussed herein above, in the manufacture of a medicament for use in treatment or prevention of a disease caused by infection with a sarbecovirus.

In various embodiments, use of the cell as discussed herein above, in the manufacture of a medicament is suitable for use in individuals that have had SARS-COV-2 vaccination or infection.

In various embodiments, use of the cell as discussed herein above, in the manufacture of a medicament is suitable for use in individuals that are infection or vaccination naive to any sarbecovirus including SARS-COV, SARS-COV-2, SARS-COV-2 Alpha, SARS-COV-2 Beta or SARS-COV-2 Delta.

In various embodiments, use of the cell as discussed herein above, in the manufacture of a medicament is suitable for use in treatment of individuals that have been diagnosed with a sarbecovirus infection.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-CoV Rs2018B, SC1r-COV RsSHC014, SC1r-COV LYRa11, SC1r-COV WIV-1, and SARS-COV.

According to various embodiments there is a method of treating or preventing a disease caused by infection with a sarbecovirus, comprising administering to a subject a therapeutically or prophylactically effective amount of an antigen-binding molecule as discussed herein above, wherein the antigen-binding molecule is expressed in a cell as discussed herein above.

In various embodiments, the subject is an individual that have had SARS-COV-2 vaccination.

In various embodiments, the subject is an individual that is infection or vaccination naive to any sarbecovirus including SARS-COV, SARS-COV-2, SARS-COV-2 Alpha, SARS-COV-2 Beta SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, or SARS-COV-2 Delta.

In various embodiments, the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-CoV RaTG13, SC2r-COV GX-P5L, SC1r-COV Rs2018B, SC1r-COV RsSHC014, SC1r-COV LYRa11, SC1r-COV WIV-1, and SARS-COV.

According to various embodiments there is a composition comprising an antigen-binding molecule as discussed herein above, a nucleic acid or a plurality of nucleic acids as discussed herein above, an expression vector or a plurality of expression vectors as discussed herein above, or a cell as discussed herein above, and a pharmaceutically acceptable carrier, diluent, excipient or adjuvant.

According to various embodiments there is a composition as discussed herein above, for use in treatment or prevention of a disease caused by infection with a sarbecovirus.

In various embodiments, the composition as discussed herein above, is suitable for use in individuals that have had SARS-COV-2 infection or vaccination.

In various embodiments, the composition as discussed herein above, is suitable for use in individuals that are infection or vaccination naive to any sarbecovirus including SARS-COV, SARS-COV-2, SARS-COV-2 Alpha, SARS-COV-2 Beta or SARS-COV-2 Delta.

In various embodiments, the composition as discussed herein above, is suitable for use in treatment of individuals that have been diagnosed with a sarbecovirus infection.

In various embodiments, the sarbecovirus is selected from the group comprising SARS-COV-2, SARS-COV-2 B.1.1.7, SARS-COV-2 B.1.351, SARS-COV-2 B.1.617.2, SARS-CoV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-COV RaTG13, SC2r-COV GX-P5L, SC1r-COV Rs2018B, SC1r-COV RsSHC014, SC1r-COV LYRa11, SC1r-CoV WIV-1, and SARS-COV.

According to various embodiments there is use of a composition as discussed herein above, in the manufacture of a medicament for use in treatment or prevention of a disease caused by infection with a sarbecovirus.

In various embodiments, use of the composition as discussed herein above, in the manufacture of a medicament is suitable for use in individuals that have had SARS-COV-2 vaccination.

In various embodiments, use of the composition as discussed herein above, in the manufacture of a medicament is suitable for use in individuals that are infection or vaccination naive to any sarbecovirus including SARS-COV, SARS-COV-2, SARS-COV-2 Alpha, SARS-COV-2 Beta or SARS-COV-2 Delta.

In various embodiments, use of the composition as discussed herein above, in the manufacture of a medicament is suitable for use in treatment of individuals that have been diagnosed with a sarbecovirus infection.

In various embodiments, the sarbecovirus is selected from the group comprising SARS-COV-2, SARS-COV-2 B.1.1.7, SARS-COV-2 B.1.351, SARS-COV-2 B.1.617.2, SARS-CoV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-COV RaTG13, SC2r-COV GX-P5L, SC1r-COV Rs2018B, SC1r-COV RsSHC014, SC1r-COV LYRa11, SC1r-CoV WIV-1, and SARS-COV.

According to various embodiments there is a method of treating or preventing a disease caused by infection with a sarbecovirus, comprising administering to a subject a therapeutically or prophylactically effective amount of a composition as discussed herein above.

In various embodiments, the subject is an individual that have had SARS-COV-2 vaccination or infection.

In various embodiments, the subject is an individual that are infection or vaccination naive to any sarbecovirus including SARS-COV, SARS-COV-2, SARS-COV-2 Alpha, SARS-COV-2 Beta or SARS-COV-2 Delta.

In various embodiments, the subject is an individual that has been diagnosed with a sarbecovirus infection.

In various embodiments, the sarbecovirus is selected from the group comprising SARS-COV, SC2r-COV WIV-1, SC1r-COV RsSHC014, SARS-COV-2, SARS-COV-2 Alpha, SARS-COV-2 Beta, SARS-COV-2 Delta, SC2r-COV RaTG13, SC2r-COV GD-1 and SC2r-CoV GX-P5L.

According to various embodiments there is use of an antigen-binding molecule as discussed herein above to inhibit infection of ACE2-expressing cells by a sarbecovirus.

In various embodiments, the sarbecovirus is selected from the group comprising SARS-COV, SC2r-COV WIV-1, SC1r-CoV RsSHC014, SARS-COV-2, SARS-COV-2 Alpha, SARS-COV-2 Beta, SARS-COV-2 Delta, SC2r-COV RaTG13, SC2r-COV GD-1 and SC2r-CoV GX-P5L

Sequences

| SEQ ID | | |
|---|---|---|
| NO: | DESCRIPTION | SEQUENCE |
| 1 | Heavy chain | GFTFSXYX |
| | variable region | |
| | HC-CDR1 | |
| 2 | Heavy chain | IYXXGXT |
| | variable region | |
| | HC-CDR2 | |
| 3 | Heavy chain | ARXXXXXFDY |
| | variable region | |
| | HC-CDR3 | |
| 4 | light chain | QSIXTY |
| | variable region | |
| | LC-CDR1 | |
| 5 | Light chain | XAS |
| | variable region | |
| | LC-CDR2 | |
| 6 | Light chain | QQSXXTXPXT |
| | variable region | |
| | LC-CDR3 | |
| 7 | SARS-CoV | ATATTAGGTTTTTACCTACCCAGGAAAAGCCAACCAACCTCGATCTCTTGTAGATCTG |
| | (GenBank: | TTCTCTAAACGAACTTTAAAATCTGTGTAGCTGTCGCTCGGCTGCATGCCTAGTGCAC |
| | NC_004718.3) | CTACGCAGTATAAACAATAATAAATTTTACTGTCGTTGACAAGAAACGAGTAACTCGT |
| | | CCCTCTTCTGCAGACTGCTTACGGTTTCGTCCGTGTTGCAGTCGATCATCAGCATAC |
| | | CTAGGTTTCGTCCGGGTGTGACCGAAAGGTAAGATGGAGAGCCTTGTTCTTGGTGTC |
| | | AACGAGAAAACACACGTCCAACTCAGTTTGCCTGTCCTTCAGGTTAGAGACGTGCTA |
| | | GTGCGTGGCTTCGGGGACTCTGTGGAAGAGGCCCTATCGGAGGCACGTGAACACCT |
| | | CAAAAATGGCACTTGTGGTCTAGTAGAGCTGGAAAAAGGCGTACTGCCCCAGCTTGA |
| | | ACAGCCCTATGTGTTCATTAAACGTTCTGATGCCTTAAGCACCAATCACGGCCACAA |
| | | GGTCGTTGAGCTGGTTGCAGAAATGGACGGCATTCAGTACGGTCGTAGCGGTATAA |
| | | CACTGGGAGTACTCGTGCCACATGTGGGCGAAACCCCAATTGCATACCGCAATGTTC |
| | | TTCTTCGTAAGAACGGTAATAAGGGAGCCGGTGGTCATAGCTATGGCATCGATCTAA |
| | | AGTCTTATGACTTAGGTGACGAGCTTGGCACTGATCCCATTGAAGATTATGAACAAAA |
| | | CTGGAACACTAAGCATGGCAGTGGTGCACTCCGTGAACTCACTCGTGAGCTCAATG |
| | | GAGGTGCAGTCACTCGCTATGTCGACAACAATTTCTGTGGCCCAGATGGGTACCCTC |
| | | TTGATTGCATCAAAGATTTTCTCGCACGCGCGGGCAAGTCAATGTGCACTCTTTCCG |
| | | AACAACTTGATTACATCGAGTCGAAGAGAGGTGTCTACTGCTGCCGTGACCATGAGC |
| | | ATGAAATTGCCTGGTTCACTGAGCGCTCTGATAAGAGCTACGAGCACCAGACACCCT |
| | | TCGAAATTAAGAGTGCCAAGAAATTTGACACTTTCAAAGGGGAATGCCCAAAGTTTGT |
| | | GTTTCCTCTTAACTCAAAAGTCAAAGTCATTCAACCACGTGTTGAAAAGAAAAAGACT |
| | | GAGGGTTTCATGGGGCGTATACGCTCTGTGTACCCTGTTGCATCTCCACAGGAGTGT |
| | | AACAATATGCACTTGTCTACCTTGATGAAATGTAATCATTGCGATGAAGTTTCATGGC |
| | | AGACGTGCGACTTTCTGAAAGCCACTTGTGAACATTGTGGCACTGAAAATTTAGTTAT |
| | | TGAAGGACCTACTACATGTGGGTACCTACCTACTAATGCTGTAGTGAAAATGCCATGT |
| | | CCTGCCTGTCAAGACCCAGAGATTGGACCTGAGCATAGTGTTGCAGATTATCACAAC |
| | | CACTCAAACATTGAAACTCGACTCCGCAAGGGAGGTAGGACTAGATGTTTTGGAGGC |
| | | TGTGTGTTTGCCTATGTTGGCTGCTATAATAAGCGTGCCTACTGGGTTCCTCGTGCTA |
| | | GTGCTGATATTGGCTCAGGCCATACTGGCATTACTGGTGACAATGTGGAGACCTTGA |
| | | ATGAGGATCTCCTTGAGATACTGAGTCGTGAACGTGTTAACATTAACATTGTTGGCGA |
| | | TTTTCATTTGAATGAAGAGGTTGCCATCATTTTGGCATCTTTCTCTGCTTCTACAAGTG |
| | | CCTTTATTGACACTATAAAGAGTCTTGATTACAAGTCTTTCAAAACCATTGTTGAGTCC |
| | | TGCGGTAACTATAAAGTTACCAAGGGAAAGCCCGTAAAAGGTGCTTGGAACATTGGA |
| | | CAACAGAGATCAGTTTTAACACCACTGTGTGGTTTTCCCTCACAGGCTGCTGGTGTTA |
| | | TCAGATCAATTTTTGCGCGCACACTTGATGCAGCAAACCACTCAATTCCTGATTTGCA |
| | | AAGAGCAGCTGTCACCATACTTGATGGTATTTCTGAACAGTCATTACGTCTTGTCGAC |
| | | GCCATGGTTTATACTTCAGACCTGCTCACCAACAGTGTCATTATTATGGCATATGTAA |
| | | CTGGTGGTCTTGTACAACAGACTTCTCAGTGGTTGTCTAATCTTTTGGGCACTACTGT |
| | | TGAAAAACTCAGGCCTATCTTTGAATGGATTGAGGCGAAACTTAGTGCAGGAGTTGA |
| | | ATTTCTCAAGGATGCTTGGGAGATTCTCAAATTTCTCATTACAGGTGTTTTTGACATC |
| | | GTCAAGGGTCAAATACAGGTTGCTTCAGATAACATCAAGGATTGTGTAAAATGCTTCA |
| | | TTGATGTTGTTAACAAGGCACTCGAAATGTGCATTGATCAAGTCACTATCGCTGGCG |
| | | CAAAGTTGCGATCACTCAACTTAGGTGAAGTCTTCATCGCTCAAAGCAAGGGACTTTA |
| | | CCGTCAGTGTATACGTGGCAAGGAGCAGCTGCAACTACTCATGCCTCTTAAGGCACC |
| | | AAAAGAAGTAACCTTTCTTGAAGGTGATTCACATGACACAGTACTTACCTCTGAGGAG |
| | | GTTGTTCTCAAGAACGGTGAACTCGAAGCACTCGAGACGCCCGTTGATAGCTTCACA |
| | | AATGGAGCTATCGTTGGCACACCAGTCTGTGTAAATGGCCTCATGCTCTTAGAGATT |
| | | AAGGACAAAGAACAATACTGCGCATTGTCTCCTGGTTTACTGGCTACAAACAATGTCT |
| | | TTCGCTTAAAAGGGGGTGCACCAATTAAAGGTGTAACCTTTGGAGAAGATACTGTTT |
| | | GGGAAGTTCAAGGTTACAAGAATGTGAGAATCACATTTGAGCTTGATGAACGTGTTG |
| | | ACAAAGTGCTTAATGAAAAGTGCTCTGTCTACACTGTTGAATCCGGTACCGAAGTTAC |
| | | TGAGTTTGCATGTGTTGTAGCAGAGGCTGTTGTGAAGACTTTACAACCAGTTTCTGAT |
| | | CTCCTTACCAACATGGGTATTGATCTTGATGAGTGGAGTGTAGCTACATTCTACTTAT |
| | | TTGATGATGCTGGTGAAGAAAACTTTTCATCACGTATGTATTGTTCCTTTTACCCTCCA |
| | | GATGAGGAAGAAGAGGACGATGCAGAGTGTGAGGAAGAAGAAATTGATGAAACCTG |
| | | TGAACATGAGTACGGTACAGAGGATGATTATCAAGGTCTCCCTCTGGAATTTGGTGC |
| | | CTCAGCTGAAACAGTTCGAGTTGAGGAAGAAGAAGAGGAAGACTGGCTGGATGATA |
| | | CTACTGAGCAATCAGAGATTGAGCCAGAACCAGAACCTACACCTGAAGAACCAGTTA |
| | | ATCAGTTTACTGGTTATTTAAAACTTACTGACAATGTTGCCATTAAATGTGTTGACATC |
| | | GTTAAGGAGGCACAAAGTGCTAATCCTATGGTGATTGTAAATGCTGCTAACATACACC |
| | | TGAAACATGGTGGTGGTGTAGCAGGTGCACTCAACAAGGCAACCAATGGTGCCATG |
| | | CAAAAGGAGAGTGATGATTACATTAAGCTAAATGGCCCTCTTACAGTAGGAGGGTCT |
| | | TGTTTGCTTTCTGGACATAATCTTGCTAAGAAGTGTCTGCATGTTGTTGGACCTAACC |
| | | TAAATGCAGGTGAGGACATCCAGCTTCTTAAGGCAGCATATGAAAATTTCAATTCACA |
| | | GGACATCTTACTTGCACCATTGTTGTCAGCAGGCATATTTGGTGCTAAACCACTTCAG |
| | | TCTTTACAAGTGTGCGTGCAGACGGTTCGTACACAGGTTTATATTGCAGTCAATGACA |
| | | AAGCTCTTTATGAGCAGGTTGTCATGGATTATCTTGATAACCTGAAGCCTAGAGTGGA |
| | | AGCACCTAAACAAGAGGAGCCACCAAACACAGAAGATTCCAAAACTGAGGAGAAATC |
| | | TGTCGTACAGAAGCCTGTCGATGTGAAGCCAAAAATTAAGGCCTGCATTGATGAGGT |
| | | TACCACAACACTGGAAGAAACTAAGTTTCTTACCAATAAGTTACTCTTGTTTGCTGATA |
| | | TCAATGGTAAGCTTTACCATGATTCTCAGAACATGCTTAGAGGTGAAGATATGTCTTT |
| | | CCTTGAGAAGGATGCACCTTACATGGTAGGTGATGTTATCACTAGTGGTGATATCACT |
| | | TGTGTTGTAATACCCTCCAAAAAGGCTGGTGGCACTACTGAGATGCTCTCAAGAGCT |
| | | TTGAAGAAAGTGCCAGTTGATGAGTATATAACCACGTACCCTGGACAAGGATGTGCT |
| | | GGTTATACACTTGAGGAAGCTAAGACTGCTCTTAAGAAATGCAAATCTGCATTTTATG |
| | | TACTACCTTCAGAAGCACCTAATGCTAAGGAAGAGATTCTAGGAACTGTATCCTGGAA |
| | | TTTGAGAGAAATGCTTGCTCATGCTGAAGAGACAAGAAAATTAATGCCTATATGCATG |
| | | GATGTTAGAGCCATAATGGCAACCATCCAACGTAAGTATAAAGGAATTAAAATTCAAG |
| | | AGGGCATCGTTGACTATGGTGTCCGATTCTTCTTTTATACTAGTAAAGAGCCTGTAGC |
| | | TTCTATTATTACGAAGCTGAACTCTCTAAATGAGCCGCTTGTCACAATGCCAATTGGT |
| | | TATGTGACACATGGTTTTAATCTTGAAGAGGCTGCGCGCTGTATGCGTTCTCTTAAAG |
| | | CTCCTGCCGTAGTGTCAGTATCATCACCAGATGCTGTTACTACATATAATGGATACCT |
| | | CACTTCGTCATCAAAGACATCTGAGGAGCACTTTGTAGAAACAGTTTCTTTGGCTGGC |
| | | TCTTACAGAGATTGGTCCTATTCAGGACAGCGTACAGAGTTAGGTGTTGAATTTCTTA |
| | | AGCGTGGTGACAAAATTGTGTACCACACTCTGGAGAGCCCCGTCGAGTTTCATCTTG |
| | | ACGGTGAGGTTCTTTCACTTGACAAACTAAAGAGTCTCTTATCCCTGCGGGAGGTTA |
| | | AGACTATAAAAGTGTTCACAACTGTGGACAACACTAATCTCCACACACAGCTTGTGGA |
| | | TATGTCTATGACATATGGACAGCAGTTTGGTCCAACATACTTGGATGGTGCTGATGTT |
| | | ACAAAAATTAAACCTCATGTAAATCATGAGGGTAAGACTTTCTTTGTACTACCTAGTGA |
| | | TGACACACTACGTAGTGAAGCTTTCGAGTACTACCATACTCTTGATGAGAGTTTTCTT |
| | | GGTAGGTACATGTCTGCTTTAAACCACACAAAGAAATGGAAATTTCCTCAAGTTGGTG |
| | | GTTTAACTTCAATTAAATGGGCTGATAACAATTGTTATTTGTCTAGTGTTTTATTAGCA |
| | | CTTCAACAGCTTGAAGTCAAATTCAATGCACCAGCACTTCAAGAGGCTTATTATAGAG |
| | | CCCGTGCTGGTGATGCTGCTAACTTTTGTGCACTCATACTCGCTTACAGTAATAAAAC |
| | | TGTTGGCGAGCTTGGTGATGTCAGAGAAACTATGACCCATCTTCTACAGCATGCTAA |
| | | TTTGGAATCTGCAAAGCGAGTTCTTAATGTGGTGTGTAAACATTGTGGTCAGAAAACT |
| | | ACTACCTTAACGGGTGTAGAAGCTGTGATGTATATGGGTACTCTATCTTATGATAATC |
| | | TTAAGACAGGTGTTTCCATTCCATGTGTGTGTGGTCGTGATGCTACACAATATCTAGT |
| | | ACAACAAGAGTCTTCTTTTGTTATGATGTCTGCACCACCTGCTGAGTATAAATTACAG |
| | | CAAGGTACATTCTTATGTGCGAATGAGTACACTGGTAACTATCAGTGTGGTCATTACA |
| | | CTCATATAACTGCTAAGGAGACCCTCTATCGTATTGACGGAGCTCACCTTACAAAGAT |
| | | GTCAGAGTACAAAGGACCAGTGACTGATGTTTTCTACAAGGAAACATCTTACACTACA |
| | | ACCATCAAGCCTGTGTCGTATAAACTCGATGGAGTTACTTACACAGAGATTGAACCAA |
| | | AATTGGATGGGTATTATAAAAAGGATAATGCTTACTATACAGAGCAGCCTATAGACCT |
| | | TGTACCAACTCAACCATTACCAAATGCGAGTTTTGATAATTTCAAACTCACATGTTCTA |
| | | ACACAAAATTTGCTGATGATTTAAATCAAATGACAGGCTTCACAAAGCCAGCTTCACG |
| | | AGAGCTATCTGTCACATTCTTCCCAGACTTGAATGGCGATGTAGTGGCTATTGACTAT |
| | | AGACACTATTCAGCGAGTTTCAAGAAAGGTGCTAAATTACTGCATAAGCCAATTGTTT |
| | | GGCACATTAACCAGGCTACAACCAAGACAACGTTCAAACCAAACACTTGGTGTTTAC |
| | | GTTGTCTTTGGAGTACAAAGCCAGTAGATACTTCAAATTCATTTGAAGTTCTGGCAGT |
| | | AGAAGACACACAAGGAATGGACAATCTTGCTTGTGAAAGTCAACAACCCACCTCTGA |
| | | AGAAGTAGTGGAAAATCCTACCATACAGAAGGAAGTCATAGAGTGTGACGTGAAAAC |
| | | TACCGAAGTTGTAGGCAATGTCATACTTAAACCATCAGATGAAGGTGTTAAAGTAACA |
| | | CAAGAGTTAGGTCATGAGGATCTTATGGCTGCTTATGTGGAAAACACAAGCATTACC |
| | | ATTAAGAAACCTAATGAGCTTTCACTAGCCTTAGGTTTAAAAACAATTGCCACTCATG |
| | | GTATTGCTGCAATTAATAGTGTTCCTTGGAGTAAAATTTTGGCTTATGTCAAACCATTC |
| | | TTAGGACAAGCAGCAATTACAACATCAAATTGCGCTAAGAGATTAGCACAACGTGTGT |
| | | TTAACAATTATATGCCTTATGTGTTTACATTATTGTTCCAATTGTGTACTTTTACTAAAA |
| | | GTACCAATTCTAGAATTAGAGCTTCACTACCTACAACTATTGCTAAAAATAGTGTTAAG |
| | | AGTGTTGCTAAATTATGTTTGGATGCCGGCATTAATTATGTGAAGTCACCCAAATTTT |
| | | CTAAATTGTTCACAATCGCTATGTGGCTATTGTTGTTAAGTATTTGCTTAGGTTCTCTA |
| | | ATCTGTGTAACTGCTGCTTTTGGTGTACTCTTATCTAATTTTGGTGCTCCTTCTTATTG |
| | | TAATGGCGTTAGAGAATTGTATCTTAATTCGTCTAACGTTACTACTATGGATTTCTGTG |
| | | AAGGTTCTTTTCCTTGCAGCATTTGTTTAAGTGGATTAGACTCCCTTGATTCTTATCCA |
| | | GCTCTTGAAACCATTCAGGTGACGATTTCATCGTACAAGCTAGACTTGACAATTTTAG |
| | | GTCTGGCCGCTGAGTGGGTTTTGGCATATATGTTGTTCACAAAATTCTTTTATTTATTA |
| | | GGTCTTTCAGCTATAATGCAGGTGTTCTTTGGCTATTTTGCTAGTCATTTCATCAGCA |
| | | ATTCTTGGCTCATGTGGTTTATCATTAGTATTGTACAAATGGCACCCGTTTCTGCAAT |
| | | GGTTAGGATGTACATCTTCTTTGCTTCTTTCTACTACATATGGAAGAGCTATGTTCATA |
| | | TCATGGATGGTTGCACCTCTTCGACTTGCATGATGTGCTATAAGCGCAATCGTGCCA |
| | | CACGCGTTGAGTGTACAACTATTGTTAATGGCATGAAGAGATCTTTCTATGTCTATGC |
| | | AAATGGAGGCCGTGGCTTCTGCAAGACTCACAATTGGAATTGTCTCAATTGTGACAC |
| | | ATTTTGCACTGGTAGTACATTCATTAGTGATGAAGTTGCTCGTGATTTGTCACTCCAG |
| | | TTTAAAAGACCAATCAACCCTACTGACCAGTCATCGTATATTGTTGATAGTGTTGCTG |
| | | TGAAAAATGGCGCGCTTCACCTCTACTTTGACAAGGCTGGTCAAAAGACCTATGAGA |
| | | GACATCCGCTCTCCCATTTTGTCAATTTAGACAATTTGAGAGCTAACAACACTAAAGG |
| | | TTCACTGCCTATTAATGTCATAGTTTTTGATGGCAAGTCCAAATGCGACGAGTCTGCT |
| | | TCTAAGTCTGCTTCTGTGTACTACAGTCAGCTGATGTGCCAACCTATTCTGTTGCTTG |
| | | ACCAAGCTCTTGTATCAGACGTTGGAGATAGTACTGAAGTTTCCGTTAAGATGTTTGA |
| | | TGCTTATGTCGACACCTTTTCAGCAACTTTTAGTGTTCCTATGGAAAAACTTAAGGCA |
| | | CTTGTTGCTACAGCTCACAGCGAGTTAGCAAAGGGTGTAGCTTTAGATGGTGTCCTT |
| | | TCTACATTCGTGTCAGCTGCCCGACAAGGTGTTGTTGATACCGATGTTGACACAAAG |
| | | GATGTTATTGAATGTCTCAAACTTTCACATCACTCTGACTTAGAAGTGACAGGTGACA |
| | | GTTGTAACAATTTCATGCTCACCTATAATAAGGTTGAAAACATGACGCCCAGAGATCT |
| | | TGGCGCATGTATTGACTGTAATGCAAGGCATATCAATGCCCAAGTAGCAAAAAGTCA |
| | | CAATGTTTCACTCATCTGGAATGTAAAAGACTACATGTCTTTATCTGAACAGCTGCGT |
| | | AAACAAATTCGTAGTGCTGCCAAGAAGAACAACATACCTTTTAGACTAACTTGTGCTA |
| | | CAACTAGACAGGTTGTCAATGTCATAACTACTAAAATCTCACTCAAGGGTGGTAAGAT |
| | | TGTTAGTACTTGTTTTAAACTTATGCTTAAGGCCACATTATTGTGCGTTCTTGCTGCAT |
| | | TGGTTTGTTATATCGTTATGCCAGTACATACATTGTCAATCCATGATGGTTACACAAAT |
| | | GAAATCATTGGTTACAAAGCCATTCAGGATGGTGTCACTCGTGACATCATTTCTACTG |
| | | ATGATTGTTTTGCAAATAAACATGCTGGTTTTGACGCATGGTTTAGCCAGCGTGGTGG |
| | | TTCATACAAAAATGACAAAAGCTGCCCTGTAGTAGCTGCTATCATTACAAGAGAGATT |
| | | GGTTTCATAGTGCCTGGCTTACCGGGTACTGTGCTGAGAGCAATCAATGGTGACTTC |
| | | TTGCATTTTCTACCTCGTGTTTTTAGTGCTGTTGGCAACATTTGCTACACACCTTCCAA |
| | | ACTCATTGAGTATAGTGATTTTGCTACCTCTGCTTGCGTTCTTGCTGCTGAGTGTACA |
| | | ATTTTTAAGGATGCTATGGGCAAACCTGTGCCATATTGTTATGACACTAATTTGCTAG |
| | | AGGGTTCTATTTCTTATAGTGAGCTTCGTCCAGACACTCGTTATGTGCTTATGGATGG |
| | | TTCCATCATACAGTTTCCTAACACTTACCTGGAGGGTTCTGTTAGAGTAGTAACAACT |
| | | TTTGATGCTGAGTACTGTAGACATGGTACATGCGAAAGGTCAGAAGTAGGTATTTGC |
| | | CTATCTACCAGTGGTAGATGGGTTCTTAATAATGAGCATTACAGAGCTCTATCAGGAG |
| | | TTTTCTGTGGTGTTGATGCGATGAATCTCATAGCTAACATCTTTACTCCTCTTGTGCAA |
| | | CCTGTGGGTGCTTTAGATGTGTCTGCTTCAGTAGTGGCTGGTGGTATTATTGCCATAT |
| | | TGGTGACTTGTGCTGCCTACTACTTTATGAAATTCAGACGTGTTTTTGGTGAGTACAA |
| | | CCATGTTGTTGCTGCTAATGCACTTTTGTTTTTGATGTCTTTCACTATACTCTGTCTGG |
| | | TACCAGCTTACAGCTTTCTGCCGGGAGTCTACTCAGTCTTTTACTTGTACTTGACATT |
| | | CTATTTCACCAATGATGTTTCATTCTTGGCTCACCTTCAATGGTTTGCCATGTTTTCTC |
| | | CTATTGTGCCTTTTTGGATAACAGCAATCTATGTATTCTGTATTTCTCTGAAGCACTGC |
| | | CATTGGTTCTTTAACAACTATCTTAGGAAAAGAGTCATGTTTAATGGAGTTACATTTAG |
| | | TACCTTCGAGGAGGCTGCTTTGTGTACCTTTTTGCTCAACAAGGAAATGTACCTAAAA |
| | | TTGCGTAGCGAGACACTGTTGCCACTTACACAGTATAACAGGTATCTTGCTCTATATA |
| | | ACAAGTACAAGTATTTCAGTGGAGCCTTAGATACTACCAGCTATCGTGAAGCAGCTT |
| | | GCTGCCACTTAGCAAAGGCTCTAAATGACTTTAGCAACTCAGGTGCTGATGTTCTCTA |
| | | CCAACCACCACAGACATCAATCACTTCTGCTGTTCTGCAGAGTGGTTTTAGGAAAATG |
| | | GCATTCCCGTCAGGCAAAGTTGAAGGGTGCATGGTACAAGTAACCTGTGGAACTACA |
| | | ACTCTTAATGGATTGTGGTTGGATGACACAGTATACTGTCCAAGACATGTCATTTGCA |
| | | CAGCAGAAGACATGCTTAATCCTAACTATGAAGATCTGCTCATTCGCAAATCCAACCA |
| | | TAGCTTTCTTGTTCAGGCTGGCAATGTTCAACTTCGTGTTATTGGCCATTCTATGCAA |
| | | AATTGTCTGCTTAGGCTTAAAGTTGATACTTCTAACCCTAAGACACCCAAGTATAAATT |
| | | TGTCCGTATCCAACCTGGTCAAACATTTTCAGTTCTAGCATGCTACAATGGTTCACCA |
| | | TCTGGTGTTTATCAGTGTGCCATGAGACCTAATCATACCATTAAAGGTTCTTTCCTTAA |
| | | TGGATCATGTGGTAGTGTTGGTTTTAACATTGATTATGATTGCGTGTCTTTCTGCTATA |
| | | TGCATCATATGGAGCTTCCAACAGGAGTACACGCTGGTACTGACTTAGAAGGTAAAT |
| | | TCTATGGTCCATTTGTTGACAGACAAACTGCACAGGCTGCAGGTACAGACACAACCA |
| | | TAACATTAAATGTTTTGGCATGGCTGTATGCTGCTGTTATCAATGGTGATAGGTGGTT |
| | | TCTTAATAGATTCACCACTACTTTGAATGACTTTAACCTTGTGGCAATGAAGTACAACT |
| | | ATGAACCTTTGACACAAGATCATGTTGACATATTGGGACCTCTTTCTGCTCAAACAGG |
| | | AATTGCCGTCTTAGATATGTGTGCTGCTTTGAAAGAGCTGCTGCAGAATGGTATGAAT |
| | | GGTCGTACTATCCTTGGTAGCACTATTTTAGAAGATGAGTTTACACCATTTGATGTTG |
| | | TTAGACAATGCTCTGGTGTTACCTTCCAAGGTAAGTTCAAGAAAATTGTTAAGGGCAC |
| | | TCATCATTGGATGCTTTTAACTTTCTTGACATCACTATTGATTCTTGTTCAAAGTACAC |
| | | AGTGGTCACTGTTTTTCTTTGTTTACGAGAATGCTTTCTTGCCATTTACTCTTGGTATT |
| | | ATGGCAATTGCTGCATGTGCTATGCTGCTTGTTAAGCATAAGCACGCATTCTTGTGCT |
| | | TGTTTCTGTTACCTTCTCTTGCAACAGTTGCTTACTTTAATATGGTCTACATGCCTGCT |
| | | AGCTGGGTGATGCGTATCATGACATGGCTTGAATTGGCTGACACTAGCTTGTCTGGT |
| | | TATAGGCTTAAGGATTGTGTTATGTATGCTTCAGCTTTAGTTTTGCTTATTCTCATGAC |
| | | AGCTCGCACTGTTTATGATGATGCTGCTAGACGTGTTTGGACACTGATGAATGTCATT |
| | | ACACTTGTTTACAAAGTCTACTATGGTAATGCTTTAGATCAAGCTATTTCCATGTGGG |
| | | CCTTAGTTATTTCTGTAACCTCTAACTATTCTGGTGTCGTTACGACTATCATGTTTTTA |
| | | GCTAGAGCTATAGTGTTTGTGTGTGTTGAGTATTACCCATTGTTATTTATTACTGGCAA |
| | | CACCTTACAGTGTATCATGCTTGTTTATTGTTTCTTAGGCTATTGTTGCTGCTGCTACT |
| | | TTGGCCTTTTCTGTTTACTCAACCGTTACTTCAGGCTTACTCTTGGTGTTTATGACTAC |
| | | TTGGTCTCTACACAAGAATTTAGGTATATGAACTCCCAGGGGCTTTTGCCTCCTAAGA |
| | | GTAGTATTGATGCTTTCAAGCTTAACATTAAGTTGTTGGGTATTGGAGGTAAACCATG |
| | | TATCAAGGTTGCTACTGTACAGTCTAAAATGTCTGACGTAAAGTGCACATCTGTGGTA |
| | | CTGCTCTCGGTTCTTCAACAACTTAGAGTAGAGTCATCTTCTAAATTGTGGGCACAAT |
| | | GTGTACAACTCCACAATGATATTCTTCTTGCAAAAGACACAACTGAAGCTTTCGAGAA |
| | | GATGGTTTCTCTTTTGTCTGTTTTGCTATCCATGCAGGGTGCTGTAGACATTAATAGG |
| | | TTGTGCGAGGAAATGCTCGATAACCGTGCTACTCTTCAGGCTATTGCTTCAGAATTTA |
| | | GTTCTTTACCATCATATGCCGCTTATGCCACTGCCCAGGAGGCCTATGAGCAGGCTG |
| | | TAGCTAATGGTGATTCTGAAGTCGTTCTCAAAAAGTTAAAGAAATCTTTGAATGTGGC |
| | | TAAATCTGAGTTTGACCGTGATGCTGCCATGCAACGCAAGTTGGAAAAGATGGCAGA |
| | | TCAGGCTATGACCCAAATGTACAAACAGGCAAGATCTGAGGACAAGAGGGCAAAAGT |
| | | AACTAGTGCTATGCAAACAATGCTCTTCACTATGCTTAGGAAGCTTGATAATGATGCA |
| | | CTTAACAACATTATCAACAATGCGCGTGATGGTTGTGTTCCACTCAACATCATACCAT |
| | | TGACTACAGCAGCCAAACTCATGGTTGTTGTCCCTGATTATGGTACCTACAAGAACAC |
| | | TTGTGATGGTAACACCTTTACATATGCATCTGCACTCTGGGAAATCCAGCAAGTTGTT |
| | | GATGCGGATAGCAAGATTGTTCAACTTAGTGAAATTAACATGGACAATTCACCAAATT |
| | | TGGCTTGGCCTCTTATTGTTACAGCTCTAAGAGCCAACTCAGCTGTTAAACTACAGAA |
| | | TAATGAACTGAGTCCAGTAGCACTACGACAGATGTCCTGTGCGGCTGGTACCACACA |
| | | AACAGCTTGTACTGATGACAATGCACTTGCCTACTATAACAATTCGAAGGGAGGTAG |
| | | GTTTGTGCTGGCATTACTATCAGACCACCAAGATCTCAAATGGGCTAGATTCCCTAAG |
| | | AGTGATGGTACAGGTACAATTTACACAGAACTGGAACCACCTTGTAGGTTTGTTACAG |
| | | ACACACCAAAAGGGCCTAAAGTGAAATACTTGTACTTCATCAAAGGCTTAAACAACCT |
| | | AAATAGAGGTATGGTGCTGGGCAGTTTAGCTGCTACAGTACGTCTTCAGGCTGGAAA |
| | | TGCTACAGAAGTACCTGCCAATTCAACTGTGCTTTCCTTCTGTGCTTTTGCAGTAGAC |
| | | CCTGCTAAAGCATATAAGGATTACCTAGCAAGTGGAGGACAACCAATCACCAACTGT |
| | | GTGAAGATGTTGTGTACACACACTGGTACAGGACAGGCAATTACTGTAACACCAGAA |
| | | GCTAACATGGACCAAGAGTCCTTTGGTGGTGCTTCATGTTGTCTGTATTGTAGATGC |
| | | CACATTGACCATCCAAATCCTAAAGGATTCTGTGACTTGAAAGGTAAGTACGTCCAAA |
| | | TACCTACCACTTGTGCTAATGACCCAGTGGGTTTTACACTTAGAAACACAGTCTGTAC |
| | | CGTCTGCGGAATGTGGAAAGGTTATGGCTGTAGTTGTGACCAACTCCGCGAACCCTT |
| | | GATGCAGTCTGCGGATGCATCAACGTTTTTAAACGGGTTTGCGGTGTAAGTGCAGCC |
| | | CGTCTTACACCGTGCGGCACAGGCACTAGTACTGATGTCGTCTACAGGGCTTTTGAT |
| | | ATTTACAACGAAAAAGTTGCTGGTTTTGCAAAGTTCCTAAAAACTAATTGCTGTCGCTT |
| | | CCAGGAGAAGGATGAGGAAGGCAATTTATTAGACTCTTACTTTGTAGTTAAGAGGCA |
| | | TACTATGTCTAACTACCAACATGAAGAGACTATTTATAACTTGGTTAAAGATTGTCCAG |
| | | CGGTTGCTGTCCATGACTTTTTCAAGTTTAGAGTAGATGGTGACATGGTACCACATAT |
| | | ATCACGTCAGCGTCTAACTAAATACACAATGGCTGATTTAGTCTATGCTCTACGTCAT |
| | | TTTGATGAGGGTAATTGTGATACATTAAAAGAAATACTCGTCACATACAATTGCTGTG |
| | | ATGATGATTATTTCAATAAGAAGGATTGGTATGACTTCGTAGAGAATCCTGACATCTT |
| | | ACGCGTATATGCTAACTTAGGTGAGCGTGTACGCCAATCATTATTAAAGACTGTACAA |
| | | TTCTGCGATGCTATGCGTGATGCAGGCATTGTAGGCGTACTGACATTAGATAATCAG |
| | | GATCTTAATGGGAACTGGTACGATTTCGGTGATTTCGTACAAGTAGCACCAGGCTGC |
| | | GGAGTTCCTATTGTGGATTCATATTACTCATTGCTGATGCCCATCCTCACTTTGACTA |
| | | GGGCATTGGCTGCTGAGTCCCATATGGATGCTGATCTCGCAAAACCACTTATTAAGT |
| | | GGGATTTGCTGAAATATGATTTTACGGAAGAGAGACTTTGTCTCTTCGACCGTTATTT |
| | | TAAATATTGGGACCAGACATACCATCCCAATTGTATTAACTGTTTGGATGATAGGTGT |
| | | ATCCTTCATTGTGCAAACTTTAATGTGTTATTTTCTACTGTGTTTCCACCTACAAGTTTT |
| | | GGACCACTAGTAAGAAAAATATTTGTAGATGGTGTTCCTTTTGTTGTTTCAACTGGAT |
| | | ACCATTTTCGTGAGTTAGGAGTCGTACATAATCAGGATGTAAACTTACATAGCTCGCG |
| | | TCTCAGTTTCAAGGAACTTTTAGTGTATGCTGCTGATCCAGCTATGCATGCAGCTTCT |
| | | GGCAATTTATTGCTAGATAAACGCACTACATGCTTTTCAGTAGCTGCACTAACAAACA |
| | | ATGTTGCTTTTCAAACTGTCAAACCCGGTAATTTTAATAAAGACTTTTATGACTTTGCT |
| | | GTGTCTAAAGGTTTCTTTAAGGAAGGAAGTTCTGTTGAACTAAAACACTTCTTCTTTG |
| | | CTCAGGATGGCAACGCTGCTATCAGTGATTATGACTATTATCGTTATAATCTGCCAAC |
| | | AATGTGTGATATCAGACAACTCCTATTCGTAGTTGAAGTTGTTGATAAATACTTTGATT |
| | | GTTACGATGGTGGCTGTATTAATGCCAACCAAGTAATCGTTAACAATCTGGATAAATC |
| | | AGCTGGTTTCCCATTTAATAAATGGGGTAAGGCTAGACTTTATTATGACTCAATGAGT |
| | | TATGAGGATCAAGATGCACTTTTCGCGTATACTAAGCGTAATGTCATCCCTACTATAA |
| | | CTCAAATGAATCTTAAGTATGCCATTAGTGCAAAGAATAGAGCTCGCACCGTAGCTG |
| | | GTGTCTCTATCTGTAGTACTATGACAAATAGACAGTTTCATCAGAAATTATTGAAGTCA |
| | | ATAGCCGCCACTAGAGGAGCTACTGTGGTAATTGGAACAAGCAAGTTTTACGGTGGC |
| | | TGGCATAATATGTTAAAAACTGTTTACAGTGATGTAGAAACTCCACACCTTATGGGTT |
| | | GGGATTATCCAAAATGTGACAGAGCCATGCCTAACATGCTTAGGATAATGGCCTCTC |
| | | TTGTTCTTGCTCGCAAACATAACACTTGCTGTAACTTATCACACCGTTTCTACAGGTTA |
| | | GCTAACGAGTGTGCGCAAGTATTAAGTGAGATGGTCATGTGTGGCGGCTCACTATAT |
| | | GTTAAACCAGGTGGAACATCATCCGGTGATGCTACAACTGCTTATGCTAATAGTGTCT |
| | | TTAACATTTGTCAAGCTGTTACAGCCAATGTAAATGCACTTCTTTCAACTGATGGTAAT |
| | | AAGATAGCTGACAAGTATGTCCGCAATCTACAACACAGGCTCTATGAGTGTCTCTATA |
| | | GAAATAGGGATGTTGATCATGAATTCGTGGATGAGTTTTACGCTTACCTGCGTAAACA |
| | | TTTCTCCATGATGATTCTTTCTGATGATGCCGTTGTGTGCTATAACAGTAACTATGCG |
| | | GCTCAAGGTTTAGTAGCTAGCATTAAGAACTTTAAGGCAGTTCTTTATTATCAAAATAA |
| | | TGTGTTCATGTCTGAGGCAAAATGTTGGACTGAGACTGACCTTACTAAAGGACCTCA |
| | | CGAATTTTGCTCACAGCATACAATGCTAGTTAAACAAGGAGATGATTACGTGTACCTG |
| | | CCTTACCCAGATCCATCAAGAATATTAGGCGCAGGCTGTTTTGTCGATGATATTGTCA |
| | | AAACAGATGGTACACTTATGATTGAAAGGTTCGTGTCACTGGCTATTGATGCTTACCC |
| | | ACTTACAAAACATCCTAATCAGGAGTATGCTGATGTCTTTCACTTGTATTTACAATACA |
| | | TTAGAAAGTTACATGATGAGCTTACTGGCCACATGTTGGACATGTATTCCGTAATGCT |
| | | AACTAATGATAACACCTCACGGTACTGGGAACCTGAGTTTTATGAGGCTATGTACACA |
| | | CCACATACAGTCTTGCAGGCTGTAGGTGCTTGTGTATTGTGCAATTCACAGACTTCAC |
| | | TTCGTTGCGGTGCCTGTATTAGGAGACCATTCCTATGTTGCAAGTGCTGCTATGACC |
| | | ATGTCATTTCAACATCACACAAATTAGTGTTGTCTGTTAATCCCTATGTTTGCAATGCC |
| | | CCAGGTTGTGATGTCACTGATGTGACACAACTGTATCTAGGAGGTATGAGCTATTATT |
| | | GCAAGTCACATAAGCCTCCCATTAGTTTTCCATTATGTGCTAATGGTCAGGTTTTTGG |
| | | TTTATACAAAAACACATGTGTAGGCAGTGACAATGTCACTGACTTCAATGCGATAGCA |
| | | ACATGTGATTGGACTAATGCTGGCGATTACATACTTGCCAACACTTGTACTGAGAGAC |
| | | TCAAGCTTTTCGCAGCAGAAACGCTCAAAGCCACTGAGGAAACATTTAAGCTGTCAT |
| | | ATGGTATTGCCACTGTACGCGAAGTACTCTCTGACAGAGAATTGCATCTTTCATGGG |
| | | AGGTTGGAAAACCTAGACCACCATTGAACAGAAACTATGTCTTTACTGGTTACCGTGT |
| | | AACTAAAAATAGTAAAGTACAGATTGGAGAGTACACCTTTGAAAAAGGTGACTATGGT |
| | | GATGCTGTTGTGTACAGAGGTACTACGACATACAAGTTGAATGTTGGTGATTACTTTG |
| | | TGTTGACATCTCACACTGTAATGCCACTTAGTGCACCTACTCTAGTGCCACAAGAGCA |
| | | CTATGTGAGAATTACTGGCTTGTACCCAACACTCAACATCTCAGATGAGTTTTCTAGC |
| | | AATGTTGCAAATTATCAAAAGGTCGGCATGCAAAAGTACTCTACACTCCAAGGACCAC |
| | | CTGGTACTGGTAAGAGTCATTTTGCCATCGGACTTGCTCTCTATTACCCATCTGCTCG |
| | | CATAGTGTATACGGCATGCTCTCATGCAGCTGTTGATGCCCTATGTGAAAAGGCATT |
| | | AAAATATTTGCCCATAGATAAATGTAGTAGAATCATACCTGCGCGTGCGCGCGTAGA |
| | | GTGTTTTGATAAATTCAAAGTGAATTCAACACTAGAACAGTATGTTTTCTGCACTGTAA |
| | | ATGCATTGCCAGAAACAACTGCTGACATTGTAGTCTTTGATGAAATCTCTATGGCTAC |
| | | TAATTATGACTTGAGTGTTGTCAATGCTAGACTTCGTGCAAAACACTACGTCTATATT |
| | | GGCGATCCTGCTCAATTACCAGCCCCCCGCACATTGCTGACTAAAGGCACACTAGAA |
| | | CCAGAATATTTTAATTCAGTGTGCAGACTTATGAAAACAATAGGTCCAGACATGTTCC |
| | | TTGGAACTTGTCGCCGTTGTCCTGCTGAAATTGTTGACACTGTGAGTGCTTTAGTTTA |
| | | TGACAATAAGCTAAAAGCACACAAGGATAAGTCAGCTCAATGCTTCAAAATGTTCTAC |
| | | AAAGGTGTTATTACACATGATGTTTCATCTGCAATCAACAGACCTCAAATAGGCGTTG |
| | | TAAGAGAATTTCTTACACGCAATCCTGCTTGGAGAAAAGCTGTTTTTATCTCACCTTAT |
| | | AATTCACAGAACGCTGTAGCTTCAAAAATCTTAGGATTGCCTACGCAGACTGTTGATT |
| | | CATCACAGGGTTCTGAATATGACTATGTCATATTCACACAAACTACTGAAACAGCACA |
| | | CTCTTGTAATGTCAACCGCTTCAATGTGGCTATCACAAGGGCAAAAATTGGCATTTTG |
| | | TGCATAATGTCTGATAGAGATCTTTATGACAAACTGCAATTTACAAGTCTAGAAATACC |
| | | ACGTCGCAATGTGGCTACATTACAAGCAGAAAATGTAACTGGACTTTTTAAGGACTGT |
| | | AGTAAGATCATTACTGGTCTTCATCCTACACAGGCACCTACACACCTCAGCGTTGATA |
| | | TAAAGTTCAAGACTGAAGGATTATGTGTTGACATACCAGGCATACCAAAGGACATGA |
| | | CCTACCGTAGACTCATCTCTATGATGGGTTTCAAAATGAATTACCAAGTCAATGGTTA |
| | | CCCTAATATGTTTATCACCCGCGAAGAAGCTATTCGTCACGTTCGTGCGTGGATTGG |
| | | CTTTGATGTAGAGGGCTGTCATGCAACTAGAGATGCTGTGGGTACTAACCTACCTCT |
| | | CCAGCTAGGATTTTCTACAGGTGTTAACTTAGTAGCTGTACCGACTGGTTATGTTGAC |
| | | ACTGAAAATAACACAGAATTCACCAGAGTTAATGCAAAACCTCCACCAGGTGACCAG |
| | | TTTAAACATCTTATACCACTCATGTATAAAGGCTTGCCCTGGAATGTAGTGCGTATTA |
| | | AGATAGTACAAATGCTCAGTGATACACTGAAAGGATTGTCAGACAGAGTCGTGTTCG |
| | | TCCTTTGGGCGCATGGCTTTGAGCTTACATCAATGAAGTACTTTGTCAAGATTGGACC |
| | | TGAAAGAACGTGTTGTCTGTGTGACAAACGTGCAACTTGCTTTTCTACTTCATCAGAT |
| | | ACTTATGCCTGCTGGAATCATTCTGTGGGTTTTGACTATGTCTATAACCCATTTATGAT |
| | | TGATGTTCAGCAGTGGGGCTTTACGGGTAACCTTCAGAGTAACCATGACCAACATTG |
| | | CCAGGTACATGGAAATGCACATGTGGCTAGTTGTGATGCTATCATGACTAGATGTTTA |
| | | GCAGTCCATGAGTGCTTTGTTAAGCGCGTTGATTGGTCTGTTGAATACCCTATTATAG |
| | | GAGATGAACTGAGGGTTAATTCTGCTTGCAGAAAAGTACAACACATGGTTGTGAAGT |
| | | CTGCATTGCTTGCTGATAAGTTTCCAGTTCTTCATGACATTGGAAATCCAAAGGCTAT |
| | | CAAGTGTGTGCCTCAGGCTGAAGTAGAATGGAAGTTCTACGATGCTCAGCCATGTAG |
| | | TGACAAAGCTTACAAAATAGAGGAACTCTTCTATTCTTATGCTACACATCACGATAAAT |
| | | TCACTGATGGTGTTTGTTTGTTTTGGAATTGTAACGTTGATCGTTACCCAGCCAATGC |
| | | AATTGTGTGTAGGTTTGACACAAGAGTCTTGTCAAACTTGAACTTACCAGGCTGTGAT |
| | | GGTGGTAGTTTGTATGTGAATAAGCATGCATTCCACACTCCAGCTTTCGATAAAAGTG |
| | | CATTTACTAATTTAAAGCAATTGCCTTTCTTTTACTATTCTGATAGTCCTTGTGAGTCT |
| | | CATGGCAAACAAGTAGTGTCGGATATTGATTATGTTCCACTCAAATCTGCTACGTGTA |
| | | TTACACGATGCAATTTAGGTGGTGCTGTTTGCAGACACCATGCAAATGAGTACCGAC |
| | | AGTACTTGGATGCATATAATATGATGATTTCTGCTGGATTTAGCCTATGGATTTACAAA |
| | | CAATTTGATACTTATAACCTGTGGAATACATTTACCAGGTTACAGAGTTTAGAAAATGT |
| | | GGCTTATAATGTTGTTAATAAAGGACACTTTGATGGACACGCCGGCGAAGCACCTGT |
| | | TTCCATCATTAATAATGCTGTTTACACAAAGGTAGATGGTATTGATGTGGAGATCTTT |
| | | GAAAATAAGACAACACTTCCTGTTAATGTTGCATTTGAGCTTTGGGCTAAGCGTAACA |
| | | TTAAACCAGTGCCAGAGATTAAGATACTCAATAATTTGGGTGTTGATATCGCTGCTAA |
| | | TACTGTAATCTGGGACTACAAAAGAGAAGCCCCAGCACATGTATCTACAATAGGTGT |
| | | CTGCACAATGACTGACATTGCCAAGAAACCTACTGAGAGTGCTTGTTCTTCACTTACT |
| | | GTCTTGTTTGATGGTAGAGTGGAAGGACAGGTAGACCTTTTTAGAAACGCCCGTAAT |
| | | GGTGTTTTAATAACAGAAGGTTCAGTCAAAGGTCTAACACCTTCAAAGGGACCAGCA |
| | | CAAGCTAGCGTCAATGGAGTCACATTAATTGGAGAATCAGTAAAAACACAGTTTAACT |
| | | ACTTTAAGAAAGTAGACGGCATTATTCAACAGTTGCCTGAAACCTACTTTACTCAGAG |
| | | CAGAGACTTAGAGGATTTTAAGCCCAGATCACAAATGGAAACTGACTTTCTCGAGCT |
| | | CGCTATGGATGAATTCATACAGCGATATAAGCTCGAGGGCTATGCCTTCGAACACAT |
| | | CGTTTATGGAGATTTCAGTCATGGACAACTTGGCGGTCTTCATTTAATGATAGGCTTA |
| | | GCCAAGCGCTCACAAGATTCACCACTTAAATTAGAGGATTTTATCCCTATGGACAGCA |
| | | CAGTGAAAAATTACTTCATAACAGATGCGCAAACAGGTTCATCAAAATGTGTGTGTTC |
| | | TGTGATTGATCTTTTACTTGATGACTTTGTCGAGATAATAAAGTCACAAGATTTGTCAG |
| | | TGATTTCAAAAGTGGTCAAGGTTACAATTGACTATGCTGAAATTTCATTCATGCTTTGG |
| | | TGTAAGGATGGACATGTTGAAACCTTCTACCCAAAACTACAAGCAAGTCAAGCGTGG |
| | | CAACCAGGTGTTGCGATGCCTAACTTGTACAAGATGCAAAGAATGCTTCTTGAAAAGT |
| | | GTGACCTTCAGAATTATGGTGAAAATGCTGTTATACCAAAAGGAATAATGATGAATGT |
| | | CGCAAAGTATACTCAACTGTGTCAATACTTAAATACACTTACTTTAGCTGTACCCTACA |
| | | ACATGAGAGTTATTCACTTTGGTGCTGGCTCTGATAAAGGAGTTGCACCAGGTACAG |
| | | CTGTGCTCAGACAATGGTTGCCAACTGGCACACTACTTGTCGATTCAGATCTTAATGA |
| | | CTTCGTCTCCGACGCAGATTCTACTTTAATTGGAGACTGTGCAACAGTACATACGGCT |
| | | AATAAATGGGACCTTATTATTAGCGATATGTATGACCCTAGGACCAAACATGTGACAA |
| | | AAGAGAATGACTCTAAAGAAGGGTTTTTCACTTATCTGTGTGGATTTATAAAGCAAAA |
| | | ACTAGCCCTGGGTGGTTCTATAGCTGTAAAGATAACAGAGCATTCTTGGAATGCTGA |
| | | CCTTTACAAGCTTATGGGCCATTTCTCATGGTGGACAGCTTTTGTTACAAATGTAAAT |
| | | GCATCATCATCGGAAGCATTTTTAATTGGGGCTAACTATCTTGGCAAGCCGAAGGAA |
| | | CAAATTGATGGCTATACCATGCATGCTAACTACATTTTCTGGAGGAACACAAATCCTA |
| | | TCCAGTTGTCTTCCTATTCACTCTTTGACATGAGCAAATTTCCTCTTAAATTAAGAGGA |
| | | ACTGCTGTAATGTCTCTTAAGGAGAATCAAATCAATGATATGATTTATTCTCTTCTGGA |
| | | AAAAGGTAGGCTTATCATTAGAGAAAACAACAGAGTTGTGGTTTCAAGTGATATTCTT |
| | | GTTAACAACTAAACGAACATGTTTATTTTCTTATTATTTCTTACTCTCACTAGTGGTAGT |
| | | GACCTTGACCGGTGCACCACTTTTGATGATGTTCAAGCTCCTAATTACACTCAACATA |
| | | CTTCATCTATGAGGGGGGTTTACTATCCTGATGAAATTTTTAGATCAGACACTCTTTAT |
| | | TTAACTCAGGATTTATTTCTTCCATTTTATTCTAATGTTACAGGGTTTCATACTATTAAT |
| | | CATACGTTTGGCAACCCTGTCATACCTTTTAAGGATGGTATTTATTTTGCTGCCACAG |
| | | AGAAATCAAATGTTGTCCGTGGTTGGGTTTTTGGTTCTACCATGAACAACAAGTCACA |
| | | GTCGGTGATTATTATTAACAATTCTACTAATGTTGTTATACGAGCATGTAACTTTGAAT |
| | | TGTGTGACAACCCTTTCTTTGCTGTTTCTAAACCCATGGGTACACAGACACATACTAT |
| | | GATATTCGATAATGCATTTAATTGCACTTTCGAGTACATATCTGATGCCTTTTCGCTTG |
| | | ATGTTTCAGAAAAGTCAGGTAATTTTAAACACTTACGAGAGTTTGTGTTTAAAAATAAA |
| | | GATGGGTTTCTCTATGTTTATAAGGGCTATCAACCTATAGATGTAGTTCGTGATCTAC |
| | | CTTCTGGTTTTAACACTTTGAAACCTATTTTTAAGTTGCCTCTTGGTATTAACATTACA |
| | | AATTTTAGAGCCATTCTTACAGCCTTTTCACCTGCTCAAGACATTTGGGGCACGTCAG |
| | | CTGCAGCCTATTTTGTTGGCTATTTAAAGCCAACTACATTTATGCTCAAGTATGATGAA |
| | | AATGGTACAATCACAGATGCTGTTGATTGTTCTCAAAATCCACTTGCTGAACTCAAAT |
| | | GCTCTGTTAAGAGCTTTGAGATTGACAAAGGAATTTACCAGACCTCTAATTTCAGGGT |
| | | TGTTCCCTCAGGAGATGTTGTGAGATTCCCTAATATTACAAACTTGTGTCCTTTTGGA |
| | | GAGGTTTTTAATGCTACTAAATTCCCTTCTGTCTATGCATGGGAGAGAAAAAAAATTT |
| | | CTAATTGTGTTGCTGATTACTCTGTGCTCTACAACTCAACATTTTTTTCAACCTTTAAG |
| | | TGCTATGGCGTTTCTGCCACTAAGTTGAATGATCTTTGCTTCTCCAATGTCTATGCAG |
| | | ATTCTTTTGTAGTCAAGGGAGATGATGTAAGACAAATAGCGCCAGGACAAACTGGTG |
| | | TTATTGCTGATTATAATTATAAATTGCCAGATGATTTCATGGGTTGTGTCCTTGCTTGG |
| | | AATACTAGGAACATTGATGCTACTTCAACTGGTAATTATAATTATAAATATAGGTATCT |
| | | TAGACATGGCAAGCTTAGGCCCTTTGAGAGAGACATATCTAATGTGCCTTTCTCCCCT |
| | | GATGGCAAACCTTGCACCCCACCTGCTCTTAATTGTTATTGGCCATTAAATGATTATG |
| | | GTTTTTACACCACTACTGGCATTGGCTACCAACCTTACAGAGTTGTAGTACTTTCTTTT |
| | | GAACTTTTAAATGCACCGGCCACGGTTTGTGGACCAAAATTATCCACTGACCTTATTA |
| | | AGAACCAGTGTGTCAATTTTAATTTTAATGGACTCACTGGTACTGGTGTGTTAACTCC |
| | | TTCTTCAAAGAGATTTCAACCATTTCAACAATTTGGCCGTGATGTTTCTGATTTCACTG |
| | | ATTCCGTTCGAGATCCTAAAACATCTGAAATATTAGACATTTCACCTTGCGCTTTTGG |
| | | GGGTGTAAGTGTAATTACACCTGGAACAAATGCTTCATCTGAAGTTGCTGTTCTATAT |
| | | CAAGATGTTAACTGCACTGATGTTTCTACAGCAATTCATGCAGATCAACTCACACCAG |
| | | CTTGGCGCATATATTCTACTGGAAACAATGTATTCCAGACTCAAGCAGGCTGTCTTAT |
| | | AGGAGCTGAGCATGTCGACACTTCTTATGAGTGCGACATTCCTATTGGAGCTGGCAT |
| | | TTGTGCTAGTTACCATACAGTTTCTTTATTACGTAGTACTAGCCAAAAATCTATTGTGG |
| | | CTTATACTATGTCTTTAGGTGCTGATAGTTCAATTGCTTACTCTAATAACACCATTGCT |
| | | ATACCTACTAACTTTTCAATTAGCATTACTACAGAAGTAATGCCTGTTTCTATGGCTAA |
| | | AACCTCCGTAGATTGTAATATGTACATCTGCGGAGATTCTACTGAATGTGCTAATTTG |
| | | CTTCTCCAATATGGTAGCTTTTGCACACAACTAAATCGTGCACTCTCAGGTATTGCTG |
| | | CTGAACAGGATCGCAACACACGTGAAGTGTTCGCTCAAGTCAAACAAATGTACAAAA |
| | | CCCCAACTTTGAAATATTTTGGTGGTTTTAATTTTTCACAAATATTACCTGACCCTCTA |
| | | AAGCCAACTAAGAGGTCTTTTATTGAGGACTTGCTCTTTAATAAGGTGACACTCGCTG |
| | | ATGCTGGCTTCATGAAGCAATATGGCGAATGCCTAGGTGATATTAATGCTAGAGATCT |
| | | CATTTGTGCGCAGAAGTTCAATGGACTTACAGTGTTGCCACCTCTGCTCACTGATGAT |
| | | ATGATTGCTGCCTACACTGCTGCTCTAGTTAGTGGTACTGCCACTGCTGGATGGACA |
| | | TTTGGTGCTGGCGCTGCTCTTCAAATACCTTTTGCTATGCAAATGGCATATAGGTTCA |
| | | ATGGCATTGGAGTTACCCAAAATGTTCTCTATGAGAACCAAAAACAAATCGCCAACCA |
| | | ATTTAACAAGGCGATTAGTCAAATTCAAGAATCACTTACAACAACATCAACTGCATTG |
| | | GGCAAGCTGCAAGACGTTGTTAACCAGAATGCTCAAGCATTAAACACACTTGTTAAAC |
| | | AACTTAGCTCTAATTTTGGTGCAATTTCAAGTGTGCTAAATGATATCCTTTCGCGACTT |
| | | GATAAAGTCGAGGCGGAGGTACAAATTGACAGGTTAATTACAGGCAGACTTCAAAGC |
| | | CTTCAAACCTATGTAACACAACAACTAATCAGGGCTGCTGAAATCAGGGCTTCTGCTA |
| | | ATCTTGCTGCTACTAAAATGTCTGAGTGTGTTCTTGGACAATCAAAAAGAGTTGACTT |
| | | TTGTGGAAAGGGCTACCACCTTATGTCCTTCCCACAAGCAGCCCCGCATGGTGTTGT |
| | | CTTCCTACATGTCACGTATGTGCCATCCCAGGAGAGGAACTTCACCACAGCGCCAGC |
| | | AATTTGTCATGAAGGCAAAGCATACTTCCCTCGTGAAGGTGTTTTTGTGTTTAATGGC |
| | | ACTTCTTGGTTTATTACACAGAGGAACTTCTTTTCTCCACAAATAATTACTACAGACAA |
| | | TACATTTGTCTCAGGAAATTGTGATGTCGTTATTGGCATCATTAACAACACAGTTTATG |
| | | ATCCTCTGCAACCTGAGCTTGACTCATTCAAAGAAGAGCTGGACAAGTACTTCAAAAA |
| | | TCATACATCACCAGATGTTGATCTTGGCGACATTTCAGGCATTAACGCTTCTGTCGTC |
| | | AACATTCAAAAAGAAATTGACCGCCTCAATGAGGTCGCTAAAAATTTAAATGAATCAC |
| | | TCATTGACCTTCAAGAATTGGGAAAATATGAGCAATATATTAAATGGCCTTGGTATGT |
| | | TTGGCTCGGCTTCATTGCTGGACTAATTGCCATCGTCATGGTTACAATCTTGCTTTGT |
| | | TGCATGACTAGTTGTTGCAGTTGCCTCAAGGGTGCATGCTCTTGTGGTTCTTGCTGC |
| | | AAGTTTGATGAGGATGACTCTGAGCCAGTTCTCAAGGGTGTCAAATTACATTACACAT |
| | | AAACGAACTTATGGATTTGTTTATGAGATTTTTTACTCTTAGATCAATTACTGCACAGC |
| | | CAGTAAAAATTGACAATGCTTCTCCTGCAAGTACTGTTCATGCTACAGCAACGATACC |
| | | GCTACAAGCCTCACTCCCTTTCGGATGGCTTGTTATTGGCGTTGCATTTCTTGCTGTT |
| | | TTTCAGAGCGCTACCAAAATAATTGCGCTCAATAAAAGATGGCAGCTAGCCCTTTATA |
| | | AGGGCTTCCAGTTCATTTGCAATTTACTGCTGCTATTTGTTACCATCTATTCACATCTT |
| | | TTGCTTGTCGCTGCAGGTATGGAGGCGCAATTTTTGTACCTCTATGCCTTGATATATT |
| | | TTCTACAATGCATCAACGCATGTAGAATTATTATGAGATGTTGGCTTTGTTGGAAGTG |
| | | CAAATCCAAGAACCCATTACTTTATGATGCCAACTACTTTGTTTGCTGGCACACACAT |
| | | AACTATGACTACTGTATACCATATAACAGTGTCACAGATACAATTGTCGTTACTGAAG |
| | | GTGACGGCATTTCAACACCAAAACTCAAAGAAGACTACCAAATTGGTGGTTATTCTGA |
| | | GGATAGGCACTCAGGTGTTAAAGACTATGTCGTTGTACATGGCTATTTCACCGAAGTT |
| | | TACTACCAGCTTGAGTCTACACAAATTACTACAGACACTGGTATTGAAAATGCTACAT |
| | | TCTTCATCTTTAACAAGCTTGTTAAAGACCCACCGAATGTGCAAATACACACAATCGA |
| | | CGGCTCTTCAGGAGTTGCTAATCCAGCAATGGATCCAATTTATGATGAGCCGACGAC |
| | | GACTACTAGCGTGCCTTTGTAAGCACAAGAAAGTGAGTACGAACTTATGTACTCATTC |
| | | GTTTCGGAAGAAACAGGTACGTTAATAGTTAATAGCGTACTTCTTTTTCTTGCTTTCGT |
| | | GGTATTCTTGCTAGTCACACTAGCCATCCTTACTGCGCTTCGATTGTGTGCGTACTGC |
| | | TGCAATATTGTTAACGTGAGTTTAGTAAAACCAACGGTTTACGTCTACTCGCGTGTTA |
| | | AAAATCTGAACTCTTCTGAAGGAGTTCCTGATCTTCTGGTCTAAACGAACTAACTATT |
| | | ATTATTATTCTGTTTGGAACTTTAACATTGCTTATCATGGCAGACAACGGTACTATTAC |
| | | CGTTGAGGAGCTTAAACAACTCCTGGAACAATGGAACCTAGTAATAGGTTTCCTATTC |
| | | CTAGCCTGGATTATGTTACTACAATTTGCCTATTCTAATCGGAACAGGTTTTTGTACAT |
| | | AATAAAGCTTGTTTTCCTCTGGCTCTTGTGGCCAGTAACACTTGCTTGTTTTGTGCTT |
| | | GCTGCTGTCTACAGAATTAATTGGGTGACTGGCGGGATTGCGATTGCAATGGCTTGT |
| | | ATTGTAGGCTTGATGTGGCTTAGCTACTTCGTTGCTTCCTTCAGGCTGTTTGCTCGTA |
| | | CCCGCTCAATGTGGTCATTCAACCCAGAAACAAACATTCTTCTCAATGTGCCTCTCCG |
| | | GGGGACAATTGTGACCAGACCGCTCATGGAAAGTGAACTTGTCATTGGTGCTGTGAT |
| | | CATTCGTGGTCACTTGCGAATGGCCGGACACTCCCTAGGGCGCTGTGACATTAAGG |
| | | ACCTGCCAAAAGAGATCACTGTGGCTACATCACGAACGCTTTCTTATTACAAATTAGG |
| | | AGCGTCGCAGCGTGTAGGCACTGATTCAGGTTTTGCTGCATACAACCGCTACCGTAT |
| | | TGGAAACTATAAATTAAATACAGACCACGCCGGTAGCAACGACAATATTGCTTTGCTA |
| | | GTACAGTAAGTGACAACAGATGTTTCATCTTGTTGACTTCCAGGTTACAATAGCAGAG |
| | | ATATTGATTATCATTATGAGGACTTTCAGGATTGCTATTTGGAATCTTGACGTTATAAT |
| | | AAGTTCAATAGTGAGACAATTATTTAAGCCTCTAACTAAGAAGAATTATTCGGAGTTA |
| | | GATGATGAAGAACCTATGGAGTTAGATTATCCATAAAACGAACATGAAAATTATTCTC |
| | | TTCCTGACATTGATTGTATTTACATCTTGCGAGCTATATCACTATCAGGAGTGTGTTA |
| | | GAGGTACGACTGTACTACTAAAAGAACCTTGCCCATCAGGAACATACGAGGGCAATT |
| | | CACCATTTCACCCTCTTGCTGACAATAAATTTGCACTAACTTGCACTAGCACACACTT |
| | | TGCTTTTGCTTGTGCTGACGGTACTCGACATACCTATCAGCTGCGTGCAAGATCAGT |
| | | TTCACCAAAACTTTTCATCAGACAAGAGGAGGTTCAACAAGAGCTCTACTCGCCACTT |
| | | TTTCTCATTGTTGCTGCTCTAGTATTTTTAATACTTTGCTTCACCATTAAGAGAAAGAC |
| | | AGAATGAATGAGCTCACTTTAATTGACTTCTATTTGTGCTTTTTAGCCTTTCTGCTATT |
| | | CCTTGTTTTAATAATGCTTATTATATTTTGGTTTTCACTCGAAATCCAGGATCTAGAAG |
| | | AACCTTGTACCAAAGTCTAAACGAACATGAAACTTCTCATTGTTTTGACTTGTATTTCT |
| | | CTATGCAGTTGCATATGCACTGTAGTACAGCGCTGTGCATCTAATAAACCTCATGTGC |
| | | TTGAAGATCCTTGTAAGGTACAACACTAGGGGTAATACTTATAGCACTGCTTGGCTTT |
| | | GTGCTCTAGGAAAGGTTTTACCTTTTCATAGATGGCACACTATGGTTCAAACATGCAC |
| | | ACCTAATGTTACTATCAACTGTCAAGATCCAGCTGGTGGTGCGCTTATAGCTAGGTGT |
| | | TGGTACCTTCATGAAGGTCACCAAACTGCTGCATTTAGAGACGTACTTGTTGTTTTAA |
| | | ATAAACGAACAAATTAAAATGTCTGATAATGGACCCCAATCAAACCAACGTAGTGCCC |
| | | CCCGCATTACATTTGGTGGACCCACAGATTCAACTGACAATAACCAGAATGGAGGAC |
| | | GCAATGGGGCAAGGCCAAAACAGCGCCGACCCCAAGGTTTACCCAATAATACTGCG |
| | | TCTTGGTTCACAGCTCTCACTCAGCATGGCAAGGAGGAACTTAGATTCCCTCGAGGC |
| | | CAGGGCGTTCCAATCAACACCAATAGTGGTCCAGATGACCAAATTGGCTACTACCGA |
| | | AGAGCTACCCGACGAGTTCGTGGTGGTGACGGCAAAATGAAAGAGCTCAGCCCCAG |
| | | ATGGTACTTCTATTACCTAGGAACTGGCCCAGAAGCTTCACTTCCCTACGGCGCTAA |
| | | CAAAGAAGGCATCGTATGGGTTGCAACTGAGGGAGCCTTGAATACACCCAAAGACCA |
| | | CATTGGCACCCGCAATCCTAATAACAATGCTGCCACCGTGCTACAACTTCCTCAAGG |
| | | AACAACATTGCCAAAAGGCTTCTACGCAGAGGGAAGCAGAGGCGGCAGTCAAGCCT |
| | | CTTCTCGCTCCTCATCACGTAGTCGCGGTAATTCAAGAAATTCAACTCCTGGCAGCA |
| | | GTAGGGGAAATTCTCCTGCTCGAATGGCTAGCGGAGGTGGTGAAACTGCCCTCGCG |
| | | CTATTGCTGCTAGACAGATTGAACCAGCTTGAGAGCAAAGTTTCTGGTAAAGGCCAA |
| | | CAACAACAAGGCCAAACTGTCACTAAGAAATCTGCTGCTGAGGCATCTAAAAAGCCT |
| | | CGCCAAAAACGTACTGCCACAAAACAGTACAACGTCACTCAAGCATTTGGGAGACGT |
| | | GGTCCAGAACAAACCCAAGGAAATTTCGGGGACCAAGACCTAATCAGACAAGGAACT |
| | | GATTACAAACATTGGCCGCAAATTGCACAATTTGCTCCAAGTGCCTCTGCATTCTTTG |
| | | GAATGTCACGCATTGGCATGGAAGTCACACCTTCGGGAACATGGCTGACTTATCATG |
| | | GAGCCATTAAATTGGATGACAAAGATCCACAATTCAAAGACAACGTCATACTGCTGAA |
| | | CAAGCACATTGACGCATACAAAACATTCCCACCAACAGAGCCTAAAAAGGACAAAAA |
| | | GAAAAAGACTGATGAAGCTCAGCCTTTGCCGCAGAGACAAAAGAAGCAGCCCACTGT |
| | | GACTCTTCTTCCTGCGGCTGACATGGATGATTTCTCCAGACAACTTCAAAATTCCATG |
| | | AGTGGAGCTTCTGCTGATTCAACTCAGGCATAAACACTCATGATGACCACACAAGGC |
| | | AGATGGGCTATGTAAACGTTTTCGCAATTCCGTTTACGATACATAGTCTACTCTTGTG |
| | | CAGAATGAATTCTCGTAACTAAACAGCACAAGTAGGTTTAGTTAACTTTAATCTCACAT |
| | | AGCAATCTTTAATCAATGTGTAACATTAGGGAGGACTTGAAAGAGCCACCACATTTTC |
| | | ATCGAGGCCACGCGGAGTACGATCGAGGGTACAGTGAATAATGCTAGGGAGAGCTG |
| | | CCTATATGGAAGAGCCCTAATGTGTAAAATTAATTTTAGTAGTGCTATCCCCATGTGA |
| | | TTTTAATAGCTTCTTAGGAGAATGACAAAAAAAAAAAAAAAAAAAAAAAA |
| 8 | SARS-CoV-2 | ATTAAAGGTTTATACCTTCCCAGGTAACAAACCAACCAACTTTCGATCTCTTGTAGAT |
| | (GenBank: | CTGTTCTCTAAACGAACTTTAAAATCTGTGTGGCTGTCACTCGGCTGCATGCTTAGTG |
| | NC_045512.2) | CACTCACGCAGTATAATTAATAACTAATTACTGTCGTTGACAGGACACGAGTAACTCG |
| | | TCTATCTTCTGCAGGCTGCTTACGGTTTCGTCCGTGTTGCAGCCGATCATCAGCACA |
| | | TCTAGGTTTCGTCCGGGTGTGACCGAAAGGTAAGATGGAGAGCCTTGTCCCTGGTTT |
| | | CAACGAGAAAACACACGTCCAACTCAGTTTGCCTGTTTTACAGGTTCGCGACGTGCT |
| | | CGTACGTGGCTTTGGAGACTCCGTGGAGGAGGTCTTATCAGAGGCACGTCAACATC |
| | | TTAAAGATGGCACTTGTGGCTTAGTAGAAGTTGAAAAAGGCGTTTTGCCTCAACTTGA |
| | | ACAGCCCTATGTGTTCATCAAACGTTCGGATGCTCGAACTGCACCTCATGGTCATGT |
| | | TATGGTTGAGCTGGTAGCAGAACTCGAAGGCATTCAGTACGGTCGTAGTGGTGAGA |
| | | CACTTGGTGTCCTTGTCCCTCATGTGGGCGAAATACCAGTGGCTTACCGCAAGGTTC |
| | | TTCTTCGTAAGAACGGTAATAAAGGAGCTGGTGGCCATAGTTACGGCGCCGATCTAA |
| | | AGTCATTTGACTTAGGCGACGAGCTTGGCACTGATCCTTATGAAGATTTTCAAGAAAA |
| | | CTGGAACACTAAACATAGCAGTGGTGTTACCCGTGAACTCATGCGTGAGCTTAACGG |
| | | AGGGGCATACACTCGCTATGTCGATAACAACTTCTGTGGCCCTGATGGCTACCCTCT |
| | | TGAGTGCATTAAAGACCTTCTAGCACGTGCTGGTAAAGCTTCATGCACTTTGTCCGAA |
| | | CAACTGGACTTTATTGACACTAAGAGGGGTGTATACTGCTGCCGTGAACATGAGCAT |
| | | GAAATTGCTTGGTACACGGAACGTTCTGAAAAGAGCTATGAATTGCAGACACCTTTTG |
| | | AAATTAAATTGGCAAAGAAATTTGACACCTTCAATGGGGAATGTCCAAATTTTGTATTT |
| | | CCCTTAAATTCCATAATCAAGACTATTCAACCAAGGGTTGAAAAGAAAAAGCTTGATG |
| | | GCTTTATGGGTAGAATTCGATCTGTCTATCCAGTTGCGTCACCAAATGAATGCAACCA |
| | | AATGTGCCTTTCAACTCTCATGAAGTGTGATCATTGTGGTGAAACTTCATGGCAGACG |
| | | GGCGATTTTGTTAAAGCCACTTGCGAATTTTGTGGCACTGAGAATTTGACTAAAGAAG |
| | | GTGCCACTACTTGTGGTTACTTACCCCAAAATGCTGTTGTTAAAATTTATTGTCCAGC |
| | | ATGTCACAATTCAGAAGTAGGACCTGAGCATAGTCTTGCCGAATACCATAATGAATCT |
| | | GGCTTGAAAACCATTCTTCGTAAGGGTGGTCGCACTATTGCCTTTGGAGGCTGTGTG |
| | | TTCTCTTATGTTGGTTGCCATAACAAGTGTGCCTATTGGGTTCCACGTGCTAGCGCTA |
| | | ACATAGGTTGTAACCATACAGGTGTTGTTGGAGAAGGTTCCGAAGGTCTTAATGACA |
| | | ACCTTCTTGAAATACTCCAAAAAGAGAAAGTCAACATCAATATTGTTGGTGACTTTAAA |
| | | CTTAATGAAGAGATCGCCATTATTTTGGCATCTTTTTCTGCTTCCACAAGTGCTTTTGT |
| | | GGAAACTGTGAAAGGTTTGGATTATAAAGCATTCAAACAAATTGTTGAATCCTGTGGT |
| | | AATTTTAAAGTTACAAAAGGAAAAGCTAAAAAAGGTGCCTGGAATATTGGTGAACAGA |
| | | AATCAATACTGAGTCCTCTTTATGCATTTGCATCAGAGGCTGCTCGTGTTGTACGATC |
| | | AATTTTCTCCCGCACTCTTGAAACTGCTCAAAATTCTGTGCGTGTTTTACAGAAGGCC |
| | | GCTATAACAATACTAGATGGAATTTCACAGTATTCACTGAGACTCATTGATGCTATGA |
| | | TGTTCACATCTGATTTGGCTACTAACAATCTAGTTGTAATGGCCTACATTACAGGTGG |
| | | TGTTGTTCAGTTGACTTCGCAGTGGCTAACTAACATCTTTGGCACTGTTTATGAAAAA |
| | | CTCAAACCCGTCCTTGATTGGCTTGAAGAGAAGTTTAAGGAAGGTGTAGAGTTTCTTA |
| | | GAGACGGTTGGGAAATTGTTAAATTTATCTCAACCTGTGCTTGTGAAATTGTCGGTGG |
| | | ACAAATTGTCACCTGTGCAAAGGAAATTAAGGAGAGTGTTCAGACATTCTTTAAGCTT |
| | | GTAAATAAATTTTTGGCTTTGTGTGCTGACTCTATCATTATTGGTGGAGCTAAACTTAA |
| | | AGCCTTGAATTTAGGTGAAACATTTGTCACGCACTCAAAGGGATTGTACAGAAAGTGT |
| | | GTTAAATCCAGAGAAGAAACTGGCCTACTCATGCCTCTAAAAGCCCCAAAAGAAATTA |
| | | TCTTCTTAGAGGGAGAAACACTTCCCACAGAAGTGTTAACAGAGGAAGTTGTCTTGA |
| | | AAACTGGTGATTTACAACCATTAGAACAACCTACTAGTGAAGCTGTTGAAGCTCCATT |
| | | GGTTGGTACACCAGTTTGTATTAACGGGCTTATGTTGCTCGAAATCAAAGACACAGAA |
| | | AAGTACTGTGCCCTTGCACCTAATATGATGGTAACAAACAATACCTTCACACTCAAAG |
| | | GCGGTGCACCAACAAAGGTTACTTTTGGTGATGACACTGTGATAGAAGTGCAAGGTT |
| | | ACAAGAGTGTGAATATCACTTTTGAACTTGATGAAAGGATTGATAAAGTACTTAATGA |
| | | GAAGTGCTCTGCCTATACAGTTGAACTCGGTACAGAAGTAAATGAGTTCGCCTGTGT |
| | | TGTGGCAGATGCTGTCATAAAAACTTTGCAACCAGTATCTGAATTACTTACACCACTG |
| | | GGCATTGATTTAGATGAGTGGAGTATGGCTACATACTACTTATTTGATGAGTCTGGTG |
| | | AGTTTAAATTGGCTTCACATATGTATTGTTCTTTCTACCCTCCAGATGAGGATGAAGA |
| | | AGAAGGTGATTGTGAAGAAGAAGAGTTTGAGCCATCAACTCAATATGAGTATGGTAC |
| | | TGAAGATGATTACCAAGGTAAACCTTTGGAATTTGGTGCCACTTCTGCTGCTCTTCAA |
| | | CCTGAAGAAGAGCAAGAAGAAGATTGGTTAGATGATGATAGTCAACAAACTGTTGGT |
| | | CAACAAGACGGCAGTGAGGACAATCAGACAACTACTATTCAAACAATTGTTGAGGTT |
| | | CAACCTCAATTAGAGATGGAACTTACACCAGTTGTTCAGACTATTGAAGTGAATAGTT |
| | | TTAGTGGTTATTTAAAACTTACTGACAATGTATACATTAAAAATGCAGACATTGTGGAA |
| | | GAAGCTAAAAAGGTAAAACCAACAGTGGTTGTTAATGCAGCCAATGTTTACCTTAAAC |
| | | ATGGAGGAGGTGTTGCAGGAGCCTTAAATAAGGCTACTAACAATGCCATGCAAGTTG |
| | | AATCTGATGATTACATAGCTACTAATGGACCACTTAAAGTGGGTGGTAGTTGTGTTTT |
| | | AAGCGGACACAATCTTGCTAAACACTGTCTTCATGTTGTCGGCCCAAATGTTAACAAA |
| | | GGTGAAGACATTCAACTTCTTAAGAGTGCTTATGAAAATTTTAATCAGCACGAAGTTC |
| | | TACTTGCACCATTATTATCAGCTGGTATTTTTGGTGCTGACCCTATACATTCTTTAAGA |
| | | GTTTGTGTAGATACTGTTCGCACAAATGTCTACTTAGCTGTCTTTGATAAAAATCTCTA |
| | | TGACAAACTTGTTTCAAGCTTTTTGGAAATGAAGAGTGAAAAGCAAGTTGAACAAAAG |
| | | ATCGCTGAGATTCCTAAAGAGGAAGTTAAGCCATTTATAACTGAAAGTAAACCTTCAG |
| | | TTGAACAGAGAAAACAAGATGATAAGAAAATCAAAGCTTGTGTTGAAGAAGTTACAAC |
| | | AACTCTGGAAGAAACTAAGTTCCTCACAGAAAACTTGTTACTTTATATTGACATTAATG |
| | | GCAATCTTCATCCAGATTCTGCCACTCTTGTTAGTGACATTGACATCACTTTCTTAAAG |
| | | AAAGATGCTCCATATATAGTGGGTGATGTTGTTCAAGAGGGTGTTTTAACTGCTGTGG |
| | | TTATACCTACTAAAAAGGCTGGTGGCACTACTGAAATGCTAGCGAAAGCTTTGAGAAA |
| | | AGTGCCAACAGACAATTATATAACCACTTACCCGGGTCAGGGTTTAAATGGTTACACT |
| | | GTAGAGGAGGCAAAGACAGTGCTTAAAAAGTGTAAAAGTGCCTTTTACATTCTACCAT |
| | | CTATTATCTCTAATGAGAAGCAAGAAATTCTTGGAACTGTTTCTTGGAATTTGCGAGA |
| | | AATGCTTGCACATGCAGAAGAAACACGCAAATTAATGCCTGTCTGTGTGGAAACTAAA |
| | | GCCATAGTTTCAACTATACAGCGTAAATATAAGGGTATTAAAATACAAGAGGGTGTGG |
| | | TTGATTATGGTGCTAGATTTTACTTTTACACCAGTAAAACAACTGTAGCGTCACTTATC |
| | | AACACACTTAACGATCTAAATGAAACTCTTGTTACAATGCCACTTGGCTATGTAACAC |
| | | ATGGCTTAAATTTGGAAGAAGCTGCTCGGTATATGAGATCTCTCAAAGTGCCAGCTA |
| | | CAGTTTCTGTTTCTTCACCTGATGCTGTTACAGCGTATAATGGTTATCTTACTTCTTCT |
| | | TCTAAAACACCTGAAGAACATTTTATTGAAACCATCTCACTTGCTGGTTCCTATAAAGA |
| | | TTGGTCCTATTCTGGACAATCTACACAACTAGGTATAGAATTTCTTAAGAGAGGTGAT |
| | | AAAAGTGTATATTACACTAGTAATCCTACCACATTCCACCTAGATGGTGAAGTTATCA |
| | | CCTTTGACAATCTTAAGACACTTCTTTCTTTGAGAGAAGTGAGGACTATTAAGGTGTT |
| | | TACAACAGTAGACAACATTAACCTCCACACGCAAGTTGTGGACATGTCAATGACATAT |
| | | GGACAACAGTTTGGTCCAACTTATTTGGATGGAGCTGATGTTACTAAAATAAAACCTC |
| | | ATAATTCACATGAAGGTAAAACATTTTATGTTTTACCTAATGATGACACTCTACGTGTT |
| | | GAGGCTTTTGAGTACTACCACACAACTGATCCTAGTTTTCTGGGTAGGTACATGTCAG |
| | | CATTAAATCACACTAAAAAGTGGAAATACCCACAAGTTAATGGTTTAACTTCTATTAAA |
| | | TGGGCAGATAACAACTGTTATCTTGCCACTGCATTGTTAACACTCCAACAAATAGAGT |
| | | TGAAGTTTAATCCACCTGCTCTACAAGATGCTTATTACAGAGCAAGGGCTGGTGAAG |
| | | CTGCTAACTTTTGTGCACTTATCTTAGCCTACTGTAATAAGACAGTAGGTGAGTTAGG |
| | | TGATGTTAGAGAAACAATGAGTTACTTGTTTCAACATGCCAATTTAGATTCTTGCAAAA |
| | | GAGTCTTGAACGTGGTGTGTAAAACTTGTGGACAACAGCAGACAACCCTTAAGGGTG |
| | | TAGAAGCTGTTATGTACATGGGCACACTTTCTTATGAACAATTTAAGAAAGGTGTTCA |
| | | GATACCTTGTACGTGTGGTAAACAAGCTACAAAATATCTAGTACAACAGGAGTCACCT |
| | | TTTGTTATGATGTCAGCACCACCTGCTCAGTATGAACTTAAGCATGGTACATTTACTT |
| | | GTGCTAGTGAGTACACTGGTAATTACCAGTGTGGTCACTATAAACATATAACTTCTAA |
| | | AGAAACTTTGTATTGCATAGACGGTGCTTTACTTACAAAGTCCTCAGAATACAAAGGT |
| | | CCTATTACGGATGTTTTCTACAAAGAAAACAGTTACACAACAACCATAAAACCAGTTA |
| | | CTTATAAATTGGATGGTGTTGTTTGTACAGAAATTGACCCTAAGTTGGACAATTATTAT |
| | | AAGAAAGACAATTCTTATTTCACAGAGCAACCAATTGATCTTGTACCAAACCAACCAT |
| | | ATCCAAACGCAAGCTTCGATAATTTTAAGTTTGTATGTGATAATATCAAATTTGCTGAT |
| | | GATTTAAACCAGTTAACTGGTTATAAGAAACCTGCTTCAAGAGAGCTTAAAGTTACAT |
| | | TTTTCCCTGACTTAAATGGTGATGTGGTGGCTATTGATTATAAACACTACACACCCTC |
| | | TTTTAAGAAAGGAGCTAAATTGTTACATAAACCTATTGTTTGGCATGTTAACAATGCAA |
| | | CTAATAAAGCCACGTATAAACCAAATACCTGGTGTATACGTTGTCTTTGGAGCACAAA |
| | | ACCAGTTGAAACATCAAATTCGTTTGATGTACTGAAGTCAGAGGACGCGCAGGGAAT |
| | | GGATAATCTTGCCTGCGAAGATCTAAAACCAGTCTCTGAAGAAGTAGTGGAAAATCC |
| | | TACCATACAGAAAGACGTTCTTGAGTGTAATGTGAAAACTACCGAAGTTGTAGGAGA |
| | | CATTATACTTAAACCAGCAAATAATAGTTTAAAAATTACAGAAGAGGTTGGCCACACA |
| | | GATCTAATGGCTGCTTATGTAGACAATTCTAGTCTTACTATTAAGAAACCTAATGAATT |
| | | ATCTAGAGTATTAGGTTTGAAAACCCTTGCTACTCATGGTTTAGCTGCTGTTAATAGT |
| | | GTCCCTTGGGATACTATAGCTAATTATGCTAAGCCTTTTCTTAACAAAGTTGTTAGTAC |
| | | AACTACTAACATAGTTACACGGTGTTTAAACCGTGTTTGTACTAATTATATGCCTTATT |
| | | TCTTTACTTTATTGCTACAATTGTGTACTTTTACTAGAAGTACAAATTCTAGAATTAAAG |
| | | CATCTATGCCGACTACTATAGCAAAGAATACTGTTAAGAGTGTCGGTAAATTTTGTCT |
| | | AGAGGCTTCATTTAATTATTTGAAGTCACCTAATTTTTCTAAACTGATAAATATTATAAT |
| | | TTGGTTTTTACTATTAAGTGTTTGCCTAGGTTCTTTAATCTACTCAACCGCTGCTTTAG |
| | | GTGTTTTAATGTCTAATTTAGGCATGCCTTCTTACTGTACTGGTTACAGAGAAGGCTA |
| | | TTTGAACTCTACTAATGTCACTATTGCAACCTACTGTACTGGTTCTATACCTTGTAGTG |
| | | TTTGTCTTAGTGGTTTAGATTCTTTAGACACCTATCCTTCTTTAGAAACTATACAAATTA |
| | | CCATTTCATCTTTTAAATGGGATTTAACTGCTTTTGGCTTAGTTGCAGAGTGGTTTTTG |
| | | GCATATATTCTTTTCACTAGGTTTTTCTATGTACTTGGATTGGCTGCAATCATGCAATT |
| | | GTTTTTCAGCTATTTTGCAGTACATTTTATTAGTAATTCTTGGCTTATGTGGTTAATAAT |
| | | TAATCTTGTACAAATGGCCCCGATTTCAGCTATGGTTAGAATGTACATCTTCTTTGCAT |
| | | CATTTTATTATGTATGGAAAAGTTATGTGCATGTTGTAGACGGTTGTAATTCATCAACT |
| | | TGTATGATGTGTTACAAACGTAATAGAGCAACAAGAGTCGAATGTACAACTATTGTTA |
| | | ATGGTGTTAGAAGGTCCTTTTATGTCTATGCTAATGGAGGTAAAGGCTTTTGCAAACT |
| | | ACACAATTGGAATTGTGTTAATTGTGATACATTCTGTGCTGGTAGTACATTTATTAGTG |
| | | ATGAAGTTGCGAGAGACTTGTCACTACAGTTTAAAAGACCAATAAATCCTACTGACCA |
| | | GTCTTCTTACATCGTTGATAGTGTTACAGTGAAGAATGGTTCCATCCATCTTTACTTTG |
| | | ATAAAGCTGGTCAAAAGACTTATGAAAGACATTCTCTCTCTCATTTTGTTAACTTAGAC |
| | | AACCTGAGAGCTAATAACACTAAAGGTTCATTGCCTATTAATGTTATAGTTTTTGATGG |
| | | TAAATCAAAATGTGAAGAATCATCTGCAAAATCAGCGTCTGTTTACTACAGTCAGCTT |
| | | ATGTGTCAACCTATACTGTTACTAGATCAGGCATTAGTGTCTGATGTTGGTGATAGTG |
| | | CGGAAGTTGCAGTTAAAATGTTTGATGCTTACGTTAATACGTTTTCATCAACTTTTAAC |
| | | GTACCAATGGAAAAACTCAAAACACTAGTTGCAACTGCAGAAGCTGAACTTGCAAAG |
| | | AATGTGTCCTTAGACAATGTCTTATCTACTTTTATTTCAGCAGCTCGGCAAGGGTTTG |
| | | TTGATTCAGATGTAGAAACTAAAGATGTTGTTGAATGTCTTAAATTGTCACATCAATCT |
| | | GACATAGAAGTTACTGGCGATAGTTGTAATAACTATATGCTCACCTATAACAAAGTTG |
| | | AAAACATGACACCCCGTGACCTTGGTGCTTGTATTGACTGTAGTGCGCGTCATATTAA |
| | | TGCGCAGGTAGCAAAAAGTCACAACATTGCTTTGATATGGAACGTTAAAGATTTCATG |
| | | TCATTGTCTGAACAACTACGAAAACAAATACGTAGTGCTGCTAAAAAGAATAACTTAC |
| | | CTTTTAAGTTGACATGTGCAACTACTAGACAAGTTGTTAATGTTGTAACAACAAAGATA |
| | | GCACTTAAGGGTGGTAAAATTGTTAATAATTGGTTGAAGCAGTTAATTAAAGTTACAC |
| | | TTGTGTTCCTTTTTGTTGCTGCTATTTTCTATTTAATAACACCTGTTCATGTCATGTCTA |
| | | AACATACTGACTTTTCAAGTGAAATCATAGGATACAAGGCTATTGATGGTGGTGTCAC |
| | | TCGTGACATAGCATCTACAGATACTTGTTTTGCTAACAAACATGCTGATTTTGACACAT |
| | | GGTTTAGCCAGCGTGGTGGTAGTTATACTAATGACAAAGCTTGCCCATTGATTGCTG |
| | | CAGTCATAACAAGAGAAGTGGGTTTTGTCGTGCCTGGTTTGCCTGGCACGATATTAC |
| | | GCACAACTAATGGTGACTTTTTGCATTTCTTACCTAGAGTTTTTAGTGCAGTTGGTAA |
| | | CATCTGTTACACACCATCAAAACTTATAGAGTACACTGACTTTGCAACATCAGCTTGT |
| | | GTTTTGGCTGCTGAATGTACAATTTTTAAAGATGCTTCTGGTAAGCCAGTACCATATT |
| | | GTTATGATACCAATGTACTAGAAGGTTCTGTTGCTTATGAAAGTTTACGCCCTGACAC |
| | | ACGTTATGTGCTCATGGATGGCTCTATTATTCAATTTCCTAACACCTACCTTGAAGGT |
| | | TCTGTTAGAGTGGTAACAACTTTTGATTCTGAGTACTGTAGGCACGGCACTTGTGAAA |
| | | GATCAGAAGCTGGTGTTTGTGTATCTACTAGTGGTAGATGGGTACTTAACAATGATTA |
| | | TTACAGATCTTTACCAGGAGTTTTCTGTGGTGTAGATGCTGTAAATTTACTTACTAATA |
| | | TGTTTACACCACTAATTCAACCTATTGGTGCTTTGGACATATCAGCATCTATAGTAGCT |
| | | GGTGGTATTGTAGCTATCGTAGTAACATGCCTTGCCTACTATTTTATGAGGTTTAGAA |
| | | GAGCTTTTGGTGAATACAGTCATGTAGTTGCCTTTAATACTTTACTATTCCTTATGTCA |
| | | TTCACTGTACTCTGTTTAACACCAGTTTACTCATTCTTACCTGGTGTTTATTCTGTTATT |
| | | TACTTGTACTTGACATTTTATCTTACTAATGATGTTTCTTTTTTAGCACATATTCAGTGG |
| | | ATGGTTATGTTCACACCTTTAGTACCTTTCTGGATAACAATTGCTTATATCATTTGTAT |
| | | TTCCACAAAGCATTTCTATTGGTTCTTTAGTAATTACCTAAAGAGACGTGTAGTCTTTA |
| | | ATGGTGTTTCCTTTAGTACTTTTGAAGAAGCTGCGCTGTGCACCTTTTTGTTAAATAAA |
| | | GAAATGTATCTAAAGTTGCGTAGTGATGTGCTATTACCTCTTACGCAATATAATAGATA |
| | | CTTAGCTCTTTATAATAAGTACAAGTATTTTAGTGGAGCAATGGATACAACTAGCTACA |
| | | GAGAAGCTGCTTGTTGTCATCTCGCAAAGGCTCTCAATGACTTCAGTAACTCAGGTT |
| | | CTGATGTTCTTTACCAACCACCACAAACCTCTATCACCTCAGCTGTTTTGCAGAGTGG |
| | | TTTTAGAAAAATGGCATTCCCATCTGGTAAAGTTGAGGGTTGTATGGTACAAGTAACT |
| | | TGTGGTACAACTACACTTAACGGTCTTTGGCTTGATGACGTAGTTTACTGTCCAAGAC |
| | | ATGTGATCTGCACCTCTGAAGACATGCTTAACCCTAATTATGAAGATTTACTCATTCG |
| | | TAAGTCTAATCATAATTTCTTGGTACAGGCTGGTAATGTTCAACTCAGGGTTATTGGA |
| | | CATTCTATGCAAAATTGTGTACTTAAGCTTAAGGTTGATACAGCCAATCCTAAGACAC |
| | | CTAAGTATAAGTTTGTTCGCATTCAACCAGGACAGACTTTTTCAGTGTTAGCTTGTTA |
| | | CAATGGTTCACCATCTGGTGTTTACCAATGTGCTATGAGGCCCAATTTCACTATTAAG |
| | | GGTTCATTCCTTAATGGTTCATGTGGTAGTGTTGGTTTTAACATAGATTATGACTGTGT |
| | | CTCTTTTTGTTACATGCACCATATGGAATTACCAACTGGAGTTCATGCTGGCACAGAC |
| | | TTAGAAGGTAACTTTTATGGACCTTTTGTTGACAGGCAAACAGCACAAGCAGCTGGTA |
| | | CGGACACAACTATTACAGTTAATGTTTTAGCTTGGTTGTACGCTGCTGTTATAAATGG |
| | | AGACAGGTGGTTTCTCAATCGATTTACCACAACTCTTAATGACTTTAACCTTGTGGCT |
| | | ATGAAGTACAATTATGAACCTCTAACACAAGACCATGTTGACATACTAGGACCTCTTT |
| | | CTGCTCAAACTGGAATTGCCGTTTTAGATATGTGTGCTTCATTAAAAGAATTACTGCA |
| | | AAATGGTATGAATGGACGTACCATATTGGGTAGTGCTTTATTAGAAGATGAATTTACA |
| | | CCTTTTGATGTTGTTAGACAATGCTCAGGTGTTACTTTCCAAAGTGCAGTGAAAAGAA |
| | | CAATCAAGGGTACACACCACTGGTTGTTACTCACAATTTTGACTTCACTTTTAGTTTTA |
| | | GTCCAGAGTACTCAATGGTCTTTGTTCTTTTTTTTGTATGAAAATGCCTTTTTACCTTTT |
| | | GCTATGGGTATTATTGCTATGTCTGCTTTTGCAATGATGTTTGTCAAACATAAGCATG |
| | | CATTTCTCTGTTTGTTTTTGTTACCTTCTCTTGCCACTGTAGCTTATTTTAATATGGTCT |
| | | ATATGCCTGCTAGTTGGGTGATGCGTATTATGACATGGTTGGATATGGTTGATACTAG |
| | | TTTGTCTGGTTTTAAGCTAAAAGACTGTGTTATGTATGCATCAGCTGTAGTGTTACTAA |
| | | TCCTTATGACAGCAAGAACTGTGTATGATGATGGTGCTAGGAGAGTGTGGACACTTA |
| | | TGAATGTCTTGACACTCGTTTATAAAGTTTATTATGGTAATGCTTTAGATCAAGCCATT |
| | | TCCATGTGGGCTCTTATAATCTCTGTTACTTCTAACTACTCAGGTGTAGTTACAACTGT |
| | | CATGTTTTTGGCCAGAGGTATTGTTTTTATGTGTGTTGAGTATTGCCCTATTTTCTTCA |
| | | TAACTGGTAATACACTTCAGTGTATAATGCTAGTTTATTGTTTCTTAGGCTATTTTTGT |
| | | ACTTGTTACTTTGGCCTCTTTTGTTTACTCAACCGCTACTTTAGACTGACTCTTGGTGT |
| | | TTATGATTACTTAGTTTCTACACAGGAGTTTAGATATATGAATTCACAGGGACTACTCC |
| | | CACCCAAGAATAGCATAGATGCCTTCAAACTCAACATTAAATTGTTGGGTGTTGGTGG |
| | | CAAACCTTGTATCAAAGTAGCCACTGTACAGTCTAAAATGTCAGATGTAAAGTGCACA |
| | | TCAGTAGTCTTACTCTCAGTTTTGCAACAACTCAGAGTAGAATCATCATCTAAATTGTG |
| | | GGCTCAATGTGTCCAGTTACACAATGACATTCTCTTAGCTAAAGATACTACTGAAGCC |
| | | TTTGAAAAAATGGTTTCACTACTTTCTGTTTTGCTTTCCATGCAGGGTGCTGTAGACAT |
| | | AAACAAGCTTTGTGAAGAAATGCTGGACAACAGGGCAACCTTACAAGCTATAGCCTC |
| | | AGAGTTTAGTTCCCTTCCATCATATGCAGCTTTTGCTACTGCTCAAGAAGCTTATGAG |
| | | CAGGCTGTTGCTAATGGTGATTCTGAAGTTGTTCTTAAAAAGTTGAAGAAGTCTTTGA |
| | | ATGTGGCTAAATCTGAATTTGACCGTGATGCAGCCATGCAACGTAAGTTGGAAAAGA |
| | | TGGCTGATCAAGCTATGACCCAAATGTATAAACAGGCTAGATCTGAGGACAAGAGGG |
| | | CAAAAGTTACTAGTGCTATGCAGACAATGCTTTTCACTATGCTTAGAAAGTTGGATAA |
| | | TGATGCACTCAACAACATTATCAACAATGCAAGAGATGGTTGTGTTCCCTTGAACATA |
| | | ATACCTCTTACAACAGCAGCCAAACTAATGGTTGTCATACCAGACTATAACACATATA |
| | | AAAATACGTGTGATGGTACAACATTTACTTATGCATCAGCATTGTGGGAAATCCAACA |
| | | GGTTGTAGATGCAGATAGTAAAATTGTTCAACTTAGTGAAATTAGTATGGACAATTCA |
| | | CCTAATTTAGCATGGCCTCTTATTGTAACAGCTTTAAGGGCCAATTCTGCTGTCAAAT |
| | | TACAGAATAATGAGCTTAGTCCTGTTGCACTACGACAGATGTCTTGTGCTGCCGGTA |
| | | CTACACAAACTGCTTGCACTGATGACAATGCGTTAGCTTACTACAACACAACAAAGG |
| | | GAGGTAGGTTTGTACTTGCACTGTTATCCGATTTACAGGATTTGAAATGGGCTAGATT |
| | | CCCTAAGAGTGATGGAACTGGTACTATCTATACAGAACTGGAACCACCTTGTAGGTTT |
| | | GTTACAGACACACCTAAAGGTCCTAAAGTGAAGTATTTATACTTTATTAAAGGATTAAA |
| | | CAACCTAAATAGAGGTATGGTACTTGGTAGTTTAGCTGCCACAGTACGTCTACAAGCT |
| | | GGTAATGCAACAGAAGTGCCTGCCAATTCAACTGTATTATCTTTCTGTGCTTTTGCTG |
| | | TAGATGCTGCTAAAGCTTACAAAGATTATCTAGCTAGTGGGGGACAACCAATCACTAA |
| | | TTGTGTTAAGATGTTGTGTACACACACTGGTACTGGTCAGGCAATAACAGTTACACCG |
| | | GAAGCCAATATGGATCAAGAATCCTTTGGTGGTGCATCGTGTTGTCTGTACTGCCGT |
| | | TGCCACATAGATCATCCAAATCCTAAAGGATTTTGTGACTTAAAAGGTAAGTATGTAC |
| | | AAATACCTACAACTTGTGCTAATGACCCTGTGGGTTTTACACTTAAAAACACAGTCTG |
| | | TACCGTCTGCGGTATGTGGAAAGGTTATGGCTGTAGTTGTGATCAACTCCGCGAACC |
| | | CATGCTTCAGTCAGCTGATGCACAATCGTTTTTAAACGGGTTTGCGGTGTAAGTGCA |
| | | GCCCGTCTTACACCGTGCGGCACAGGCACTAGTACTGATGTCGTATACAGGGCTTTT |
| | | GACATCTACAATGATAAAGTAGCTGGTTTTGCTAAATTCCTAAAAACTAATTGTTGTCG |
| | | CTTCCAAGAAAAGGACGAAGATGACAATTTAATTGATTCTTACTTTGTAGTTAAGAGA |
| | | CACACTTTCTCTAACTACCAACATGAAGAAACAATTTATAATTTACTTAAGGATTGTCC |
| | | AGCTGTTGCTAAACATGACTTCTTTAAGTTTAGAATAGACGGTGACATGGTACCACAT |
| | | ATATCACGTCAACGTCTTACTAAATACACAATGGCAGACCTCGTCTATGCTTTAAGGC |
| | | ATTTTGATGAAGGTAATTGTGACACATTAAAAGAAATACTTGTCACATACAATTGTTGT |
| | | GATGATGATTATTTCAATAAAAAGGACTGGTATGATTTTGTAGAAAACCCAGATATATT |
| | | ACGCGTATACGCCAACTTAGGTGAACGTGTACGCCAAGCTTTGTTAAAAACAGTACA |
| | | ATTCTGTGATGCCATGCGAAATGCTGGTATTGTTGGTGTACTGACATTAGATAATCAA |
| | | GATCTCAATGGTAACTGGTATGATTTCGGTGATTTCATACAAACCACGCCAGGTAGTG |
| | | GAGTTCCTGTTGTAGATTCTTATTATTCATTGTTAATGCCTATATTAACCTTGACCAGG |
| | | GCTTTAACTGCAGAGTCACATGTTGACACTGACTTAACAAAGCCTTACATTAAGTGGG |
| | | ATTTGTTAAAATATGACTTCACGGAAGAGAGGTTAAAACTCTTTGACCGTTATTTTAAA |
| | | TATTGGGATCAGACATACCACCCAAATTGTGTTAACTGTTTGGATGACAGATGCATTC |
| | | TGCATTGTGCAAACTTTAATGTTTTATTCTCTACAGTGTTCCCACCTACAAGTTTTGGA |
| | | CCACTAGTGAGAAAAATATTTGTTGATGGTGTTCCATTTGTAGTTTCAACTGGATACC |
| | | ACTTCAGAGAGCTAGGTGTTGTACATAATCAGGATGTAAACTTACATAGCTCTAGACT |
| | | TAGTTTTAAGGAATTACTTGTGTATGCTGCTGACCCTGCTATGCACGCTGCTTCTGGT |
| | | AATCTATTACTAGATAAACGCACTACGTGCTTTTCAGTAGCTGCACTTACTAACAATGT |
| | | TGCTTTTCAAACTGTCAAACCCGGTAATTTTAACAAAGACTTCTATGACTTTGCTGTGT |
| | | CTAAGGGTTTCTTTAAGGAAGGAAGTTCTGTTGAATTAAAACACTTCTTCTTTGCTCA |
| | | GGATGGTAATGCTGCTATCAGCGATTATGACTACTATCGTTATAATCTACCAACAATG |
| | | TGTGATATCAGACAACTACTATTTGTAGTTGAAGTTGTTGATAAGTACTTTGATTGTTA |
| | | CGATGGTGGCTGTATTAATGCTAACCAAGTCATCGTCAACAACCTAGACAAATCAGCT |
| | | GGTTTTCCATTTAATAAATGGGGTAAGGCTAGACTTTATTATGATTCAATGAGTTATGA |
| | | GGATCAAGATGCACTTTTCGCATATACAAAACGTAATGTCATCCCTACTATAACTCAA |
| | | ATGAATCTTAAGTATGCCATTAGTGCAAAGAATAGAGCTCGCACCGTAGCTGGTGTC |
| | | TCTATCTGTAGTACTATGACCAATAGACAGTTTCATCAAAAATTATTGAAATCAATAGC |
| | | CGCCACTAGAGGAGCTACTGTAGTAATTGGAACAAGCAAATTCTATGGTGGTTGGCA |
| | | CAACATGTTAAAAACTGTTTATAGTGATGTAGAAAACCCTCACCTTATGGGTTGGGAT |
| | | TATCCTAAATGTGATAGAGCCATGCCTAACATGCTTAGAATTATGGCCTCACTTGTTC |
| | | TTGCTCGCAAACATACAACGTGTTGTAGCTTGTCACACCGTTTCTATAGATTAGCTAA |
| | | TGAGTGTGCTCAAGTATTGAGTGAAATGGTCATGTGTGGCGGTTCACTATATGTTAAA |
| | | CCAGGTGGAACCTCATCAGGAGATGCCACAACTGCTTATGCTAATAGTGTTTTTAACA |
| | | TTTGTCAAGCTGTCACGGCCAATGTTAATGCACTTTTATCTACTGATGGTAACAAAATT |
| | | GCCGATAAGTATGTCCGCAATTTACAACACAGACTTTATGAGTGTCTCTATAGAAATA |
| | | GAGATGTTGACACAGACTTTGTGAATGAGTTTTACGCATATTTGCGTAAACATTTCTC |
| | | AATGATGATACTCTCTGACGATGCTGTTGTGTGTTTCAATAGCACTTATGCATCTCAA |
| | | GGTCTAGTGGCTAGCATAAAGAACTTTAAGTCAGTTCTTTATTATCAAAACAATGTTTT |
| | | TATGTCTGAAGCAAAATGTTGGACTGAGACTGACCTTACTAAAGGACCTCATGAATTT |
| | | TGCTCTCAACATACAATGCTAGTTAAACAGGGTGATGATTATGTGTACCTTCCTTACC |
| | | CAGATCCATCAAGAATCCTAGGGGCCGGCTGTTTTGTAGATGATATCGTAAAAACAG |
| | | ATGGTACACTTATGATTGAACGGTTCGTGTCTTTAGCTATAGATGCTTACCCACTTAC |
| | | TAAACATCCTAATCAGGAGTATGCTGATGTCTTTCATTTGTACTTACAATACATAAGAA |
| | | AGCTACATGATGAGTTAACAGGACACATGTTAGACATGTATTCTGTTATGCTTACTAA |
| | | TGATAACACTTCAAGGTATTGGGAACCTGAGTTTTATGAGGCTATGTACACACCGCAT |
| | | ACAGTCTTACAGGCTGTTGGGGCTTGTGTTCTTTGCAATTCACAGACTTCATTAAGAT |
| | | GTGGTGCTTGCATACGTAGACCATTCTTATGTTGTAAATGCTGTTACGACCATGTCAT |
| | | ATCAACATCACATAAATTAGTCTTGTCTGTTAATCCGTATGTTTGCAATGCTCCAGGTT |
| | | GTGATGTCACAGATGTGACTCAACTTTACTTAGGAGGTATGAGCTATTATTGTAAATC |
| | | ACATAAACCACCCATTAGTTTTCCATTGTGTGCTAATGGACAAGTTTTTGGTTTATATA |
| | | AAAATACATGTGTTGGTAGCGATAATGTTACTGACTTTAATGCAATTGCAACATGTGA |
| | | CTGGACAAATGCTGGTGATTACATTTTAGCTAACACCTGTACTGAAAGACTCAAGCTT |
| | | TTTGCAGCAGAAACGCTCAAAGCTACTGAGGAGACATTTAAACTGTCTTATGGTATTG |
| | | CTACTGTACGTGAAGTGCTGTCTGACAGAGAATTACATCTTTCATGGGAAGTTGGTAA |
| | | ACCTAGACCACCACTTAACCGAAATTATGTCTTTACTGGTTATCGTGTAACTAAAAAC |
| | | AGTAAAGTACAAATAGGAGAGTACACCTTTGAAAAAGGTGACTATGGTGATGCTGTT |
| | | GTTTACCGAGGTACAACAACTTACAAATTAAATGTTGGTGATTATTTTGTGCTGACATC |
| | | ACATACAGTAATGCCATTAAGTGCACCTACACTAGTGCCACAAGAGCACTATGTTAGA |
| | | ATTACTGGCTTATACCCAACACTCAATATCTCAGATGAGTTTTCTAGCAATGTTGCAAA |
| | | TTATCAAAAGGTTGGTATGCAAAAGTATTCTACACTCCAGGGACCACCTGGTACTGGT |
| | | AAGAGTCATTTTGCTATTGGCCTAGCTCTCTACTACCCTTCTGCTCGCATAGTGTATA |
| | | CAGCTTGCTCTCATGCCGCTGTTGATGCACTATGTGAGAAGGCATTAAAATATTTGCC |
| | | TATAGATAAATGTAGTAGAATTATACCTGCACGTGCTCGTGTAGAGTGTTTTGATAAA |
| | | TTCAAAGTGAATTCAACATTAGAACAGTATGTCTTTTGTACTGTAAATGCATTGCCTGA |
| | | GACGACAGCAGATATAGTTGTCTTTGATGAAATTTCAATGGCCACAAATTATGATTTG |
| | | AGTGTTGTCAATGCCAGATTACGTGCTAAGCACTATGTGTACATTGGCGACCCTGCT |
| | | CAATTACCTGCACCACGCACATTGCTAACTAAGGGCACACTAGAACCAGAATATTTCA |
| | | ATTCAGTGTGTAGACTTATGAAAACTATAGGTCCAGACATGTTCCTCGGAACTTGTCG |
| | | GCGTTGTCCTGCTGAAATTGTTGACACTGTGAGTGCTTTGGTTTATGATAATAAGCTT |
| | | AAAGCACATAAAGACAAATCAGCTCAATGCTTTAAAATGTTTTATAAGGGTGTTATCAC |
| | | GCATGATGTTTCATCTGCAATTAACAGGCCACAAATAGGCGTGGTAAGAGAATTCCTT |
| | | ACACGTAACCCTGCTTGGAGAAAAGCTGTCTTTATTTCACCTTATAATTCACAGAATG |
| | | CTGTAGCCTCAAAGATTTTGGGACTACCAACTCAAACTGTTGATTCATCACAGGGCTC |
| | | AGAATATGACTATGTCATATTCACTCAAACCACTGAAACAGCTCACTCTTGTAATGTAA |
| | | ACAGATTTAATGTTGCTATTACCAGAGCAAAAGTAGGCATACTTTGCATAATGTCTGA |
| | | TAGAGACCTTTATGACAAGTTGCAATTTACAAGTCTTGAAATTCCACGTAGGAATGTG |
| | | GCAACTTTACAAGCTGAAAATGTAACAGGACTCTTTAAAGATTGTAGTAAGGTAATCA |
| | | CTGGGTTACATCCTACACAGGCACCTACACACCTCAGTGTTGACACTAAATTCAAAAC |
| | | TGAAGGTTTATGTGTTGACATACCTGGCATACCTAAGGACATGACCTATAGAAGACTC |
| | | ATCTCTATGATGGGTTTTAAAATGAATTATCAAGTTAATGGTTACCCTAACATGTTTAT |
| | | CACCCGCGAAGAAGCTATAAGACATGTACGTGCATGGATTGGCTTCGATGTCGAGG |
| | | GGTGTCATGCTACTAGAGAAGCTGTTGGTACCAATTTACCTTTACAGCTAGGTTTTTC |
| | | TACAGGTGTTAACCTAGTTGCTGTACCTACAGGTTATGTTGATACACCTAATAATACA |
| | | GATTTTTCCAGAGTTAGTGCTAAACCACCGCCTGGAGATCAATTTAAACACCTCATAC |
| | | CACTTATGTACAAAGGACTTCCTTGGAATGTAGTGCGTATAAAGATTGTACAAATGTT |
| | | AAGTGACACACTTAAAAATCTCTCTGACAGAGTCGTATTTGTCTTATGGGCACATGGC |
| | | TTTGAGTTGACATCTATGAAGTATTTTGTGAAAATAGGACCTGAGCGCACCTGTTGTC |
| | | TATGTGATAGACGTGCCACATGCTTTTCCACTGCTTCAGACACTTATGCCTGTTGGCA |
| | | TCATTCTATTGGATTTGATTACGTCTATAATCCGTTTATGATTGATGTTCAACAATGGG |
| | | GTTTTACAGGTAACCTACAAAGCAACCATGATCTGTATTGTCAAGTCCATGGTAATGC |
| | | ACATGTAGCTAGTTGTGATGCAATCATGACTAGGTGTCTAGCTGTCCACGAGTGCTTT |
| | | GTTAAGCGTGTTGACTGGACTATTGAATATCCTATAATTGGTGATGAACTGAAGATTA |
| | | ATGCGGCTTGTAGAAAGGTTCAACACATGGTTGTTAAAGCTGCATTATTAGCAGACAA |
| | | ATTCCCAGTTCTTCACGACATTGGTAACCCTAAAGCTATTAAGTGTGTACCTCAAGCT |
| | | GATGTAGAATGGAAGTTCTATGATGCACAGCCTTGTAGTGACAAAGCTTATAAAATAG |
| | | AAGAATTATTCTATTCTTATGCCACACATTCTGACAAATTCACAGATGGTGTATGCCTA |
| | | TTTTGGAATTGCAATGTCGATAGATATCCTGCTAATTCCATTGTTTGTAGATTTGACAC |
| | | TAGAGTGCTATCTAACCTTAACTTGCCTGGTTGTGATGGTGGCAGTTTGTATGTAAAT |
| | | AAACATGCATTCCACACACCAGCTTTTGATAAAAGTGCTTTTGTTAATTTAAAACAATT |
| | | ACCATTTTTCTATTACTCTGACAGTCCATGTGAGTCTCATGGAAAACAAGTAGTGTCA |
| | | GATATAGATTATGTACCACTAAAGTCTGCTACGTGTATAACACGTTGCAATTTAGGTG |
| | | GTGCTGTCTGTAGACATCATGCTAATGAGTACAGATTGTATCTCGATGCTTATAACAT |
| | | GATGATCTCAGCTGGCTTTAGCTTGTGGGTTTACAAACAATTTGATACTTATAACCTC |
| | | TGGAACACTTTTACAAGACTTCAGAGTTTAGAAAATGTGGCTTTTAATGTTGTAAATAA |
| | | GGGACACTTTGATGGACAACAGGGTGAAGTACCAGTTTCTATCATTAATAACACTGTT |
| | | TACACAAAAGTTGATGGTGTTGATGTAGAATTGTTTGAAAATAAAACAACATTACCTGT |
| | | TAATGTAGCATTTGAGCTTTGGGCTAAGCGCAACATTAAACCAGTACCAGAGGTGAA |
| | | AATACTCAATAATTTGGGTGTGGACATTGCTGCTAATACTGTGATCTGGGACTACAAA |
| | | AGAGATGCTCCAGCACATATATCTACTATTGGTGTTTGTTCTATGACTGACATAGCCA |
| | | AGAAACCAACTGAAACGATTTGTGCACCACTCACTGTCTTTTTTGATGGTAGAGTTGA |
| | | TGGTCAAGTAGACTTATTTAGAAATGCCCGTAATGGTGTTCTTATTACAGAAGGTAGT |
| | | GTTAAAGGTTTACAACCATCTGTAGGTCCCAAACAAGCTAGTCTTAATGGAGTCACAT |
| | | TAATTGGAGAAGCCGTAAAAACACAGTTCAATTATTATAAGAAAGTTGATGGTGTTGT |
| | | CCAACAATTACCTGAAACTTACTTTACTCAGAGTAGAAATTTACAAGAATTTAAACCCA |
| | | GGAGTCAAATGGAAATTGATTTCTTAGAATTAGCTATGGATGAATTCATTGAACGGTA |
| | | TAAATTAGAAGGCTATGCCTTCGAACATATCGTTTATGGAGATTTTAGTCATAGTCAG |
| | | TTAGGTGGTTTACATCTACTGATTGGACTAGCTAAACGTTTTAAGGAATCACCTTTTG |
| | | AATTAGAAGATTTTATTCCTATGGACAGTACAGTTAAAAACTATTTCATAACAGATGCG |
| | | CAAACAGGTTCATCTAAGTGTGTGTGTTCTGTTATTGATTTATTACTTGATGATTTTGT |
| | | TGAAATAATAAAATCCCAAGATTTATCTGTAGTTTCTAAGGTTGTCAAAGTGACTATTG |
| | | ACTATACAGAAATTTCATTTATGCTTTGGTGTAAAGATGGCCATGTAGAAACATTTTAC |
| | | CCAAAATTACAATCTAGTCAAGCGTGGCAACCGGGTGTTGCTATGCCTAATCTTTACA |
| | | AAATGCAAAGAATGCTATTAGAAAAGTGTGACCTTCAAAATTATGGTGATAGTGCAAC |
| | | ATTACCTAAAGGCATAATGATGAATGTCGCAAAATATACTCAACTGTGTCAATATTTAA |
| | | ACACATTAACATTAGCTGTACCCTATAATATGAGAGTTATACATTTTGGTGCTGGTTCT |
| | | GATAAAGGAGTTGCACCAGGTACAGCTGTTTTAAGACAGTGGTTGCCTACGGGTACG |
| | | CTGCTTGTCGATTCAGATCTTAATGACTTTGTCTCTGATGCAGATTCAACTTTGATTG |
| | | GTGATTGTGCAACTGTACATACAGCTAATAAATGGGATCTCATTATTAGTGATATGTA |
| | | CGACCCTAAGACTAAAAATGTTACAAAAGAAAATGACTCTAAAGAGGGTTTTTTCACT |
| | | TACATTTGTGGGTTTATACAACAAAAGCTAGCTCTTGGAGGTTCCGTGGCTATAAAGA |
| | | TAACAGAACATTCTTGGAATGCTGATCTTTATAAGCTCATGGGACACTTCGCATGGTG |
| | | GACAGCCTTTGTTACTAATGTGAATGCGTCATCATCTGAAGCATTTTTAATTGGATGT |
| | | AATTATCTTGGCAAACCACGCGAACAAATAGATGGTTATGTCATGCATGCAAATTACA |
| | | TATTTTGGAGGAATACAAATCCAATTCAGTTGTCTTCCTATTCTTTATTTGACATGAGT |
| | | AAATTTCCCCTTAAATTAAGGGGTACTGCTGTTATGTCTTTAAAAGAAGGTCAAATCAA |
| | | TGATATGATTTTATCTCTTCTTAGTAAAGGTAGACTTATAATTAGAGAAAACAACAGAG |
| | | TTGTTATTTCTAGTGATGTTCTTGTTAACAACTAAACGAACAATGTTTGTTTTTCTTGTT |
| | | TTATTGCCACTAGTCTCTAGTCAGTGTGTTAATCTTACAACCAGAACTCAATTACCCC |
| | | CTGCATACACTAATTCTTTCACACGTGGTGTTTATTACCCTGACAAAGTTTTCAGATCC |
| | | TCAGTTTTACATTCAACTCAGGACTTGTTCTTACCTTTCTTTTCCAATGTTACTTGGTT |
| | | CCATGCTATACATGTCTCTGGGACCAATGGTACTAAGAGGTTTGATAACCCTGTCCTA |
| | | CCATTTAATGATGGTGTTTATTTTGCTTCCACTGAGAAGTCTAACATAATAAGAGGCT |
| | | GGATTTTTGGTACTACTTTAGATTCGAAGACCCAGTCCCTACTTATTGTTAATAACGCT |
| | | ACTAATGTTGTTATTAAAGTCTGTGAATTTCAATTTTGTAATGATCCATTTTTGGGTGTT |
| | | TATTACCACAAAAACAACAAAAGTTGGATGGAAAGTGAGTTCAGAGTTTATTCTAGTG |
| | | CGAATAATTGCACTTTTGAATATGTCTCTCAGCCTTTTCTTATGGACCTTGAAGGAAAA |
| | | CAGGGTAATTTCAAAAATCTTAGGGAATTTGTGTTTAAGAATATTGATGGTTATTTTAA |
| | | AATATATTCTAAGCACACGCCTATTAATTTAGTGCGTGATCTCCCTCAGGGTTTTTCG |
| | | GCTTTAGAACCATTGGTAGATTTGCCAATAGGTATTAACATCACTAGGTTTCAAACTTT |
| | | ACTTGCTTTACATAGAAGTTATTTGACTCCTGGTGATTCTTCTTCAGGTTGGACAGCT |
| | | GGTGCTGCAGCTTATTATGTGGGTTATCTTCAACCTAGGACTTTTCTATTAAAATATAA |
| | | TGAAAATGGAACCATTACAGATGCTGTAGACTGTGCACTTGACCCTCTCTCAGAAACA |
| | | AAGTGTACGTTGAAATCCTTCACTGTAGAAAAAGGAATCTATCAAACTTCTAACTTTAG |
| | | AGTCCAACCAACAGAATCTATTGTTAGATTTCCTAATATTACAAACTTGTGCCCTTTTG |
| | | GTGAAGTTTTTAACGCCACCAGATTTGCATCTGTTTATGCTTGGAACAGGAAGAGAAT |
| | | CAGCAACTGTGTTGCTGATTATTCTGTCCTATATAATTCCGCATCATTTTCCACTTTTA |
| | | AGTGTTATGGAGTGTCTCCTACTAAATTAAATGATCTCTGCTTTACTAATGTCTATGCA |
| | | GATTCATTTGTAATTAGAGGTGATGAAGTCAGACAAATCGCTCCAGGGCAAACTGGA |
| | | AAGATTGCTGATTATAATTATAAATTACCAGATGATTTTACAGGCTGCGTTATAGCTTG |
| | | GAATTCTAACAATCTTGATTCTAAGGTTGGTGGTAATTATAATTACCTGTATAGATTGT |
| | | TTAGGAAGTCTAATCTCAAACCTTTTGAGAGAGATATTTCAACTGAAATCTATCAGGC |
| | | CGGTAGCACACCTTGTAATGGTGTTGAAGGTTTTAATTGTTACTTTCCTTTACAATCAT |
| | | ATGGTTTCCAACCCACTAATGGTGTTGGTTACCAACCATACAGAGTAGTAGTACTTTC |
| | | TTTTGAACTTCTACATGCACCAGCAACTGTTTGTGGACCTAAAAAGTCTACTAATTTG |
| | | GTTAAAAACAAATGTGTCAATTTCAACTTCAATGGTTTAACAGGCACAGGTGTTCTTA |
| | | CTGAGTCTAACAAAAAGTTTCTGCCTTTCCAACAATTTGGCAGAGACATTGCTGACAC |
| | | TACTGATGCTGTCCGTGATCCACAGACACTTGAGATTCTTGACATTACACCATGTTCT |
| | | TTTGGTGGTGTCAGTGTTATAACACCAGGAACAAATACTTCTAACCAGGTTGCTGTTC |
| | | TTTATCAGGATGTTAACTGCACAGAAGTCCCTGTTGCTATTCATGCAGATCAACTTAC |
| | | TCCTACTTGGCGTGTTTATTCTACAGGTTCTAATGTTTTTCAAACACGTGCAGGCTGT |
| | | TTAATAGGGGCTGAACATGTCAACAACTCATATGAGTGTGACATACCCATTGGTGCA |
| | | GGTATATGCGCTAGTTATCAGACTCAGACTAATTCTCCTCGGCGGGCACGTAGTGTA |
| | | GCTAGTCAATCCATCATTGCCTACACTATGTCACTTGGTGCAGAAAATTCAGTTGCTT |
| | | ACTCTAATAACTCTATTGCCATACCCACAAATTTTACTATTAGTGTTACCACAGAAATT |
| | | CTACCAGTGTCTATGACCAAGACATCAGTAGATTGTACAATGTACATTTGTGGTGATT |
| | | CAACTGAATGCAGCAATCTTTTGTTGCAATATGGCAGTTTTTGTACACAATTAAACCG |
| | | TGCTTTAACTGGAATAGCTGTTGAACAAGACAAAAACACCCAAGAAGTTTTTGCACAA |
| | | GTCAAACAAATTTACAAAACACCACCAATTAAAGATTTTGGTGGTTTTAATTTTTCACA |
| | | AATATTACCAGATCCATCAAAACCAAGCAAGAGGTCATTTATTGAAGATCTACTTTTCA |
| | | ACAAAGTGACACTTGCAGATGCTGGCTTCATCAAACAATATGGTGATTGCCTTGGTG |
| | | ATATTGCTGCTAGAGACCTCATTTGTGCACAAAAGTTTAACGGCCTTACTGTTTTGCC |
| | | ACCTTTGCTCACAGATGAAATGATTGCTCAATACACTTCTGCACTGTTAGCGGGTACA |
| | | ATCACTTCTGGTTGGACCTTTGGTGCAGGTGCTGCATTACAAATACCATTTGCTATGC |
| | | AAATGGCTTATAGGTTTAATGGTATTGGAGTTACACAGAATGTTCTCTATGAGAACCA |
| | | AAAATTGATTGCCAACCAATTTAATAGTGCTATTGGCAAAATTCAAGACTCACTTTCTT |
| | | CCACAGCAAGTGCACTTGGAAAACTTCAAGATGTGGTCAACCAAAATGCACAAGCTT |
| | | TAAACACGCTTGTTAAACAACTTAGCTCCAATTTTGGTGCAATTTCAAGTGTTTTAAAT |
| | | GATATCCTTTCACGTCTTGACAAAGTTGAGGCTGAAGTGCAAATTGATAGGTTGATCA |
| | | CAGGCAGACTTCAAAGTTTGCAGACATATGTGACTCAACAATTAATTAGAGCTGCAGA |
| | | AATCAGAGCTTCTGCTAATCTTGCTGCTACTAAAATGTCAGAGTGTGTACTTGGACAA |
| | | TCAAAAAGAGTTGATTTTTGTGGAAAGGGCTATCATCTTATGTCCTTCCCTCAGTCAG |
| | | CACCTCATGGTGTAGTCTTCTTGCATGTGACTTATGTCCCTGCACAAGAAAAGAACTT |
| | | CACAACTGCTCCTGCCATTTGTCATGATGGAAAAGCACACTTTCCTCGTGAAGGTGT |
| | | CTTTGTTTCAAATGGCACACACTGGTTTGTAACACAAAGGAATTTTTATGAACCACAA |
| | | ATCATTACTACAGACAACACATTTGTGTCTGGTAACTGTGATGTTGTAATAGGAATTG |
| | | TCAACAACACAGTTTATGATCCTTTGCAACCTGAATTAGACTCATTCAAGGAGGAGTT |
| | | AGATAAATATTTTAAGAATCATACATCACCAGATGTTGATTTAGGTGACATCTCTGGCA |
| | | TTAATGCTTCAGTTGTAAACATTCAAAAAGAAATTGACCGCCTCAATGAGGTTGCCAA |
| | | GAATTTAAATGAATCTCTCATCGATCTCCAAGAACTTGGAAAGTATGAGCAGTATATA |
| | | AAATGGCCATGGTACATTTGGCTAGGTTTTATAGCTGGCTTGATTGCCATAGTAATGG |
| | | TGACAATTATGCTTTGCTGTATGACCAGTTGCTGTAGTTGTCTCAAGGGCTGTTGTTC |
| | | TTGTGGATCCTGCTGCAAATTTGATGAAGACGACTCTGAGCCAGTGCTCAAAGGAGT |
| | | CAAATTACATTACACATAAACGAACTTATGGATTTGTTTATGAGAATCTTCACAATTGG |
| | | AACTGTAACTTTGAAGCAAGGTGAAATCAAGGATGCTACTCCTTCAGATTTTGTTCGC |
| | | GCTACTGCAACGATACCGATACAAGCCTCACTCCCTTTCGGATGGCTTATTGTTGGC |
| | | GTTGCACTTCTTGCTGTTTTTCAGAGCGCTTCCAAAATCATAACCCTCAAAAAGAGAT |
| | | GGCAACTAGCACTCTCCAAGGGTGTTCACTTTGTTTGCAACTTGCTGTTGTTGTTTGT |
| | | AACAGTTTACTCACACCTTTTGCTCGTTGCTGCTGGCCTTGAAGCCCCTTTTCTCTAT |
| | | CTTTATGCTTTAGTCTACTTCTTGCAGAGTATAAACTTTGTAAGAATAATAATGAGGCT |
| | | TTGGCTTTGCTGGAAATGCCGTTCCAAAAACCCATTACTTTATGATGCCAACTATTTT |
| | | CTTTGCTGGCATACTAATTGTTACGACTATTGTATACCTTACAATAGTGTAACTTCTTC |
| | | AATTGTCATTACTTCAGGTGATGGCACAACAAGTCCTATTTCTGAACATGACTACCAG |
| | | ATTGGTGGTTATACTGAAAAATGGGAATCTGGAGTAAAAGACTGTGTTGTATTACACA |
| | | GTTACTTCACTTCAGACTATTACCAGCTGTACTCAACTCAATTGAGTACAGACACTGG |
| | | TGTTGAACATGTTACCTTCTTCATCTACAATAAAATTGTTGATGAGCCTGAAGAACATG |
| | | TCCAAATTCACACAATCGACGGTTCATCCGGAGTTGTTAATCCAGTAATGGAACCAAT |
| | | TTATGATGAACCGACGACGACTACTAGCGTGCCTTTGTAAGCACAAGCTGATGAGTA |
| | | CGAACTTATGTACTCATTCGTTTCGGAAGAGACAGGTACGTTAATAGTTAATAGCGTA |
| | | CTTCTTTTTCTTGCTTTCGTGGTATTCTTGCTAGTTACACTAGCCATCCTTACTGCGCT |
| | | TCGATTGTGTGCGTACTGCTGCAATATTGTTAACGTGAGTCTTGTAAAACCTTCTTTTT |
| | | ACGTTTACTCTCGTGTTAAAAATCTGAATTCTTCTAGAGTTCCTGATCTTCTGGTCTAA |
| | | ACGAACTAAATATTATATTAGTTTTTCTGTTTGGAACTTTAATTTTAGCCATGGCAGAT |
| | | TCCAACGGTACTATTACCGTTGAAGAGCTTAAAAAGCTCCTTGAACAATGGAACCTAG |
| | | TAATAGGTTTCCTATTCCTTACATGGATTTGTCTTCTACAATTTGCCTATGCCAACAGG |
| | | AATAGGTTTTTGTATATAATTAAGTTAATTTTCCTCTGGCTGTTATGGCCAGTAACTTT |
| | | AGCTTGTTTTGTGCTTGCTGCTGTTTACAGAATAAATTGGATCACCGGTGGAATTGCT |
| | | ATCGCAATGGCTTGTCTTGTAGGCTTGATGTGGCTCAGCTACTTCATTGCTTCTTTCA |
| | | GACTGTTTGCGCGTACGCGTTCCATGTGGTCATTCAATCCAGAAACTAACATTCTTCT |
| | | CAACGTGCCACTCCATGGCACTATTCTGACCAGACCGCTTCTAGAAAGTGAACTCGT |
| | | AATCGGAGCTGTGATCCTTCGTGGACATCTTCGTATTGCTGGACACCATCTAGGACG |
| | | CTGTGACATCAAGGACCTGCCTAAAGAAATCACTGTTGCTACATCACGAACGCTTTCT |
| | | TATTACAAATTGGGAGCTTCGCAGCGTGTAGCAGGTGACTCAGGTTTTGCTGCATAC |
| | | AGTCGCTACAGGATTGGCAACTATAAATTAAACACAGACCATTCCAGTAGCAGTGAC |
| | | AATATTGCTTTGCTTGTACAGTAAGTGACAACAGATGTTTCATCTCGTTGACTTTCAG |
| | | GTTACTATAGCAGAGATATTACTAATTATTATGAGGACTTTTAAAGTTTCCATTTGGAA |
| | | TCTTGATTACATCATAAACCTCATAATTAAAAATTTATCTAAGTCACTAACTGAGAATAA |
| | | ATATTCTCAATTAGATGAAGAGCAACCAATGGAGATTGATTAAACGAACATGAAAATT |
| | | ATTCTTTTCTTGGCACTGATAACACTCGCTACTTGTGAGCTTTATCACTACCAAGAGT |
| | | GTGTTAGAGGTACAACAGTACTTTTAAAAGAACCTTGCTCTTCTGGAACATACGAGGG |
| | | CAATTCACCATTTCATCCTCTAGCTGATAACAAATTTGCACTGACTTGCTTTAGCACTC |
| | | AATTTGCTTTTGCTTGTCCTGACGGCGTAAAACACGTCTATCAGTTACGTGCCAGATC |
| | | AGTTTCACCTAAACTGTTCATCAGACAAGAGGAAGTTCAAGAACTTTACTCTCCAATT |
| | | TTTCTTATTGTTGCGGCAATAGTGTTTATAACACTTTGCTTCACACTCAAAAGAAAGAC |
| | | AGAATGATTGAACTTTCATTAATTGACTTCTATTTGTGCTTTTTAGCCTTTCTGCTATTC |
| | | CTTGTTTTAATTATGCTTATTATCTTTTGGTTCTCACTTGAACTGCAAGATCATAATGAA |
| | | ACTTGTCACGCCTAAACGAACATGAAATTTCTTGTTTTCTTAGGAATCATCACAACTGT |
| | | AGCTGCATTTCACCAAGAATGTAGTTTACAGTCATGTACTCAACATCAACCATATGTA |
| | | GTTGATGACCCGTGTCCTATTCACTTCTATTCTAAATGGTATATTAGAGTAGGAGCTA |
| | | GAAAATCAGCACCTTTAATTGAATTGTGCGTGGATGAGGCTGGTTCTAAATCACCCAT |
| | | TCAGTACATCGATATCGGTAATTATACAGTTTCCTGTTTACCTTTTACAATTAATTGCC |
| | | AGGAACCTAAATTGGGTAGTCTTGTAGTGCGTTGTTCGTTCTATGAAGACTTTTTAGA |
| | | GTATCATGACGTTCGTGTTGTTTTAGATTTCATCTAAACGAACAAACTAAAATGTCTGA |
| | | TAATGGACCCCAAAATCAGCGAAATGCACCCCGCATTACGTTTGGTGGACCCTCAGA |
| | | TTCAACTGGCAGTAACCAGAATGGAGAACGCAGTGGGGCGCGATCAAAACAACGTC |
| | | GGCCCCAAGGTTTACCCAATAATACTGCGTCTTGGTTCACCGCTCTCACTCAACATG |
| | | GCAAGGAAGACCTTAAATTCCCTCGAGGACAAGGCGTTCCAATTAACACCAATAGCA |
| | | GTCCAGATGACCAAATTGGCTACTACCGAAGAGCTACCAGACGAATTCGTGGTGGTG |
| | | ACGGTAAAATGAAAGATCTCAGTCCAAGATGGTATTTCTACTACCTAGGAACTGGGC |
| | | CAGAAGCTGGACTTCCCTATGGTGCTAACAAAGACGGCATCATATGGGTTGCAACTG |
| | | AGGGAGCCTTGAATACACCAAAAGATCACATTGGCACCCGCAATCCTGCTAACAATG |
| | | CTGCAATCGTGCTACAACTTCCTCAAGGAACAACATTGCCAAAAGGCTTCTACGCAG |
| | | AAGGGAGCAGAGGCGGCAGTCAAGCCTCTTCTCGTTCCTCATCACGTAGTCGCAAC |
| | | AGTTCAAGAAATTCAACTCCAGGCAGCAGTAGGGGAACTTCTCCTGCTAGAATGGCT |
| | | GGCAATGGCGGTGATGCTGCTCTTGCTTTGCTGCTGCTTGACAGATTGAACCAGCTT |
| | | GAGAGCAAAATGTCTGGTAAAGGCCAACAACAACAAGGCCAAACTGTCACTAAGAAA |
| | | TCTGCTGCTGAGGCTTCTAAGAAGCCTCGGCAAAAACGTACTGCCACTAAAGCATAC |
| | | AATGTAACACAAGCTTTCGGCAGACGTGGTCCAGAACAAACCCAAGGAAATTTTGGG |
| | | GACCAGGAACTAATCAGACAAGGAACTGATTACAAACATTGGCCGCAAATTGCACAA |
| | | TTTGCCCCCAGCGCTTCAGCGTTCTTCGGAATGTCGCGCATTGGCATGGAAGTCACA |
| | | CCTTCGGGAACGTGGTTGACCTACACAGGTGCCATCAAATTGGATGACAAAGATCCA |
| | | AATTTCAAAGATCAAGTCATTTTGCTGAATAAGCATATTGACGCATACAAAACATTCCC |
| | | ACCAACAGAGCCTAAAAAGGACAAAAAGAAGAAGGCTGATGAAACTCAAGCCTTACC |
| | | GCAGAGACAGAAGAAACAGCAAACTGTGACTCTTCTTCCTGCTGCAGATTTGGATGA |
| | | TTTCTCCAAACAATTGCAACAATCCATGAGCAGTGCTGACTCAACTCAGGCCTAAACT |
| | | CATGCAGACCACACAAGGCAGATGGGCTATATAAACGTTTTCGCTTTTCCGTTTACGA |
| | | TATATAGTCTACTCTTGTGCAGAATGAATTCTCGTAACTACATAGCACAAGTAGATGT |
| | | AGTTAACTTTAATCTCACATAGCAATCTTTAATCAGTGTGTAACATTAGGGAGGACTT |
| | | GAAAGAGCCACCACATTTTCACCGAGGCCACGCGGAGTACGATCGAGTGTACAGTG |
| | | AACAATGCTAGGGAGAGCTGCCTATATGGAAGAGCCCTAATGTGTAAAATTAATTTTA |
| | | GTAGTGCTATCCCCATGTGATTTTAATAGCTTCTTAGGAGAATGACAAAAAAAAAAAA |
| | | AAAAAAAAAAAAAAAAAAAAA |
| 9 | SARS-CoV | MFIFLLFLTLTSGSDLDRCTTFDDVQAPNYTQHTSSMRGVYYPDEIFRSDTLYLTQDLFLP |
| | spike protein | FYSNVTGFHTINHTFDNPVIPFKDGIYFAATEKSNVVRGWVFGSTMNNKSQSVIIINNSTN |
| | | VVIRACNFELCDNPFFAVSKPMGTQTHTMIFDNAFNCTFEYISDAFSLDVSEKSGNFKHL |
| | | REFVFKNKDGFLYVYKGYQPIDVVRDLPSGFNTLKPIFKLPLGINITNFRAILTAFSPAQDT |
| | | WGTSAAAYFVGYLKPTTFMLKYDENGTITDAVDCSQNPLAELKCSVKSFEIDKGIYQTSN |
| | | FRVVPSGDVVRFPNITNLCPFGEVFNATKFPSVYAWERKKISNCVADYSVLYNSTFFSTF |
| | | KCYGVSATKLNDLCFSNVYADSFVVKGDDVRQIAPGQTGVIADYNYKLPDDFMGCVLAW |
| | | NTRNIDATSTGNYNYKYRYLRHGKLRPFERDISNVPFSPDGKPCTPPALNCYWPLNDYG |
| | | FYTTTGIGYQPYRVVVLSFELLNAPATVCGPKLSTDLIKNQCVNFNFNGLTGTGVLTPSSK |
| | | RFQPFQQFGRDVSDFTDSVRDPKTSEILDISPCSFGGVSVITPGTNASSEVAVLYQDVNC |
| | | TDVSTAIHADQLTPAWRIYSTGNNVFQTQAGCLIGAEHVDTSYECDIPIGAGICASYHTVS |
| | | LLRSTSQKSIVAYTMSLGADSSIAYSNNTIAIPTNFSISITTEVMPVSMAKTSVDCNMYICG |
| | | DSTECANLLLQYGSFCTQLNRALSGIAAEQDRNTREVFAQVKQMYKTPTLKYFGGFNFS |
| | | QILPDPLKPTKRSFIEDLLFNKVTLADAGFMKQYGECLGDINARDLICAQKFNGLTVLPPLL |
| | | TDDMIAAYTAALVSGTATAGWTFGAGAALQIPFAMQMAYRFNGIGVTQNVLYENQKQIA |
| | | NQFNKAISQIQESLTTTSTALGKLQDVVNQNAQALNTLVKQLSSNFGAISSVLNDILSRLD |
| | | KVEAEVQIDRLITGRLQSLQTYVTQQLIRAAEIRASANLAATKMSECVLGQSKRVDFCGK |
| | | GYHLMSFPQAAPHGVVFLHVTYVPSQERNFTTAPAICHEGKAYFPREGVFVFNGTSWFI |
| | | TQRNFFSPQIITTDNTFVSGNCDVVIGIINNTVYDPLQPELDSFKEELDKYFKNHTSPDVDL |
| | | GDISGINASVVNIQKEIDRLNEVAKNLNESLIDLQELGKYEQYIKWPWYVWLGFIAGLIAIV |
| | | MVTILLCCMTSCCSCLKGACSCGSCCKFDEDDSEPVLKGVKLHYT |
| 10 | SARS-CoV-2 | MFLLTTKRTMFVFLVLLPLVSSQCVNLTTRTQLPPAYTNSFTRGVYYPDKVFRSSVLHST |
| | spike protein | QDLFLPFFSNVTWFHAIHVSGTNGTKRFDNPVLPFNDGVYFASTEKSNIIRGWIFGTTLDS |
| | | KTQSLLIVNNATNVVIKVCEFQFCNDPFLGVYYHKNNKSWMESEFRVYSSANNCTFEYV |
| | | SQPFLMDLEGKQGNFKNLREFVFKNIDGYFKIYSKHTPINLVRDLPQGFSALEPLVDLPIGI |
| | | NITRFQTLLALHRSYLTPGDSSSGWTAGAAAYYVGYLQPRTFLLKYNENGTITDAVDCAL |
| | | DPLSETKCTLKSFTVEKGIYQTSNFRVQPTESIVRFPNITNLCPFGEVFNATRFASVYAWN |
| | | RKRISNCVADYSVLYNSASFSTFKCYGVSPTKLNDLCFTNVYADSFVIRGDEVRQIAPGQ |
| | | TGKIADYNYKLPDDFTGCVIAWNSNNLDSKVGGNYNYLYRLFRKSNLKPFERDISTEIYQ |
| | | AGSTPCNGVEGFNCYFPLQSYGFQPTNGVGYQPYRVVVLSFELLHAPATVCGPKKSTN |
| | | LVKNKCVNFNFNGLTGTGVLTESNKKFLPFQQFGRDIADTTDAVRDPQTLEILDITPCSFG |
| | | GVSVITPGTNTSNQVAVLYQDVNCTEVPVAIHADQLTPTWRVYSTGSNVFQTRAGCLIGA |
| | | EHVNNSYECDIPIGAGICASYQTQTNSPRRARSVASQSIIAYTMSLGAENSVAYSNNSIAIP |
| | | TNFTISVTTEILPVSMTKTSVDCTMYICGDSTECSNLLLQYGSFCTQLNRALTGIAVEQDK |
| | | NTQEVFAQVKQIYKTPPIKDFGGFNFSQILPDPSKPSKRSFIEDLLFNKVTLADAGFIKQYG |
| | | DCLGDIAARDLICAQKFNGLTVLPPLLTDEMIAQYTSALLAGTITSGWTFGAGAALQIPFA |
| | | MQMAYRFNGIGVTQNVLYENQKLIANQFNSAIGKIQDSLSSTASALGKLQDVVNQNAQAL |
| | | NTLVKQLSSNFGAISSVLNDILSRLDKVEAEVQIDRLITGRLQSLQTYVTQQLIRAAEIRASA |
| | | NLAATKMSECVLGQSKRVDFCGKGYHLMSFPQSAPHGVVFLHVTYVPAQEKNFTTAPAI |
| | | CHDGKAHFPREGVFVSNGTHWFVTQRNFYEPQIITTDNTFVSGNCDVVIGIVNNTVYDPL |
| | | QPELDSFKEELDKYFKNHTSPDVDLGDISGINASVVNIQKEIDRLNEVAKNLNESLIDLQEL |
| | | GKYEQYIKWPWYIWLGFIAGLIAIVMVTIMLCCMTSCCSCLKGCCSCGSCCKFDEDDSEP |
| | | VLKGVKLHYT |
| 11 | SARS-CoV | SDLDRCTTFDDVQAPNYTQHTSSMRGVYYPDEIFRSDTLYLTQDLFLPFYSNVTGFHTIN |
| | spike protein S1 | HTFDNPVIPFKDGIYFAATEKSNVVRGWVFGSTMNNKSQSVIIINNSTNVVIRACNFELCD |
| | subunit | NPFFAVSKPMGTQTHTMIFDNAFNCTFEYISDAFSLDVSEKSGNFKHLREFVFKNKDGFL |
| | | YVYKGYQPIDVVRDLPSGFNTLKPIFKLPLGINITNFRAILTAFSPAQDTWGTSAAAYFVGY |
| | | LKPTTFMLKYDENGTITDAVDCSQNPLAELKCSVKSFEIDKGIYQTSNFRVVPSGDVVRF |
| | | PNITNLCPFGEVFNATKFPSVYAWERKKISNCVADYSVLYNSTFFSTFKCYGVSATKLND |
| | | LCFSNVYADSFVVKGDDVRQIAPGQTGVIADYNYKLPDDFMGCVLAWNTRNIDATSTGN |
| | | YNYKYRYLRHGKLRPFERDISNVPFSPDGKPCTPPALNCYWPLNDYGFYTTTGIGYQPY |
| | | RVVVLSFELLNAPATVCGPKLSTDLIKNQCVNFNFNGLTGTGVLTPSSKRFQPFQQFGRD |
| | | VSDFTDSVRDPKTSEILDISPCSFGGVSVITPGTNASSEVAVLYQDVNCTDVSTAIHADQL |
| | | TPAWRIYSTGNNVFQTQAGCLIGAEHVDTSYECDIPIGAGICASYHTVSLLR |
| 12 | SARS-CoV | RVVPSGDVVRFPNITNLCPFGEVFNATKFPSVYAWERKKISNCVADYSVLYNSTFFSTFK |
| | spike protein | CYGVSATKLNDLCFSNVYADSFVVKGDDVRQIAPGQTGVIADYNYKLPDDFMGCVLAWN |
| | RBD | TRNIDATSTGNYNYKYRYLRHGKLRPFERDISNVPFSPDGKPCTPPALNCYWPLNDYGF |
| | | YTTTGIGYQPYRVVVLSFELLNAPATVCGPKLSTDLIKNQCVNF |
| 13 | SARS-CoV-2 | SQCVNLTTRTQLPPAYTNSFTRGVYYPDKVFRSSVLHSTQDLFLPFFSNVTWFHAIHVSG |
| | spike protein S1 | TNGTKRFDNPVLPFNDGVYFASTEKSNIIRGWIFGTTLDSKTQSLLIVNNATNVVIKVCEF |
| | subunit | QFCNDPFLGVYYHKNNKSWMESEFRVYSSANNCTFEYVSQPFLMDLEGKQGNFKNLRE |
| | | FVFKNIDGYFKIYSKHTPINLVRDLPQGFSALEPLVDLPIGINITRFQTLLALHRSYLTPGDS |
| | | SSGWTAGAAAYYVGYLQPRTFLLKYNENGTITDAVDCALDPLSETKCTLKSFTVEKGIYQ |
| | | TSNFRVQPTESIVRFPNITNLCPFGEVFNATRFASVYAWNRKRISNCVADYSVLYNSASF |
| | | STFKCYGVSPTKLNDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGVEGFNCYFPLQS |
| | | YGFQPTNGVGYQPYRVVVLSFELLHAPATVCGPKKSTNLVKNKCVNFNFNGLTGTGVLT |
| | | ESNKKFLPFQQFGRDIADTTDAVRDPQTLEILDITPCSFGGVSVITPGTNTSNQVAVLYQD |
| | | VNCTEVPVAIHADQLTPTWRVYSTGSNVFQTRAGCLIGAEHVNNSYECDIPIGAGICASY |
| | | QTQTNSPRRAR |
| 14 | SARS-CoV-2 | RVQPTESIVRFPNITNLCPFGEVFNATRFASVYAWNRKRISNCVADYSVLYNSASFSTFK |
| | spike protein | CYGVSPTKLNDLCFTNVYADSFVIRGDEVRQIAPGQTGKIADYNYKLPDDFTGCVIAWNS |
| | RBD | NNLDSKVGGNYNYLYRLFRKSNLKPFERDISTEIYQAGSTPCNGVEGFNCYFPLQSYGF |
| | | QPTNGVGYQPYRVVVLSFELLHAPATVCGPKKSTNLVKNKCVNF |
| 15 | SARS-CoV-2 | PNITNLCPFGEVFNATRFASVYAWNRKRISNCVADYSVLYNSASFSTFKCYGVSPTKLND |
| | spike protein | LCFTNVYDSFVIRGDEVRQIAPGQTGKIADYNYKLPDDFTGCVIAWNSNNLDSKVGGNYN |
| | RBD | YLYRLFRKSNLKPFERDISTEIYQAGSTPCNGVEGFNCYFPLQSYGFQPTNGVGYQPYR |
| | | VVVLSFELLHAPATVCGPKKS |
| 16 | SARS-CoV-2 | MLLVNQSHQGFNKEHTSKMVSAIVLYVLLAAAAHSAFAQCVNLTTRTQLPPAYTNSFTRG |
| | spike protein S1 | VYYPDKVFRSSVLHSTQDLFLPFFSNVTWFHAIHVSGTNGTKRFDNPVLPFNDGVYFAST |
| | subunit | EKSNIIRGWIFGTTLDSKTQSLLIVNNATNVVIKVCEFQFCNDPFLGVYYHKNNKSWMESE |
| | | FRVYSSANNCTFEYVSQPFLMDLEGKQGNFKNLREFVFKNIDGYFKIYSKHTPINLVRDL |
| | | PQGFSALEPLVDLPIGINITRFQTLLALHRSYLTPGDSSSGWTAGAAAYYVGYLQPRTFLL |
| | | KYNENGTITDAVDCALDPLSETKCTLKSFTVEKGIYQTSNFRVQPTESIVRFPNITNLCPF |
| | | GEVFNATRFASVYAWNRKRISNCVADYSVLYNSASFSTFKCYGVSPTKLNDLCFTNVYA |
| | | DSFVIRGDEVRQIAPGQTGKIADYNYKLPDDFTGCVIAWNSNNLDSKVGGNYNYLYRLFR |
| | | KSNLKPFERDISTEIYQAGSTPCNGVEGFNCYFPLQSYGFQPTNGVGYQPYRVVVLSFE |
| | | LLHAPATVCGPKKSTNLVKNKCVNFNFNGLTGTGVLTESNKKFLPFQQFGRDIADTTDAV |
| | | RDPQTLEILDITPCSFGGVSVITPGTNTSNQVAVLYQDVNCTEVPVAIHADQLTPTWRVYS |
| | | TGSNVFQTRAGCLIGAEHVNNSYECDIPIGAGICASYQTQTNSPRRAR |
| 17 | SARS-CoV | MNITNLCPFGEVFNATKFPSVYAWERKKISNCVADYSVLYNSTFFSTFKCYGVSATKLND |
| | spike protein | LCFSNVYADSFVVKGDDVRQIAPGQTGVIADYNYKLPDDFMGCVLAWNTRNIDATSTGN |
| | RBD | YNYKYRYLRHGKLRPFERDISNVPFSPDGKPCTPPALNCYWPLNDYGFYTTTGIGYQPY |
| | | RVVVLSFELLNAPATYLSLNTAAAL |
| 18 | Artificial | MFIFLFFLTLVSGSTIEKCTTFDDVQPPNYTQFLSSHRGVYYPDDIFRSDVLHLTQDHFLP |
| | Sarbecovirus | FDSNVTGFITFGLNFDNPIIPFKDGIYFAATEKSNVIRGWVFGSTMNNKSQSVIIMNNSTNV |
| | spike consensus | VIRACNFELCDNPFFAVSKPNNTQIPSYIFNNAFNCTFEYVSDDFNLDVGEKPGNFKHLR |
| | 1 | EFVFKNKDGFLYVYSGYQPIDVASGLPSGFNALKPIFKLPLGINITNFRTLLTAFPPRPDYW |
| | | GTSAAAYFVGYLKPTTFMLKYDENGTITDAVDCSQNPLAELKCSVKSFEIDKGIYQTSNFR |
| | | VVPSGDVVRFPNITNLCPFGEVFNATKFPSVYAWERKRISNCVADYSVLYNSTFFSTFKC |
| | | YGVSATKLNDLCFSNVYADSFVVKGDDVRQIAPGQTGVIADYNYKLPDDFMGCVLAWNT |
| | | RNIDATSTGNYNYKYRYLRHGKLRPFERDISNVPFSPDGKPCTPPALNCYWPLNDYGFY |
| | | TTTGIGYQPYRVVVLSFELLNAPATVCGPKLSTDLIKNQCVNFNFNGLTGTGVLTPSSKRF |
| | | QPFQQFGRDVSDFTDSVRDPKTSEILDISPCSFGGVSVITPGTNTSSEVAVLYQDVNCTD |
| | | VPVAIHADQLTPAWRVYSTGNNVFQTQAGCLIGAEHVDTSYECDIPIGAGICASYHTVSSL |
| | | RSTSQKSIVAYTMSLGADSSIAYSNNTIAIPTNFSISITTEVMPVSMAKTSVDCNMYICGDS |
| | | TECANLLLQYGSFCTQLNRALSGIAVEQDRNTREVFAQVKQMYKTPTLKDFGGFNFSQIL |
| | | PDPLKPTKRSFIEDLLFNKVTLADAGFMKQYGECLGDINARDLICAQKFNGLTVLPPLLTD |
| | | DMIAAYTAALVSGTATAGWTFGAGAALQIPFAMQMAYRFNGIGVTQNVLYENQKQIANQ |
| | | FNKAISQIQESLTTTSTALGKLQDVVNQNAQALNTLVKQLSSNFGAISSVLNDILSRLDPPE |
| | | AEVQIDRLITGRLQSLQTYVTQQLIRAAEIRASANLAATKMSECVLGQSKRVDFCGKGYH |
| | | LMSFPQAAPHGVVFLHVTYVPSQERNFTTAPAICHEGKAYFPREGVFVFNGTSWFITQR |
| | | NFFSPQIITTDNTFVSGSCDVVIGIINNTVYDPLQPELDSFKEELDKYFKNHTSPDVDLGDI |
| | | SGINASVVNIQKEIDRLNEVAKNLNESLIDLQELGKYEQYIKWPWYVWLGFIAGLIAIVMVTI |
| | | LLCCMTSCCSCLKGACSCGSCCKFDEDDSEPVLKGVKLHYT |
| 19 | Artificial | MFIFLLFFTLTSGSDLDRCTTFDDVQAPNYTQHTSSMRGVYYPDEIFRSDTLYLTQDLFLP |
| | Sarbecovirus | FYSNVTGFHTINHTFDNPVIPFKDGIYFAATEKSNVVRGWVFGSTMNNKSQSVIIINNSTN |
| | spike consensus | VVIRACNFELCDNPFFVVSKPMGTRTHTMIFDNAFNCTFEYISDAFSLDVSEKSGNFKHL |
| | 2 | REFVFKNKDGFLYVYKGYQPIDVVRDLPSGFNTLKPIFKLPLGIKITNFRAILTAFSPAQGT |
| | | WGTSAAAYFVGYLKPTTFMLKYDENGTITDAVDCSQNPLAELKCSVKSFEIDKGIYQTSN |
| | | FRVVPSGDVVRFPNITNLCPFGEVFNATKFPSVYAWERKRISNCVADYSVLYNSTSFSTF |
| | | KCYGVSATKLNDLCFSNVYADSFVVKGDDVRQIAPGQTGVIADYNYKLPDDFMGCVLAW |
| | | NTRNIDATSTGNHNYKYRYLRHGKLRPFERDISNVPFSPDGKPCTPPAPNCYWPLRGYG |
| | | FYTTSGIGYQPYRVVVLSFELLNAPATVCGPKLSTDLIKNQCVNFNFNGLTGTGVLTPSSK |
| | | RFQPFQQFGRDVSDFTDSVRDPKTSEILDISPRSFGGVSVITPGTNASSEVAVLYQDVNC |
| | | TDVSTLIHAEQLTPAWRIYSTGNNVFQTQAGCLIGAEHVDTSYECDIPIGAGICASYHTVS |
| | | SLRSTSQKSIVAYTMSLGADSSIAYSNNTIAIPTNFSISITTEVMPVSMTKTSVDCNMYICG |
| | | DSTECANLLLQYGSFCRQLNRALSGIAAEQDRNTREVFVQVKQMYKTPTLKDFGGFNFS |
| | | QILPDPLKPTKRSFIEDLLFNKVTLADAGFMKQYGECLGDINARDLICAQKFNGLTVLPPLL |
| | | TVDMIAAYTAALVSGTATAGWTFGAGAALQIPFAMQMAYRFNGIGVTQNVLYENQKQIA |
| | | NQFNKAISQIQESLTTTSTALGKLQDVVNQNAQALNTLVKQLSSNFGAISSVLNDILSRLD |
| | | PPEAEVQIDRLITGRLQSLQTYVTQQLIRAAEIRASANLAATKMSECVLGQSKRVGFCGK |
| | | GYHLMSFPQAAPHGVVFLHVTYVPSQERNFTTAPAICHEGKAYFPREGVFVFSGTSWFI |
| | | TQRNFFSPQIITTDNTFVSGNCDVVIGIINNTVYDPLQPELDSFKEELDKYFKNHTSPDVDL |
| | | GDISGINASVVNIQEEIDRLNEVAKNLNESLIDLQELGKYEQYIKWPWYVWLGFIAGLIAIV |
| | | MVTILLCCMTSCCSCLKGACSCGSCCKFDEDDSEPVLKGVKLHYT |
| 20 | Artificial | MFIFLFFLTLVSGSDLESCTTFDDVQAPNYPQHSSSRRGVYYPDEIFRSDTLYLTQDLFLP |
| | Sarbecovirus | FDSNVTGFHTINHRFDNPVIPFKDGVYFAATEKSNVVRGWVFGSTMNNKSQSVIIINNST |
| | spike consensus | NVVIRACNFELCDNPFFAVSKPTGTQTHTMIFNNAFNCTFEYISDSFSLDVAEKSGNFKHL |
| | 3 | REFVFKNKDGFLYVYKGYQPIDVVRDLPSGFNILKPIFKLPLGINITNFRAILTAFLPAQDTW |
| | | GTSAAAYFVGYLKPATFMLKYDENGTITDAVDCSQNPLAELKCSVKSFEIDKGIYQTSNF |
| | | RVAPSKEVVRFPNITNLCPFGEVFNATTFPSVYAWERKRISNCVADYSVLYNSTSFSTFK |
| | | CYGVSATKLNDLCFSNVYADSFVVKGDDVRQIAPGQTGVIADYNYKLPDDFTGCVLAWN |
| | | TRNIDATQTGNYNYKYRSLRHGKLRPFERDISNVPFSPDGKPCTPPAFNCYWPLNDYGF |
| | | YTTNGIGYQPYRVVVLSFELLNAPATVCGPKLSTDLIKNQCVNFNFNGLTGTGVLTPSSK |
| | | RFQPFQQFGRDVSDFTDSVRDPKTSEILDISPCSFGGVSVITPGTNTSSEVAVLYQDVNC |
| | | TDVPVAIHADQLTPAWRIYSTGNNVFQTQAGCLIGAEHVDTSYECDIPIGAGICASYHTVS |
| | | SLRSTSQKSIVAYTMSLGADSSIAYSNNTIAIPTNFSISITTEVMPVSMAKTSVDCNMYICG |
| | | DSTECANLLLQYGSFCTQLNRALSGIAVEQDRNTREVFAQVKQMYKTPTLKDFGGFNFS |
| | | QILPDPLKPTKRSFIEDLLFNKVTLADAGFMKQYGECLGDINARDLICAQKFNGLTVLPPLL |
| | | TDDMIAAYTAALVSGTATAGWTFGAGAALQIPFAMQMAYRFNGIGVTQNVLYENQKQIA |
| | | NQFNKAISQIQESLTTTSTALGKLQDVVNQNAQALNTLVKQLSSNFGAISSVLNDILSRLD |
| | | PPEAEVQIDRLITGRLQSLQTYVTQQLIRAAEIRASANLAATKMSECVLGQSKRVDFCGK |
| | | GYHLMSFPQAAPHGVVFLHVTYVPSQERNFTTAPAICHEGKAYFPREGVFVFNGTSWFI |
| | | TQRNFFSPQIITTDNTFVSGNCDVVIGIINNTVYDPLQPELDSFKEELDKYFKNHTSPDVDL |
| | | GDISGINASVVNIQKEIDRLNEVAKNLNESLIDLQELGKYEQYIKWPWYVWLGFIAGLIAIV |
| | | MVTILLCCMTSCCSCLKGACSCGSCCKFDEDDSEPVLKGVKLHYT |
| 21 | Artificial | MFIFLFFLTLVSGSTIEKCTTFDDVQPPNYTQFLSSHRGVYYPDDIFRSDVLHLTQDHFLP |
| | Sarbecovirus | FDSNVTGFITFGLNFDNPIIPFKDGIYFAATEKSNVIRGWVFGSTMNNKSQSVIIMNNSTNV |
| | spike consensus | VIRACNFELCDNPFFAVSKPNNTQIPSYIFNNAFNCTFEYVSDDFNLDVGEKPGNFKHLR |
| | 4 | EFVFKNKDGFLYVYSGYQPIDVASGLPSGFNALKPIFKLPLGINITNFRTLLTAFPPRPDYW |
| | | GTSAAAYFVGYLKPTTFMLKYDENGTITDAVDCSQNPLAELKCSVKSFEIDKGIYQTSNFR |
| | | VAPSKEVVRFPNITNLCPFGEVFNATTFPSVYAWERKRISNCVADYSVLYNSTSFSTFKC |
| | | YGVSATKLNDLCFSNVYADSFVVKGDDVRQIAPGQTGVIADYNYKLPDDFMGCVLAWNT |
| | | RNIDATSTGNYNYKYRSLRHGKLRPFERDISNVPFSPDGKPCTPPAFNCYWPLNDYGFF |
| | | TTNGIGYQPYRVVVLSFELLNAPATVCGPKLSTDLIKNQCVNFNFNGLTGTGVLTPSSKR |
| | | FQPFQQFGRDVSDFTDSVRDPKTSEILDISPCSFGGVSVITPGTNTSSEVAVLYQDVNCT |
| | | DVPVAIHADQLTPAWRVYSTGNNVFQTQAGCLIGAEHVDTSYECDIPIGAGICASYHTVS |
| | | SLRSTSQKSIVAYTMSLGADSSIAYSNNTIAIPTNFSISITTEVMPVSMAKTSVDCNMYICG |
| | | DSTECANLLLQYGSFCTQLNRALSGIAVEQDRNTREVFAQVKQMYKTPTLKDFGGFNFS |
| | | QILPDPLKPTKRSFIEDLLFNKVTLADAGFMKQYGECLGDINARDLICAQKFNGLTVLPPLL |
| | | TDDMIAAYTAALVSGTATAGWTFGAGAALQIPFAMQMAYRFNGIGVTQNVLYENQKQIA |
| | | NQFNKAISQIQESLTTTSTALGKLQDVVNQNAQALNTLVKQLSSNFGAISSVLNDILSRLD |
| | | PPEAEVQIDRLITGRLQSLQTYVTQQLIRAAEIRASANLAATKMSECVLGQSKRVDFCGK |
| | | GYHLMSFPQAAPHGVVFLHVTYVPSQERNFTTAPAICHEGKAYFPREGVFVFNGTSWFI |
| | | TQRNFFSPQIITTDNTFVSGSCDVVIGIINNTVYDPLQPELDSFKEELDKYFKNHTSPDVDL |
| | | GDISGINASVVNIQKEIDRLNEVAKNLNESLIDLQELGKYEQYIKWPWYVWLGFIAGLIAIV |
| | | MVTILLCCMTSCCSCLKGACSCGSCCKFDEDDSEPVLKGVKLHYT |
| 22 | Artificial | MFVFLFLLPLVSSQCVNLTTRTGIPPGYTNSSTRGVYYPDKVFRSSVLHLTQDLFLPFFSN |
| | Sarbecovirus | VTWFNAINYQGGFKRFDNPVLPFNDGVYFASTEKSNIIRGWIFGTTLDARTQSLLIVNNAT |
| | spike consensus | NVVIKVCEFQFCTDPFLGVYYHNNNKTWVENEFRVYSSANNCTFEYVSQPFLMDLEGKQ |
| | 5 | GNFKNLREFVFKNVDGYFKIYSKHTPIDLVRDLPRGFSALEPLVDLPIGINITRFQTLLALH |
| | | RSYLTPGKLESGWTTGAAAYYVGYLQPRTFLLSYNENGTITDAVDCALDPLSETKCTLKS |
| | | LTVEKGIYQTSNFRVQPTISIVRFPNITNLCPFGEVFNATRFASVYAWNRKRISNCVADYS |
| | | VLYNSTSFSTFKCYGVSPTKLNDLCFTNVYADSFVVRGDEVRQIAPGQTGVIADYNYKLP |
| | | DDFTGCVIAWNSVKQDALTGGNYNYLYRLFRKSKLKPFERDISTEIYQAGSTPCNGVVGL |
| | | NCYYPLQRYGFHPTTGVGYQPYRVVVLSFELLNAPATVCGPKKSTNLVKNKCVNFNFNG |
| | | LTGTGVLTESNKKFLPFQQFGRDIADTTDAVRDPQTLEILDITPCSFGGVSVITPGTNTSN |
| | | QVAVLYQDVNCTEVPVAIHADQLTPTWRVYSTGSNVFQTRAGCLIGAEHVNNSYECDIPI |
| | | GAGICASYQTQTNSPRRARSVNSQSIIAYTMSLGAENSVAYSNNSIAIPTNFTISVTTEILP |
| | | VSMTKTSVDCTMYICGDSIECSNLLLQYGSFCTQLNRALTGIAVEQDKNTQEVFAQVKQI |
| | | YKTPPIKDFGGFNFSQILPDPSKPSKRSFIEDLLFNKVTLADAGFIKQYGDCLGDIAARDLI |
| | | CAQKFNGLTVLPPLLTDEMIAQYTSALLAGTITSGWTFGAGAALQIPFAMQMAYRFNGIG |
| | | VTQNVLYENQKLIANQFNSAIGKIQDSLSSTASALGKLQDVVNQNAQALNTLVKQLSSNF |
| | | GAISSVLNDILSRLDPPEAEVQIDRLITGRLQSLQTYVTQQLIRAAEIRASANLAATKMSEC |
| | | VLGQSKRVDFCGKGYHLMSFPQSAPHGVVFLHVTYVPAQEKNFTTAPAICHEGKAHFPR |
| | | EGVFVSNGTHWFVTQRNFYEPQIITTDNTFVSGSCDVVIGIVNNTVYDPLQPELDSFKEEL |
| | | DKYFKNHTSPDVDLGDISGINASVVNIQKEIDRLNEVAKNLNESLIDLQELGKYEQYIKWP |
| | | WYIWLGFIAGLIAIIMVTIMLCCMTSCCSCLKGCCSCGSCCKFDEDDSEPVLKGVKLHYT |
| 23 | Artificial | MFVFLFLLPLVSSQCVNLTTRTGIPPGYTNSSTRGVYYPDKVFRSSVLHLTQDLFLPFFSN |
| | Sarbecovirus | VTWFNAINYQGGFKRFDNPVLPFNDGVYFASTEKSNIIRGWIFGTTLDARTQSLLIVNNAT |
| | spike consensus | NVVIKVCEFQFCTDPFLGVYYHNNNKTWVENEFRVYSSANNCTFEYVSQPFLMDLEGKQ |
| | 6 | GNFKNLREFVFKNVDGYFKIYSKHTPIDLVRDLPRGFSALEPLVDLPIGINITRFQTLLALH |
| | | RSYLTPGKLESGWTTGAAAYYVGYLQPRTFLLSYNENGTITDAVDCALDPLSETKCTLKS |
| | | FTVEKGIYQTSNFRVQPTESIVRFPNITNLCPFGEVFNATRFASVYAWNRKRISNCVADYS |
| | | VLYNSASFSTFKCYGVSPTKLNDLCFTNVYADSFVIRGDEVRQIAPGQTGKIADYNYKLP |
| | | DDFTGCVIAWNSNNLDSKVGGNYNYLYRLFRKSNLKPFERDISTEIYQAGSTPCNGVEG |
| | | FNCYFPLQSYGFQPTNGVGYQPYRVVVLSFELLHAPATVCGPKKSTNLVKNKCVNFNFN |
| | | GLTGTGVLTESNKKFLPFQQFGRDIADTTDAVRDPQTLEILDITPCSFGGVSVITPGTNTS |
| | | NQVAVLYQDVNCTEVPVAIHADQLTPTWRVYSTGSNVFQTRAGCLIGAEHVNNSYECDI |
| | | PIGAGICASYQTQTNSPRRARSVNSQSIIAYTMSLGAENSVAYSNNSIAIPTNFTISVTTEIL |
| | | PVSMTKTSVDCTMYICGDSIECSNLLLQYGSFCTQLNRALTGIAVEQDKNTQEVFAQVKQ |
| | | IYKTPPIKDFGGFNFSQILPDPSKPSKRSFIEDLLFNKVTLADAGFIKQYGDCLGDIAARDLI |
| | | CAQKFNGLTVLPPLLTDEMIAQYTSALLAGTITSGWTFGAGAALQIPFAMQMAYRFNGIG |
| | | VTQNVLYENQKLIANQFNSAIGKIQDSLSSTASALGKLQDVVNQNAQALNTLVKQLSSNF |
| | | GAISSVLNDILSRLDPPEAEVQIDRLITGRLQSLQTYVTQQLIRAAEIRASANLAATKMSEC |
| | | VLGQSKRVDFCGKGYHLMSFPQSAPHGVVFLHVTYVPAQEKNFTTAPAICHEGKAHFPR |
| | | EGVFVSNGTHWFVTQRNFYEPQIITTDNTFVSGSCDVVIGIVNNTVYDPLQPELDSFKEEL |
| | | DKYFKNHTSPDVDLGDISGINASVVNIQKEIDRLNEVAKNLNESLIDLQELGKYEQYIKWP |
| | | WYIWLGFIAGLIAIIMVTIMLCCMTSCCSCLKGCCSCGSCCKFDEDDSEPVLKGVKLHYT |
| 24 | Artificial | MFVFLFLLPLVSSQCVNLTTRTGIPPGYTNSSTRGVYYPDKVFRSSILHLTQDLFLPFFSN |
| | Sarbecovirus | VTWFNTINYQGGFKKFDNPVLPFNDGVYFASTEKSNIIRGWIFGTTLDNTTQSLLIVNNAT |
| | spike consensus | NVVIKVCEFQFCTDPFLGVYYHNNNKTWVENEFRVYSSANNCTFEYVSQPFLMDLEGKQ |
| | 7 | GNFKNLREFVFKNVDGYFKIYSKHTPIDLVRDLPRGFSALEPLVDLPIGINITRFQTLLALH |
| | | RSYLTPGKLESGWTTGAAAYYVGYLQQRTFLLSYNENGTITDAVDCSLDPLSETKCTLKS |
| | | LTVEKGIYQTSNFRVQPTISIVRFPNITNLCPFGEVFNATKFASVYAWNRKRISNCVADYS |
| | | VLYNSTSFSTFKCYGVSPTKLNDLCFTNVYADSFVVRGDEVRQIAPGQTGVIADYNYKLPDD |
| | | FTGCVIAWNSVKQDAKTGGNYNYLYRLFRKSKLKPFERDISTEIYQAGSTPCNGVVGLNCYY |
| | | PLERYGFHPTNGVGYQPYRVVVLSFELLNAPATVCGPKLSTTLVKNKCVNFNFNGLTGTGV |
| | | LTTSKKKFLPFQQFGRDISDTTDAVRDPQTLEILDITPCSFGGVSVITPGTNTSNQVAVLY |
| | | QDVNCTEVPMAIHADQLTPTWRVYSTGANVFQTRAGCLIGAEHVNNSYECDIPIGAGICA |
| | | SYHTQTNSRSVNSQSIIAYTMSLGAENSVAYSNNSIAIPTNFTISVTTEILPVSMTKTSVDC |
| | | TMYICGDSIECSNLLLQYGSFCTQLNRALTGIAVEQDKNTQEVFAQVKQIYKTPPIKDFGG |
| | | FNFSQILPDPSKPSKRSFIEDLLFNKVTLADAGFIKQYGDCLGDIAARDLICAQKFNGLTVL |
| | | PPLLTDEMIAQYTSALLAGTITSGWTFGAGAALQIPFAMQMAYRFNGIGVTQNVLYENQK |
| | | LIANQFNSAIGKIQDSLSSTASALGKLQDVVNQNAQALNTLVKQLSSNFGAISSVLNDILSR |
| | | LDPPEAEVQIDRLITGRLQSLQTYVTQQLIRAAEIRASANLAATKMSECVLGQSKRVDFCG |
| | | KGYHLMSFPQSAPHGVVFLHVTYVPAQEKNFTTAPAICHEGKAHFPREGVFVSNGTHW |
| | | FVTQRNFYEPQIITTDNTFVSGSCDVVIGIVNNTVYDPLQPELDSFKEELDKYFKNHTSPD |
| | | VDLGDISGINASVVNIQKEIDRLNEVAKNLNESLIDLQELGKYEQYIKWPWYIWLGFIAGLIA |
| | | IIMVTIMLCCMTSCCSCLKGCCSCGSCCKFDEDDSEPVLKGVKLHYT |
| 25 | Artificial | MFIFLFFLTLVSSQCVNFDDRTPPPPTQTNSSTRGVYYPDDIFRSSVLHLTQDLFLPFFSN |
| | Sarbecovirus | VTWFHTINYRFDNPVIPFKDGVYFAATEKSNVIRGWIFGSTMDNKSQSVIIVNNSTNVVIR |
| | spike consensus | VCNFQLCDNPFFAVYNKTWTQIESRVYSNAFNCTFEYVSKSFLLDLGEKQGNFKHLREF |
| | 8 | VFKNKDGFLKVYSGYTPIDLVRDLPSGFNALKPIFKLPLGINITNFRTLLTAFRPGQSGWG |
| | | TSAAAYFVGYLKPRTFMLKYDENGTITDAVDCSQDPLAELKCTLKSFEVEKGIYQTSNFR |
| | | VQPTKEVVRFPNITNLCPFGEVFNATTFPSVYAWERKRISNCVADYSVLYNSTSFSTFKC |
| | | YGVSPTKLNDLCFSNVYADSFVVKGDDVRQIAPGQTGVIADYNYKLPDDFTGCVLAWNT |
| | | RNIDATTGGNYNYLYRLFRHSKLKPFERDISNVIYSPGGKPCTPPGFNCYWPLNDYGFYP |
| | | TNGVGYQPYRVVVLSFELLNAPATVCGPKLSTDLVKNKCVNFNFNGLTGTGVLTPSSKR |
| | | FQPFQQFGRDVSDFTDSVRDPKTLEILDISPCSFGGVSVITPGTNTSSEVAVLYQDVNCT |
| | | DVPVAIHADQLTPAWRVYSTGNNVFQTQAGCLIGAEHVNNSYECDIPIGAGICASYHTVS |
| | | SLRSTSQKSIVAYTMSLGAENSIAYSNNSIAIPTNFSISVTTEVLPVSMTKTSVDCTMYICG |
| | | DSTECSNLLLQYGSFCTQLNRALSGIAVEQDKNTQEVFAQVKQIYKTPPIKDFGGFNFSQI |
| | | LPDPSKPSKRSFIEDLLFNKVTLADAGFMKQYGDCLGDINARDLICAQKFNGLTVLPPLLT |
| | | DEMIAAYTAALVSGTATAGWTFGAGAALQIPFAMQMAYRFNGIGVTQNVLYENQKQIAN |
| | | QFNKAISQIQESLTTTSTALGKLQDVVNQNAQALNTLVKQLSSNFGAISSVLNDILSRLDP |
| | | PEAEVQIDRLITGRLQSLQTYVTQQLIRAAEIRASANLAATKMSECVLGQSKRVDFCGKG |
| | | YHLMSFPQAAPHGVVFLHVTYVPSQERNFTTAPAICHEGKAHFPREGVFVSNGTHWFIT |
| | | QRNFYSPQIITTDNTFVSGSCDVVIGIVNNTVYDPLQPELDSFKEELDKYFKNHTSPDVDL |
| | | GDISGINASVVNIQKEIDRLNEVAKNLNESLIDLQELGKYEQYIKWPWYVWLGFIAGLIAIV |
| | | MVTIMLCCMTSCCSCLKGCCSCGSCCKFDEDDSEPVLKGVKLHYT |
| 26 | Artificial | SNFRWVPSGDVVRFPNITNLCPFGEVFNATKFPSVYAWERKRISNCVADYSVLYNSTSFS |
| | Sarbecovirus | TFKCYGVSATKLNDLCFSNVYADSFVVKGDDVRQIAPGQTGVIADYNYKLPDDFMGCVL |
| | RBD consensus | AWNTRNIDATSTGNHNYKYRYLRHGKLRPFERDISNVPFSPDGKPCTPPAPNCYWPLR |
| | 1 | GYGFYTTSGIGYQPYRVVVLSFELLNAPATVCGPKLSTDLIKNQCVNFNFNGLTGTGVLT |
| | | PSSKRFQPFQQFGRDVSDFTDSVRDPKTSEIL |
| 27 | Artificial | SNFRVAPSKEVVRFPNITNLCPFGEVFNATTFPSVYAWERKRISNCVADYSVLYNSTSFS |
| | Sarbecovirus | TFKCYGVSATKLNDLCFSNVYADSFVVKGDDVRQIAPGQTGVIADYNYKLPDDFTGCVLA |
| | RBD consensus | WNTRNIDATQTGNYNYKYRSLRHGKLRPFERDISNVPFSPDGKPCTPPAFNCYWPLNDY |
| | 2 | GYTTNGIGYQPYRVVVLSFELLNAPATVCGPKLSTDLIKNQCVNFNFNGLTGTGVLTPSS |
| | | RFQPFQQFGRDVSDFTDSVRDPKTSEIL |
| 28 | Artificial | SNFRVQPTISIVRFPNITNLCPFGEVFNATRFASVYAWNRKRISNCVADYSVLYNSTSFST |
| | Sarbecovirus | FKCYGVSPTKLNDLCFTNVYADSFVVRGDEVRQIAPGQTGVIADYNYKLPDDFTGCVIAW |
| | RBD consensus | NSVKQDALTGGNYNYLYRLFRKSKLKPFERDISTEIYQAGSTPCNGVVGLNCYYPLQRY |
| | 3 | GFHPTTGVGYQPYRVVVLSFELLNAPATVCGPKKSTNLVKNKCVNFNFNGLTGTGVLTE |
| | | SNKKFLPFQQFGRDIADTTDAVRDPQTLEIL |
| 29 | Artificial | SNFRVQPTISIVRFPNITNLCPFGEVFNATKFASVYAWNRKRISNCVADYSVLYNSTSFST |
| | Sarbecovirus | FKCYGVSPTKLNDLCFTNVYADSFVVRGDEVRQIAPGQTGVIADYNYKLPDDFTGCVIAW |
| | RBD consensus | NSVKQDAKTGGNYNYLYRLFRKSKLKPFERDISTEIYQAGSTPCNGVVGLNCYYPLERY |
| | 4 | GFHPTNGVGYQPYRVVVLSFELLNAPATVCGPKLSTTLVKNKCVNFNFNGLTGTGVLTT |
| | | SKKKFLPFQQFGRDISDTTDAVRDPQTLEIL |
| 30 | Artificial | SNFRVQPTKEVVRFPNITNLCPFGEVFNATTFPSVYAWERKRISNCVADYSVLYNSTSFS |
| | Sarbecovirus | TFKCYGVSPTKLNDLCFSNVYADSFVVKGDDVRQIAPGQTGVIADYNYKLPDDFTGCVLA |
| | RBD consensus | WNTRNIDATTGGNYNYLYRLFRHSKLKPFERDISNVIYSPGGKPCTPPGFNCYWPLNDY |
| | 5 | GFYPTNGVGYQPYRVVVLSFELLNAPATVCGPKLSTDLVKNKCVNFNFNGLTGTGVLTP |
| | | SSKRFQPFQQFGRDVSDFTDSVRDPKTLEIL |
| 31 | Antibody 1 | GAGGTGCAGCTGGTGGAGTCTGGGGGAGGCTTGGTACAGCCTGGGGGGTCCCTGA |
| | (SS6V1-B5) VH | GACTCTCCTGTGCAGCCTCTGGATTCATCCTCAGGAACTACGAGATGCAGTGGGTC |
| | NT sequence | CGCCAAGCTCCAGGAAAAGGTCTGGAGTGGGTCTCAGTAATTGGTAATACGGGTGG |
| | | CACATTCTATGCAGGCTCCGTGAAGGGCCGATTCACCATCTCCAGAGAAAATGCCAA |
| | | GAACTCCTTGTATCTTCAAATGAATAGCCTGAGAGCCGAGGACACGGCCTTGTATTA |
| | | CTGTGCAAGAGTAACCTATACCAGCAGCCCTCTTCCATTCTGGTTCCTCGATCTCTG |
| | | GGGCCGTGGCACCCTGGTCACCGTCTCTTCA |
| 32 | Antibody 1 | GACATCCAGATGACCCAGTCTCCATCCTCCCTGTCTGCATCTGTAGGAGACAGAGTC |
| | (SS6V1-B5) VL | ACCATCACTTGCCGGGCAAGTCAGAGCATTGGCAACTATTTAAATTGGTATCAGCAG |
| | NT sequence | AAATCAGGGAAAGCCCCTAAGATCCTGATCTATGCTGCATCCAGTTTGCAAAGTGGG |
| | | GTCCCATCAAGGTTCAGTGGCAGTGGATCTGGGACAGATTTCACTCTCACCATCAGC |
| | | AGTCTGCAACCTGAAGATTTTGCAACTTACTATTGTCAACAGACGTATCGTACCCCT |
| | | CCGGAGGACAGTTTTGGCCAGGGGACCAAGGTGGAAATCAAA |
| 33 | Antibody 2 | GAGGTGCAGCTGGTGGAGTCTGGGGGAGGCTTGGTCCAGCCTGGGGGGTCCCTGA |
| | (SS6V2-G1) VH | GACTCTCCTGTGCAGCCTCTGGATTCACCGTCAGTAGCAACTACATGAGCTGGGTC |
| | NT sequence | CGCCAGGCTCCAGGGAAGGGGCTGGAGTGGGTCTCAGTTATTTATAGCGGTGGTA |
| | | GCACATTCTACGCAGACTCCGTGAAGGGCAGATTCACCATCTCCAGAGACAATTCCA |
| | | AGAACACGCTGTATCTTCAAATGAATAGCCTGAGAGCCGAGGACACGGCTGTCTATT |
| | | ACTGTGCGAGGGAGTTAAGGCACTACTTTGACTACTGGGGCCAGGGAACCCTGGT |
| | | CACCGTCTCTTCA |
| 34 | Antibody 2 | GACATCCAGTTGACCCAGTCTCCATCCTTCCTGTCTGCATCTGTAGGAGACAGAGTC |
| | (SS6V2-G1) VL | ACCATCACTTGCCGGGCCAGTCAGGGCATTAGCAGTTATTTAGCCTGGTATCAGCAA |
| | NT sequence | AAACCAGGGAAAGCCCCTAAGCTCCTGATCTATGCTGCATCAACTTTGCAAAGTGGG |
| | | GTCCCATCAAGGTTCAGCGGCAGTGGATCTGGGACAGAATTCACTCTCACAATCAGC |
| | | AGCCTGCAGCCTGAAGATTTTGCAACTTATTACTGTCAACAGCTTAATAGTTACCCTC |
| | | CGTACAGTTTTGGCCAGGGGACCAAGGTGGAAATCAAA |
| 35 | Antibody 3 | CAGGTTCAGCTGGTGCAGTCTGGAGCTGAGGTGAAGAGGCCTGGGGCCTCAGTGAA |
| | (SS6V3-G2) VH | GGTCTCCTGCAAGGCCTCTGGTTACAGCTTTACCAACTCTGGAATCACCTGGGTGC |
| | NT sequence | GACAGGCCCCTGGACAAGGGCTTGAGTGGATGGGCGGGACCAACTTTTATAATGGT |
| | | ATCACAAATTATCCACAGAACATCCAGGGCAGAGTCACCATGACTACAGACACGTCC |
| | | ACGAATACAGCCTATCTGGAGCTGACGAGCCTGAGATCTGAAGACACGGCCATGTAT |
| | | TACTGTGCGTTAAATCGTGTCGCGATTTTCAACGATGGTTACAACCCCTTGGGGTAC |
| | | TGGGGCCAGGGAACCCTGGTCACCGTCTCCTCA |
| 36 | Antibody 3 | GACATCGTGATGACCCAGTCTCCAGACTCCCTGGCTGTGTCTCTGGGCGAGAGGGC |
| | (SS6V3-G2) VL | CACCATCAACTGCAAGTCCAGCCAGAGTGTTTTATACAGTTCCAACAATAAGAACTA |
| | NT sequence | CTTAGCTTGGTACCAGCAGAAACCAGGACAGCCTCCTAAACTGCTCATTTACTGGGC |
| | | TTCTTCCCGGGAATCCGGGGTCCCGGACCGATTCAGTGGCAGCGGGTCTGGGACA |
| | | GATTTCACTCTCACCATCGACAGGCTGCAGGCTGAAGATGTGGCAATTTATTACTGT |
| | | CAGCAATATTTTAGTTCTCCCTTCTCTTTCGGCGGAGGGACCAAGGTGGAAATCAAA |
| 37 | Antibody 4 | GAGGTGCAGCTGGTGCAGTCTGGAGCAGAGGTGAAAAAGCCCGGGGAGGCTCTGA |
| | (SS6V4-A1) VH | AGATCTCCTGTCAGGGTTCTGGATACACCTTTAGTATGTACTGGATCGCCTGGGTGC |
| | NT sequence | GCCAGATGCCCGGGAAAGGCCTGGAGTGGATGGGAATCATCTATCCTGATGACTCT |
| | | GATAGAAGATATAGTCCGTCCTTTCAAGGCCAGGTCACCATCTCAGTCGACAAGTCC |
| | | ATCAGCACCGCCTACCTACAGTGGAGCAGCCTGAAGGCCTCGGACACCGCCATATA |
| | | TTACTGTGCGAGACTCCAGAATGGATATAGTTATGGCCTCCTTGAAAATTGGGGCC |
| | | AGGGAACCCTGGTCACCGTCTCCTCA |
| 38 | Antibody 4 | GACATCGTGATGACCCAGTCTCCAGACTCCCTGACTGTGTCTCTGGGCGAGAGGGC |
| | (SS6V4-A1) VL | CACCATCAACTGCAAGTCCAGCCAGAGTACTTTATACAGGTCCAACAATAAGAATTA |
| | NT sequence | CTTAGCTTGGTACCAGCAGAAGCCAGGACAGCCTCCTAAACTTCTCATTAACTGGGC |
| | | ATCTACCCGGGAATACGGGGTCCCTGATCGATTCAGTGGCAGCGGGTCTGGGACAG |
| | | ATTTCACTCTCACCATCAGCAGCCTGCAGGCTGAAGATGTGGCAGTTTACTACTGTC |
| | | AGCAATATTATAGTTATCCGTGGACGTTCGGCCAAGGGACCAAGGTGGAGATCAAA |
| 39 | Antibody 5 | GAGGTGCAGCTGGTGCAGTCTGGCGCAGAGGTGAGAAAGCCCGGGGAGTCTCTGA |
| | (SS6V5-C3) VH | AGATCTCCTGTAAGACTTCTGGATACACCTTTACCCACTATTGGATCGGCTGGGTGC |
| | NT sequence | GCCAGCTGCCCGGAAAAGGCCTCGAGTGGATGGGCATCATCTATCCTGATGACTCT |
| | | GATACCAAATACAGTCCGTCCTTCCAAGGCCAAATCACCATCTCAGCCGACAAGTCC |
| | | ATCAGCACCGCCTACCTGCAGTGGAACAGCCTGAAGGCCTCGGACACCGCCATGTA |
| | | TTTCTGTGCGACCGCGGATATTGTGGTGGGGTCCAACTTCTTTGACCACTGGGGCC |
| | | AGGGAACGCTGGTCACCGTCTCCTCA |
| 40 | Antibody 5 | GACATCCAGATGACCCAGTCTCCTTCCACCCTGTCTGCGTCTGTTGGAGACAGAGTC |
| | (SS6V5-C3) VL | ACCATCACTTGCCGGGCCAGTCAGAGTATTAGTACCTGGTTGGCCTGGTATCAGCA |
| | NT sequence | GAAACCAGGGAAACCCCCTAAGGTCCTGATCTATAAGGCGTCTATTTTAGAAAGTGG |
| | | GGTCTCATCAAGATTCAGCGGCAGTGGATCTGGGACAGAATTCACTCTCACCATCAG |
| | | CAGCCTGCAGCCTGATGATTTTGCAACTTATTACTGCCAACACTATAATAGTTATATA |
| | | AAAACATTCGGCCAAGGGACCAAGGTGGAAATCAAA |
| 41 | Antibody 6 | GAGGTGCAGCTGGTGGAGTCTGGGGGAGGCTTGGTCCAGCCTGGGGGGTCCCTGA |
| | (SS6V6-C4) VH | GACTCTCCTGTTCAGCCTCTGGATTCACCTTCAATACCTATGCTATGCACTGGGTCC |
| | NT sequence | GCCAGGCTCCAGGGAAGGGACCAGAATATGTTTCAGGTATTAGTTCTAATGGGGGT |
| | | ATCACAGACTACGCAGACTCCGTGAAGGGCAGATTCACCATCTCCAGAGACAATTCC |
| | | AAGAACACGCTGTATCTTCAAATGAGCAGTCTGAGACCTGAGGACACGGCTGTGTAT |
| | | TACTGTGTGAAAGACTCCCTGGCTACGGTGGTAACTCTCTTATCCTACTGGGGCCA |
| | | GGGAACCCTGGTCACCGTCTCTTCA |
| 42 | Antibody 6 | GACATCCAGATGACCCAGTCTCCATCCTCCCTGTCTGCATCTGTAGGAGACAGAGTC |
| | (SS6V6-C4) VL | ACCATCACTTGCCGGGCAAGTCAGACCATTAGCAGCTATTTAAATTGGTTTCAGCAG |
| | NT sequence | AAACCAGGTAAAGCCCCTAAACTCCTGATCTATGCTGCATCCAGTTTGCAAAGTGGG |
| | | GTCCCATCAAGGTTCAGTGGCAGTGGATCTGGGACAGATTTCACTCTCACCATCAGC |
| | | AGTCTGCAACCTGAAGATTTTGCCATTTACTACTGTCAACAGAGTTACAGTACCCCT |
| | | GGGACGTTCGGCCAAGGGACCAAGGTGGAAATCAAA |
| 43 | Antibody 7 | GAGGTGCAGCTGGTGCAGTCTGGAGCAGAGGTGAAAAAGCCCGGGGAGTCTCTGA |
| | (SS6V7-C5) VH | AGATCTCCTGTAAGGGTTCTGAAAACATCTTTTCCGGCTACTGGATCGGCTGGGTGC |
| | NT sequence | GCCAGATGCCCGGGAAAGGCCTGGAGTGGATGGGGATCATCTATCCTGATGACTCT |
| | | GATACCAGATACAACCCGTCCTTCCAAGGCCAGGTCACCATCTCAGCCGACCAGTC |
| | | CATCAGCACCGCCTACCTGCAGTGGAGCAGACTGAAGGCCTCGGACACCGCCATGT |
| | | ATTACTGTGCGAGACATTTAGGGGGTGGCAGCAGTTGGCCCATTGACTACTGGGGC |
| | | CAGGGAACCCTGGTCACCGTCTCCTCA |
| 44 | Antibody 7 | GACATCCAGATGACCCAGTCTCCATCCTCACTGTCTGCATCTGTAGGAGACAGAGTC |
| | (SS6V7-C5) VL | ACCATCACTTGTCGGGCGAATCAGGGCATTAGCAATTATTTAGCCTGGTTTCAGCAG |
| | NT sequence | AAACCAGGGAAAGCCCCTAAGTCCCTGATCTATGCTGCATCCAGTTTGCAAAGTGGG |
| | | GTCCCATCAAAGTTCAGCGGCAATAGATCTGGGACAGATTTCACTCTCACCATCAGC |
| | | AGCCTACAGCCTGAAGATTTTGCAACTTATTACTGCCAACAGTATAGTAGTTACCCC |
| | | TTCACTTTCGGCGGAGGGACCAAGGTGGAGATCAAA |
| 45 | Antibody 8 | GAGGTGCAGCTGGTGGAGTCTGGGGGAGACTTGGTCCAGCCTGGGGGGTCCCTGA |
| | (SS6V8-D3) VH | GACTCTCCTGTTCAGCCTCTGGATTCACCTTCAGCACCTATGCTATGCACTGGGTCC |
| | NT sequence | GCCAGGCTCCAGGGAAGGGACTGGATTATGTGGCATCCATTGCTAGTGATGGGGGT |
| | | ATCACAACTTACGCAGACTCCGTGAAGGGCAGATTCACCATCTCCAGAGACAATTCC |
| | | AAGAACGCGCTGTATCTTCAAATGAGTAGTCTGAGAGTTGAGGACACGGCTGTGTAT |
| | | TACTGTGTGAAAGATTCCTTGACTTCGGTGACTACGATTTTTGACTGCTGGGGCCAG |
| | | GGAACCCTGGTCACCGTCTCTTCA |
| 46 | Antibody 8 | GACATCCAGATGACCCAGTCTCCATCCTCCCTGTCTGCATCTGTAGGAGACAGCGTC |
| | (SS6V8-D3) VL | ACCATCACTTGCCGGGCAAGTCAGAACATTAACAGTTATTTACATTGGTATCAGCAG |
| | NT sequence | AAGCCAGGGAAAGCCCCTAAGCTCCTGATCTATACTGCATCCAGTTTGCAAAGTGGG |
| | | GTCCCATCAAGGTTCAGTGCCAGTGGATCTGGGACACATTTCACTCTCACCATCAGC |
| | | AGTCTGCAACCTGAAGATTTTGCAACTTACTACTGTCAACAGAGTTACACTGACCCC |
| | | TACACTTTTGGCCAGGGGTCCAAGGTGGAAATCAAA |
| 47 | Antibody 9 | CAGGTGCAGCTGCAGGAGTCGGGCCCAGGACTGGTGAAGCCTTCGGGGACCCTGT |
| | (SS6V9-D11) | CCCTCACCTGCGCTGTCTCTGGTGGCTCCATCAGCAGTAATATCTGGTGGAGTTGG |
| | VH NT | GTCCGCCAGCCCCCCGGAAAGGGGCTGGAGTGGATTGGGGACATCTATCACAGTG |
| | sequence | GGAGTACCAACTACAACCCGTCCCTCGAGGGTCGAGTCACCATATCAATAGACAAGT |
| | | CCAAGAACCAATTCTCCCTGAGGCTGACGTCTCTGACCGCCGCGGACACGGCCGTG |
| | | TATTACTGTGCGAGAGCGATTTCGCAGCAGTACTTTGATAGCAGTGTCCTGGGCTA |
| | | CTGGGGCCAGGGAATTCTGGTCACCGTCTCCTCA |
| 48 | Antibody 9 | GAAATAGTGATGACGCAGTCTCCAGCCACCCTGTCTGTGTCTCCAGGGGAAAGAGT |
| | (SS6V9-D11) VL | CACCCTCTCCTGCAGGGCCAGTCAGAGTGTTGTCACCAACTTAGCCTGGTACCAGC |
| | NT sequence | AGAAACCTGGCCAGGCTCCCAGGCTCCTCATCTATGGTGCTTCCACCAGGGCCACT |
| | | GGTATCCCAGCCAGGTTCAGTGGCAGTGGGTCTGGGACAGAGTTCACTCTCACCAT |
| | | CAGCAGCCTGCAGTCTGAAGATTTTGCAATTTATCACTGTCAGCAGTATAATAACTG |
| | | GCCTGGGTACACTTTTGGCCAGGGGACCAAAGTGGATATCAAA |
| 49 | Antibody 10 | GAGGTGCAGCTGGTGCAGTCTGGAGCAGAGGTGAAAAAGCCCGGGGAGTCTCTGA |
| | (SS6V10-E1) | AGATCTCCTGTCAGGGTTCTGAAGACAGCTTTACCGGCTACTGGATCGGTTGGGTG |
| | VH NT | CGCCAGCTGCCCGGGAAAGGCCTGGAGTGGATGGGGTTCATCTATCCAGATGACG |
| | sequence | GCGATACCAGATACAGCCCGGCCTTCCAAGGCCTGGTCACCTTCTCAGCCGACAAG |
| | | TCCATCACCACCGCCTACCTGCAGTGGAGTAGTTTAAAGGCCTCGGACACCGCCATA |
| | | TATTTCTGTGCGAGACATTTAGGGGGAGGCAGCAGCTGGCCCATTGACTCCTGGG |
| | | GCCAGGGAACCCAGGTCACCGTCTCCTCA |
| 50 | Antibody 10 | GACATCCAGATGACCCAGTCTCCATCCTCACTGTCTGCATCTGTAGGAGACAGAGTC |
| | (SS6V10-E1) VL | ACTATCACTTGTCGGGCGAGTCAGGGCATTAGAAATTATTTAGCCTGGTTTCAGCAG |
| | NT sequence | AAACCAGGGAAAGCCCCTAAGTCCCTGATCTATGCTGCATCCAGTTTGCAAAGTGGG |
| | | GTCCCATCAAAGTTCAGCGGCAGTGGATATGGGACAGACTTCACTCTCACCATCAAC |
| | | AGCCTGCAGCCTGAAGATTTTGCAACTTATTACTGCCAACAGTATAATAATCACCCC |
| | | TTCACTTTCGGCGGAGGGACCAAGGTGGAAATCAAA |
| 51 | Antibody 11 | CAGGTGCAGCTGCAGGAGTCGGGCCCAGGACTGGTGAAGCCTTCGGAGACCCTGT |
| | (SS6V11-E7) | CCCTCACTTGCACTGTCTCTGGTGGCTTCATCGGTCCTCACTACTGGAGCTGGGTCC |
| | VH NT | GCCAGCCCCCAGGGAAGGGACTGGAGTGGATTGGTTATATCTATATCAGCGGGAGC |
| | sequence | ACCAACTACAATCCCTCCCTCAAGAGTCGACTCACTATATCAGTAGACATGTCCAAGA |
| | | GCCAGTTCTCCCTGACACTGAGCTCTGCGACCGCTGCGGACACGGCCGTCTATTAC |
| | | TGTGCGAGAGGGGGGGGATATTTGGAAACCGGCCCCTTTGAGTACTGGGGCCAGG |
| | | GAACCCTGGTCACCGTCTCCTCA |
| 52 | Antibody 11 | GATATTGTGATGACTCAGTCTCCACTCTCCCTGCCCGTCACCCCTGGAGAGCCGGC |
| | (SS6V11-E7) VL | CTCCATCTCCTGCAGGTCTAGTCAGAGCCTCCTGCAAAATAATGGATACAACTATTT |
| | NT sequence | GGCTTGGTACCTGCAGAAGCCAGGGCAGTCTCCACAACTCCTCATCTACTTGAGTTC |
| | | TACTCGGGCCTCCGGGGTCCCTGACAGGTTCAGTGGCAGTGGATCAGGCACAGATT |
| | | TTACACTGAAAATCAGCAGAGTGGAGGCTGAGGATGTTGGGGTTTATTACTGCATGC |
| | | AATCTCTACAAATTCCAGGCACCTTCggccaagggacacgactggagattaaa |
| 53 | Antibody 12 | GAGGTGCAGCTGGTGGAGTCTGGGGGAGGCCTGGTCAAGCCTGGGGGGTCCCTGA |
| | (SS6V12-E11) | GACTCTCCTGTGCAGCCTCTGGATTCACCTTCAGTACCTATAACATGAACTGGGTCC |
| | VH NT | GCCAGGCTCCAGGGAAGGGGCTGGAGTGGGTCTCATCCATTAGCAGTAGTAGTAG |
| | sequence | TTTCATGTTCTACGCAGACTCAGTGAAGGGCCGATTCACCATCTCCAGAGACAACGC |
| | | CAATAACTCACTGTATCTGCAAATGAATAGCCTGAGAGCCGAGGACACGGCTGTGTA |
| | | TTATTGTGCGAGAGATTACTATGATAACAGTGGTTACTACTACTACGGTATGGACGT |
| | | CTGGGGCCAAGGGACCACGGTCACCGTCTCTTCA |
| 54 | Antibody 12 | GACATCCAGATGACCCAGTCTCCATCCTCCCTGTCTGCATCTGTAGGCGACAGAGTC |
| | (SS6V12-E11) | ACCATCACTTGCCGGGCAAGTCAGAGCATTAGGACCTATTTAAGTTGGTATCAGCAG |
| | VL NT sequence | AAACCAGGGAAAGCCCCTGAGCTCCTGATCTATGCTGCATCCAATTTGCAAAGTGGG |
| | | GTCCCATCAAGGTTCAGTGGCAGTGGATCTGGGACAGATTTCACTCTCACCATCAGC |
| | | AGTCTGCAACCTGAAGATTTTGCAACTTACTACTGTCTACAGACTTACAGTACCCCTC |
| | | AAATCACCTTCggccaagggacacgactggagattgaa |
| 55 | Antibody 13 | CAGGTGCAGCTGCAGGAGTCGGGCCCAGGACTGGTTAAGCCTTCGGAGACCCTGTC |
| | (SS6V13-F1) | CCTCACCTGCTCTGTCTCTGGTGTCTCCATCCTTGGTTCCTATTGGGGCTGGATCCG |
| | VH NT | GCAGCCCCCCGGGACGGGGCTGCAGTGGATTGGCTATATCTATTTCAGTGAGAACA |
| | sequence | CCAACTACAACCCCTCCCTCCAGAGTCGAGTCACCATGTCACTTGACACGTCCAAGA |
| | | ACCTGTTGTCCCTGAAGCTGAGCTCTGTGACCGCTGCTGACACGGCCGTCTATTACT |
| | | GTGCGAGAGGGGGGGGATATTTGGAAACCGGCCCCTTTGACTCCTGGGGCCAGGG |
| | | AACCCTGGTCACCGTCTCCTCA |
| 56 | Antibody 13 | GATATTGTGATGACTCAGTCTCCACTCTCCCTGCCCGTCACCCCTGGAGAGCCGGC |
| | (SS6V13-F1) VL | CTCCATCTCCTGCAGGTCTAGTCAGGGCCTCGTGCAGAGTAATGGATACAACTATT |
| | NT sequence | TGGCTTGGTACCTGCAGAAGCCAGGGCAGTCTCCACAGCTCCTGATCTATTTGGGTT |
| | | CTAATCGGGCCTCCGGGGTCCCTGACAGGTTCAGTGGCAGTGGATCAGGCACAGAT |
| | | TTTACACTGAAAATCAGCAGAGTGGAGGCTGAGGATGTTGGGGTTTACTACTGCATG |
| | | CAAGGTCTACAAACTCCaggcaccttcggccaagggacacgactggagattaaa |
| 57 | Antibody 14 | CAGGTGCAGCTGCAGGAGTCGGGCCCAGGACTGGTGAAGCCCTCGGAGACCCTGT |
| | (SS6V14-F2) | CCCTCACCTGCACTGTCTCTGGTGGCCCCATCAGTAGTTACTACTGGACCTGGATC |
| | VH NT | CGGCAGCCCCCAGGGAAGGGACTGGAGTGGATTGGATATATCTACTACAGTGGGA |
| | sequence | GCACCAACTACAGCCCCTCCCTCAAGAGTCGAGTCACCATGTCACTAGAAACGTCCA |
| | | AGAACCAGTTCTCCCTGAAGCTGACCTCTGTGACCGCTGCGGACACGGCCGTGTAT |
| | | TACTGTGCGCGAGATCCCCTTGCGGAGGGTGCCGCCTCGTCAGGCTTTGACAACT |
| | | GGGGCCAGGGAACCCTGGTCACCGTCTCCTCA |
| 58 | Antibody 14 | GACATCCAGATGACCCAGTCTCCATCCTCCCTGTCTGCATCCGTCGGAGACAGAGTC |
| | (SS6V14-F2) VL | ACCATCACTTGCCGGGCAAGTCAGAGCATTAGCAGCTATTTAAATTGGTATCAGCAG |
| | NT sequence | AAACCCGGGAAAGCCCCTAAGCTCCTGATCTATGCTGCATCCGCTTTGCAAAGTGGG |
| | | GTCCCATCAAGGTTCAGTGGTAGTGGATCTGGGACACATTTCAGTCTCACCATTAAC |
| | | GGTCTGCAACCTGAAGATTTTGCAACTTACTCCTGTCAACAGAGTTACACTACCCCT |
| | | CGGACTTTCGGCCCTGGGACCAAGGTGGAGATCAAA |
| 59 | Antibody 15 | GAAGTGCAGCTGGTGGAGTCTGGGGGAGGCGTGGTCCAGCCTGGGAGGTCCCTGA |
| | (SS6V15-F6) | GACTCTCCTGTGCAGCCTCTGGATTCACCTTCAGTAGTTATGCTATACACTGGGTCC |
| | VH NT | GCCAGGCTCCAGGCAAGGCGCTGGAGTGGGTGGCACTGATATCATATGATGGACG |
| | sequence | TACAAAATACTACGCAGACTCCGTGAAGGGCCGATTCACCATCTCCAGAGACAATTC |
| | | CAAGAACACCCTGTATCTGCAAATGAACAGCCTGAGAGCTGAGGACACGGCTGTCTA |
| | | TTACTGTGCGAGACTTGATATTATAATTACACCACCTGCTAACGACTACTGGGGCCA |
| | | GGGAACCCTGGTCACCGTCTCTTCA |
| 60 | Antibody 15 | GAAATTGTGTTGACGCAGTCTCCAGGCACCCTGTCTTTGTCTCCAGGGGACAGAGCC |
| | (SS6V15-F6) VL | ACCGTCTCCTGCAGGGCCAGTCAGATTGTTAGCAGCAATTACTTAGCCTGGTACCA |
| | NT sequence | GCAGAAACCTGGCCAGGTTCCCAGACTCCTCATCTATGATGCATCCAGCAGGGCCA |
| | | CTGGCATCCCAGACAGGTTCAGTGGCAGTGGGTCTGGGACAGACTTCACTCTCACC |
| | | ATCAGCAGACTGGAGCCTGAGGATTTTGCAGTGTATTACTGTCACCAGTATGGTGAC |
| | | TCTCGCAGGACGTTCGGCCAAGGGACCAAAGTGGATATCAAA |
| 61 | Antibody 16 | GAGGTGCAGCTGTTGGAGTCTGGGGGAGGCCTGGTCAAGCCGGGGGGGTCCCTGA |
| | (L8N16-C7) VH | GACTCTCCTGTGTAGCCTCTGAATTCACCTTCAGTCGTTATACCATGAACTGGGTCC |
| | NT sequence | GCCAGGCTCCAGGGAAGGGGCTGGAGTGGGTCGCATCCATTGGTGGTAGTACTCC |
| | | TCTCTCATTCTACGCAGACTCATTGAGGGGCCGATTCACCATCTCCAGAGACAACGC |
| | | CAAGAATTCACTCTATCTGGAAATGAGCAGCCTAAGAGCCGACGACACGGCTGTGTA |
| | | TTACTGTGCGCGAGATTCGATTGCCTCGGCGACTACGTTGTTCGATCTCTGGGGCC |
| | | GTGGCACCCCGGTCACTGTCTCCTCA |
| 62 | Antibody 16 | GACATCCAGATGACCCAGTCTCCAGTCTCCCTGTCTGCATCTGTGGGAGACAGAGTC |
| | (L8N16-C7) VL | ACCATCACTTGCCGGGCAAGTCAGGCCATTAGTAGCTATTTAAATTGGTATCAGCAC |
| | NT sequence | AAACCAGGGAAAGCCCCTAGGCTCCTGATCCATGCTGCATCCAGTTTACATACTGGG |
| | | GTCCCATCAAGGTTCAGTGGCAGTGGATCTGGGACAGATTTCACTCTCACCATCAGC |
| | | AGTCTGCAACCTGAAGATTTTGCAACTTACTATTGTCAACAGAGTTACATCACCCCTC |
| | | CGGAGTACAGTTTTGGCCAGGGGACCAAGCTGGAAATGAAA |
| 63 | Antibody 17 | CAGGTGCAGCTGGTGGAGTCTGGGGGAGGCGCGGTCCAGCCTGGGAAGTCCCTGA |
| | (L8N17-G3) VH | GACTCTCCTGTGCAGCCTCTGGCTTCACCTTCAGTTCCTATGCTATGCACTGGGTCC |
| | NT sequence | GCCAGGCTCCAGGCAAGGGGCTAGAGTGGGTGGCAGATATATCATATGATGGACG |
| | | TAATAAATACTACGCAGACTCCGTGAAGGGCCGACTCATCATCTCCAGAGACAATTC |
| | | CAAGAACACGCTGTATCTGCAAATGGACAGCCTGAGACCTGAAGACACGGCTATATA |
| | | TTACTGTGCGAGAGGAGAGGCCGGAACGATGGCTACAATTTGGGTTTCGTCATATG |
| | | ACTACTGGGGCCAGGGATCCCTGGTCACCGTCTCC |
| 64 | Antibody 17 | GATATTGTGATGACCCAGACTCCACTCTCCTCACCTGTCACCCTTGGACAGCCGGCC |
| | (L8N17-G3) VL | TCCATCTCCTGCAGGTCTAGTCAAAGCCTCGTACACAGTGATGGAAACACCTACGT |
| | NT sequence | GAGTTGGCTTCAGCAGAGGCCAGGCCAGCCTCCAAGACTCCTAATTTATAAGATTTC |
| | | TAACCGGTTCTCTGGGGTOCCAGACAGATTCAGTGGCAGTGGGGCAGGGACAGATT |
| | | TCACACTGAAAATCAGCAGGGTGAAAGCTGAGGATGTCGGGGTTTATTTCTGCATGC |
| | | AAGCTACACAATTTCCCCCGACGTTCGGCCAAGGGACCAAGGTGGAAATC |
| 65 | Antibody 18 | GAGGTGCAGCTGGTGGAGTCTGGGGGAGGCTTGGTCCAGCCGGGGGGGTCCCTGA |
| | (SS6V18-E3) | GACTCTCCTGTGCGGCCTCTGGATTCACCTTCAGTTCCTATGCTATGCACTGGGTCC |
| | VH NT | GCCAGGTTCCAGGGAAGGGACTGGAATATGTTTCTTCTATTACTAGTAATGGGGGC |
| | sequence | GGCACATTTTATGCAGACTCTGTGAAGGGCAGATTCACCATCTCCAGAGACAATTCC |
| | | AAGAACACAGTGTATCTGCAAATGGGCAGCCTGAGCACTGAGGACATGGCTTTATAT |
| | | TACTGTGCGAGAGAAGGGATACAGGGATGGGTGACCTACTTTGACTACTGGGGCC |
| | | GGGGAATCCTGGTCACCGTCTCTTCA |
| 66 | Antibody 18 | GACATCCAGATGACCCAGTCTCCATCCTCCCTCTCTGCATCTATAGGAGACAGAGTC |
| | (SS6V18-E3) VL | ACCATCACTTGCCGGGCAAGTCAGAGCATTAGCACCAATTTAAATTGGTATCAGCAA |
| | NT sequence | AAACCAGGGAAAGCCCCCAAGCTCCTGATCTATGCTGCATCCAGTTTGCCAAGTGG |
| | | GGTCCCATCAAGGTTCAGTGGCAGTGGATCTGGGACAGATTTCACTCTCACCATCAG |
| | | CAGTCTGCAACCTGAGGACTTTGCAAATTATCTCTGTCAACAGACTTACACTACGCC |
| | | CCAGTACAGTTTTGGCCAGGGGACCAAGGTGGAGATCAAA |
| 67 | Antibody 19 | GAGGTGCAGCTGGTGCAATCTGGGTCTGAGTTGAAGAAGCCTGGGGCCTCAGTGAA |
| | (SS6V19-F4) | GGTTTCCTGCAAGGTTTCTGGATACACCTTCACTGACTATAATATGAATTGGGTGCG |
| | VH NT | ACAGGCCCCTGGACAAGGGCTTGAGTGGATGGGATGGATCAACACCAACACTGGG |
| | sequence | ATCCCAACTTATGCCCAGGGCTTCACAGGACGGTTTGTCTTCTCCTTGGACACCTCT |
| | | GTCAGCACGGCATATCTGCAGATCAGCAGCCTAAAGGCTGAGGACACTGCCATTTAT |
| | | TACTGTGCGAGAGATGGGGGGTGGCAGTTACCTTACTGGTACTTCGATCTCTGGGG |
| | | CCGTGGCACCCTGGTCACCGTCTCTTCA |
| 68 | Antibody 19 | GAAATAGTGATGACGCAGTCTCCAGCCACCCTGTCTGTGTCTCCAGGGGAAAGAGT |
| | (SS6V19-F4) VL | CACCCTCTCCTGCAGGGCCAGTTACAGTTTTAGCAGCAGCTTAGCCTGGTACCAGC |
| | NT sequence | AGAAACCTGGCCAGGCTCCCAGGCTCCTCATCTACGGTACATCCACCAGGGCCACT |
| | | GGTGTCCCAGTCAGGTTCAGTGGCAGTGGGTCTGGGACAGAGTTCACTCTCACCAT |
| | | CAGCAGCCTCCAGTCTGAAGATTTAGCAGTTTATTACTGTCAACAGTATTATAGTTGG |
| | | CCCCTGACGTTCGGCCAAGGGACCAAAGTGGATATCAAA |
| 69 | Antibody 20 | CAGGTGCAGCTGCAAGAGTCGGGCCCGGGACTGGTGAAGCCTTCGGAGACCCTGT |
| | (SS6V20-F5) | CCCTCACCTGCACTGTCTCTGGTGGCTCCATCATTGGTTACTACTGGAGCTGGATCC |
| | VH NT | GGCAGCCCCCAGGGAAGGGACTGGAGTGGATTGGGTATATCTATTTCAGCGCGAAC |
| | sequence | ACCAACTACAACCCCTCCCTGAAGAGTCGAGTCACCATGTCAGTAGACATGTCCAAG |
| | | AAACAGTTCTCCCTGAAATTGAGGTCTGTGACCGCTGCGGACACGGCCGTTTATTAC |
| | | TGTGCGAGAGGGGGGGGATATTTGGAAACCGGCCCCCTCGACTTCTGGGGCCAGG |
| | | GAATCCTGGTCACCGTCTCCTCA |
| 70 | Antibody 20 | GATATTGTGATGACTCAGTCTCCACTCTCCCTGCCCGTCACCCCTGGAGAGCCGGC |
| | (SS6V20-F5) VL | CTCCATCTCCTGCAGGTCTAGTCAGAGCCTCATGCAGCTTAATGGATACAACTATTT |
| | NT sequence | GGCCTGGTACCTGCAGAGGCCAGGGCAGTCTCCACAGCTCCTGATTTATTTGGGTT |
| | | CTACTCGGGCCTCCGGGGTCCCTGACAGGTTCAGTGGCAGTGGATCAGGCACAGAT |
| | | TTTACACTGAAAATCACCAGAGTGGAGGCTGAGGATGTTGGGGTTTATTATTGCATG |
| | | CAAGGTCTACAAATTCCAGGCACCTTCGGCCAAGGGACACGACTGGAGATTAAA |
| 71 | Antibody 1 | EVQLVESGGGLVQPGGSLRLSCAASGFILRNYEMQWVRQAPGKGLEWVSVIGNTGGTF |
| | (SS6V1-B5) VH | YAGSVKGRFTISRENAKNSLYLQMNSLRAEDTALYYCARVTYTSSPLPFWFLDLWGRGT |
| | aa sequence | LVTVSS |
| 72 | Antibody 1 | DIQMTQSPSSLSASVGDRVTITCRASQSIGNYLNWYQQKSGKAPKILIYAASSLQSGVPS |
| | (SS6V1-B5) VL | RFSGSGSGTDFTLTISSLQPEDFATYYCQQTYRTPPEDSFGQGTKVEIK |
| | aa sequence | |
| 73 | Antibody 2 | EVQLVESGGGLVQPGGSLRLSCAASGFTVSSNYMSWVRQAPGKGLEWVSVIYSGGSTF |
| | (SS6V2-G1) VH | YADSVKGRFTISRDNSKNTLYLQMNSLRAEDTAVYYCARELRHYFDYWGQGTLVTVSS |
| | aa sequence | |
| 74 | Antibody 2 | DIQLTQSPSFLSASVGDRVTITCRASQGISSYLAWYQQKPGKAPKLLIYAASTLQSGVPS |
| | (SS6V2-G1) VL | RFSGSGSGTEFTLTISSLQPEDFATYYCQQLNSYPPYSFGQGTKVEIK |
| | aa sequence | |
| 75 | Antibody 3 | QVQLVQSGAEVKRPGASVKVSCKASGYSFTNSGITWVRQAPGQGLEWMGGTNFYNGI |
| | (SS6V3-G2) VH | TNYPQNIQGRVTMTTDTSTNTAYLELTSLRSEDTAMYYCALNRVAIFNDGYNPLGYWGQ |
| | aa sequence | GTLVTVSS |
| 76 | Antibody 3 | DIVMTQSPDSLAVSLGERATINCKSSQSVLYSSNNKNYLAWYQQKPGQPPKLLIYWASS |
| | (SS6V3-G2) VL | RESGVPDRFSGSGSGTDFTLTIDRLQAEDVAIYYCQQYFSSPFSFGGGTKVEIK |
| | aa sequence | |
| 77 | Antibody 4 | EVQLVQSGAEVKKPGEALKISCQGSGYTFSMYWIAWVRQMPGKGLEWMGIIYPDDSDR |
| | (SS6V4-A1) VH | RYSPSFQGQVTISVDKSISTAYLQWSSLKASDTAIYYCARLQNGYSYGLLENWGQGTLV |
| | aa sequence | TVSS |
| 78 | Antibody 4 | DIVMTQSPDSLTVSLGERATINCKSSQSTLYRSNNKNYLAWYQQKPGQPPKLLINWAST |
| | (SS6V4-A1) VL | REYGVPDRFSGSGSGTDFTLTISSLQAEDVAVYYCQQYYSYPWTFGQGTKVEIK |
| | aa sequence | |
| 79 | Antibody 5 | EVQLVQSGAEVRKPGESLKISCKTSGYTFTHYWIGWVRQLPGKGLEWMGIIYPDDSDTK |
| | (SS6V5-C3) VH | YSPSFQGQITISADKSISTAYLQWNSLKASDTAMYFCATADIVVGSNFFDHWGQGTLVTV |
| | aa sequence | SS |
| 80 | Antibody 5 | DIQMTQSPSTLSASVGDRVTITCRASQSISTWLAWYQQKPGKPPKVLIYKASILESGVSS |
| | (SS6V5-C3) VL | RFSGSGSGTEFTLTISSLQPDDFATYYCQHYNSYIKTFGQGTKVEIK |
| | aa sequence | |
| 81 | Antibody 6 | EVQLVESGGGLVQPGGSLRLSCSASGFTFNTYAMHWVRQAPGKGPEYVSGISSNGGIT |
| | (SS6V6-C4) VH | DYADSVKGRFTISRDNSKNTLYLQMSSLRPEDTAVYYCVKDSLATVVTLLSYWGQGTLV |
| | aa sequence | TVSS |
| 82 | Antibody 6 | DIQMTQSPSSLSASVGDRVTITCRASQTISSYLNWFQQKPGKAPKLLIYAASSLQSGVPS |
| | (SS6V6-C4) VL | RFSGSGSGTDFTLTISSLQPEDFAIYYCQQSYSTPGTFGQGTKVEIK |
| | aa sequence | |
| 83 | Antibody 7 | EVQLVQSGAEVKKPGESLKISCKGSENIFSGYWIGWVRQMPGKGLEWMGIIYPDDSDTR |
| | (SS6V7-C5) VH | YNPSFQGQVTISADQSISTAYLQWSRLKASDTAMYYCARHLGGGSSWPIDYWGQGTLV |
| | aa sequence | TVSS |
| 84 | Antibody 7 | DIQMTQSPSSLSASVGDRVTITCRANQGISNYLAWFQQKPGKAPKSLIYAASSLQSGVPS |
| | (SS6V7-C5) VL | KFSGNRSGTDFTLTISSLQPEDFATYYCQQYSSYPFTFGGGTKVEIK |
| | aa sequence | |
| 85 | Antibody 8 | EVQLVESGGDLVQPGGSLRLSCSASGFTFSTYAMHWVRQAPGKGLDYVASIASDGGIT |
| | (SS6V8-D3) VH | TYADSVKGRFTISRDNSKNALYLQMSSLRVEDTAVYYCVKDSLTSVTTIFDCWGQGTLVT |
| | aa sequence | VSS |
| 86 | Antibody 8 | DIQMTQSPSSLSASVGDSVTITCRASQNINSYLHWYQQKPGKAPKLLIYTASSLQSGVPS |
| | (SS6V8-D3) VL | RFSASGSGTHFTLTISSLQPEDFATYYCQQSYTDPYTFGQGSKVEIK |
| | aa sequence | |
| 87 | Antibody 9 | QVQLQESGPGLVKPSGTLSLTCAVSGGSISSNIWWSWVRQPPGKGLEWIGDIYHSGST |
| | (SS6V9-D11) | NYNPSLEGRVTISIDKSKNQFSLRLTSLTAADTAVYYCARAISQQYFDSSVLGYWGQGIL |
| | VH aa sequence | VTVSS |
| 88 | Antibody 9 | EIVMTQSPATLSVSPGERVTLSCRASQSVVTNLAWYQQKPGQAPRLLIYGASTRATGIPA |
| | (SS6V9-D11) VL | RFSGSGSGTEFTLTISSLQSEDFAIYHCQQYNNWPGYTFGQGTKVDIK |
| | aa sequence | |
| 89 | Antibody 10 | EVQLVQSGAEVKKPGESLKISCQGSEDSFTGYWIGWVRQLPGKGLEWMGFIYPDDGDT |
| | (SS6V10-E1) | RYSPAFQGLVTFSADKSITTAYLQWSSLKASDTAIYFCARHLGGGSSWPIDSWGQGTQV |
| | VH aa sequence | TVSS |
| 90 | Antibody 10 | DIQMTQSPSSLSASVGDRVTITCRASQGIRNYLAWFQQKPGKAPKSLIYAASSLQSGVPS |
| | (SS6V10-E1) VL | KFSGSGYGTDFTLTINSLQPEDFATYYCQQYNNHPFTFGGGTKVEIK |
| | aa sequence | |
| 91 | Antibody 11 | QVQLQESGPGLVKPSETLSLTCTVSGGFIGPHYWSWVRQPPGKGLEWIGYIYISGSTNY |
| | (SS6V11-E7) | NPSLKSRLTISVDMSKSQFSLTLSSATAADTAVYYCARGGGYLETGPFEYWGQGTLVTV |
| | VH aa sequence | SS |
| 92 | Antibody 11 | DIVMTQSPLSLPVTPGEPASISCRSSQSLLQNNGYNYLAWYLQKPGQSPQLLIYLSSTRA |
| | (SS6V11-E7) VL | SGVPDRFSGSGSGTDFTLKISRVEAEDVGVYYCMQSLQIPGTFGQGTRLEIK |
| | aa sequence | |
| 93 | Antibody 12 | EVQLVESGGGLVKPGGSLRLSCAASGFTFSTYNMNWVRQAPGKGLEWVSSISSSSSFM |
| | (SS6V12-E11) | FYADSVKGRFTISRDNANNSLYLQMNSLRAEDTAVYYCARDYYDNSGYYYYGMDVWG |
| | VH aa sequence | QGTTVTVSS |
| 94 | Antibody 12 | DIQMTQSPSSLSASVGDRVTITCRASQSIRTYLSWYQQKPGKAPELLIYAASNLQSGVPS |
| | (SS6V12-E11) | RFSGSGSGTDFTLTISSLQPEDFATYYCLQTYSTPQITFGQGTRLEIE |
| | VL aa sequence | |
| 95 | Antibody 13 | QVQLQESGPGLVKPSETLSLTCSVSGVSILGSYWGWIRQPPGTGLQWIGYIYFSENTNY |
| | (SS6V13-F1) | NPSLQSRVTMSLDTSKNLLSLKLSSVTAADTAVYYCARGGGYLETGPFDSWGQGTLVTV |
| | VH aa sequence | SS |
| 96 | Antibody 13 | DIVMTQSPLSLPVTPGEPASISCRSSQGLVQSNGYNYLAWYLQKPGQSPQLLIYLGSNR |
| | (SS6V13-F1) VL | ASGVPDRFSGSGSGTDFTLKISRVEAEDVGVYYCMQGLQTPGTFGQGTRLEIK |
| | aa sequence | |
| 97 | Antibody 14 | QVQLQESGPGLVKPSETLSLTCTVSGGPISSYYWTWIRQPPGKGLEWIGYIYYSGSTNY |
| | (SS6V14-F2) | SPSLKSRVTMSLETSKNQFSLKLTSVTAADTAVYYCARDPLAEGAASSGFDNWGQGTL |
| | VH aa sequence | VTVSS |
| 98 | Antibody 14 | DIQMTQSPSSLSASVGDRVTITCRASQSISSYLNWYQQKPGKAPKLLIYAASALQSGVPS |
| | (SS6V14-F2) VL | RFSGSGSGTHFSLTINGLQPEDFATYSCQQSYTTPRTFGPGTKVEIK |
| | aa sequence | |
| 99 | Antibody 15 | EVQLVESGGGVVQPGRSLRLSCAASGFTFSSYAIHWVRQAPGKALEWVALISYDGRTK |
| | (SS6V15-F6) | YYADSVKGRFTISRDNSKNTLYLQMNSLRAEDTAVYYCARLDIIITPPANDYWGQGTLVT |
| | VH aa sequence | VSS |
| 100 | Antibody 15 | EIVLTQSPGTLSLSPGDRATVSCRASQIVSSNYLAWYQQKPGQVPRLLIYDASSRATGIP |
| | (SS6V15-F6) VL | DRFSGSGSGTDFTLTISRLEPEDFAVYYCHQYGDSRRTFGQGTKVDIK |
| | aa sequence | |
| 101 | Antibody 16 | EVQLLESGGGLVKPGGSLRLSCVASEFTFSRYTMNWVRQAPGKGLEWVASIGGSTPLS |
| | (L8N16-C7) VH | FYADSLRGRFTISRDNAKNSLYLEMSSLRADDTAVYYCARDSIASATTLFDLWGRGTPV |
| | aa sequence | TVSS |
| 102 | Antibody 16 | DIQMTQSPVSLSASVGDRVTITCRASQAISSYLNWYQHKPGKAPRLLIHAASSLHTGVPS |
| | (L8N16-C7) VL | RFSGSGSGTDFTLTISSLQPEDFATYYCQQSYITPPEYSFGQGTKLEMK |
| | aa sequence | |
| 103 | Antibody 17 | QVQLVESGGGAVQPGKSLRLSCAASGFTFSSYAMHWVRQAPGKGLEWVADISYDGRN |
| | (L8N17-G3) VH | KYYADSVKGRLIISRDNSKNTLYLQMDSLRPEDTAIYYCARGEAGTMATIWVSSYDYWG |
| | aa sequence | QGSLVTVS |
| 104 | Antibody 17 | DIVMTQTPLSSPVTLGQPASISCRSSQSLVHSDGNTYVSWLQQRPGQPPRLLIYKISNRF |
| | (L8N17-G3) VL | SGVPDRFSGSGAGTDFTLKISRVKAEDVGVYFCMQATQFPPTFGQGTKVEI |
| | aa sequence | |
| 105 | Antibody 18 | EVQLVESGGGLVQPGGSLRLSCAASGFTFSSYAMHWVRQVPGKGLEYVSSITSNGGGT |
| | (SS6V18-E3) | FYADSVKGRFTISRDNSKNTVYLQMGSLSTEDMALYYCAREGIQGWVTYFDYWGRGILV |
| | VH aa sequence | TVSS |
| 106 | Antibody 18 | DIQMTQSPSSLSASIGDRVTITCRASQSISTNLNWYQQKPGKAPKLLIYAASSLPSGVPSR |
| | (SS6V18-E3) VL | FSGSGSGTDFTLTISSLQPEDFANYLCQQTYTTPQYSFGQGTKVEIK |
| | aa sequence | |
| 107 | Antibody 19 | EVQLVQSGSELKKPGASVKVSCKVSGYTFTDYNMNWVRQAPGQGLEWMGWINTNTGI |
| | (SS6V19-F4) | PTYAQGFTGRFVFSLDTSVSTAYLQISSLKAEDTAIYYCARDGGWQLPYWYFDLWGRGT |
| | VH aa sequence | LVTVSS |
| 108 | Antibody 19 | EIVMTQSPATLSVSPGERVTLSCRASYSFSSSLAWYQQKPGQAPRLLIYGTSTRATGVPV |
| | (SS6V19-F4) VL | RFSGSGSGTEFTLTISSLQSEDLAVYYCQQYYSWPLTFGQGTKVDIK |
| | aa sequence | |
| 109 | Antibody 20 | QVQLQESGPGLVKPSETLSLTCTVSGGSIIGYYWSWIRQPPGKGLEWIGYIYFSANTNYN |
| | (SS6V20-F5) | PSLKSRVTMSVDMSKKQFSLKLRSVTAADTAVYYCARGGGYLETGPLDFWGQGILVTV |
| | VH aa sequence | SS |
| 110 | Antibody 20 | DIVMTQSPLSLPVTPGEPASISCRSSQSLMQLNGYNYLAWYLQRPGQSPQLLIYLGSTR |
| | (SS6V20-F5) VL | ASGVPDRFSGSGSGTDFTLKITRVEAEDVGVYYCMQGLQIPGTFGQGTRLEIK |
| | aa sequence | |
| 111 | Heavy chain | ESTPXTXG |
| | variable region | |
| | HC-CDR1 | |
| 112 | Heavy chain | IVXSVAN |
| | variable region | |
| | HC-CDR2 | |
| 113 | Heavy chain | DXDTXTTTXXHSXSPVP |
| | variable region | |
| | HC-CDR3 | |
| 114 | light chain | RTIATX |
| | variable region | |
| | LC-CDR1 | |
| 115 | light chain | ADS |
| | variable region | |
| | LC-CDR2 | |
| 116 | light chain | QRSXXTXRGAX |
| | variable region | |
| | LC-CDR3 | |

In sequence ID Nos. 31-70 listed above the nucleic acids encoding the CDR regions are bold and underlined. In sequence ID Nos. 71-110 listed above the amino acids of the CDR regions are bold and underlined.

### EXAMPLES

In the following, the inventors present by way of example only, antigen-binding molecule which binds to a wide range of sarbecovirus spike protein.

### Example 1 Human Serum Panels

The four serum panels included in this study were described as below. (1) SARS patient (n=11): these were sera collected from SARS survivors in Singapore at different time points (2003, 2012 and 2020) before the vaccination program started in February 2021; (2) COVID-19 patient (n=40): this group of sera was collected during 2020 as part of a national longitudinal study (Chia et al.: Lancet Microbe 2021). (3) Healthy individuals-vaccinated with COVID-19 vaccine (in this case the Pfizer mRNA vaccine) (n=20): these were sera collected at day 14 after second dose (or 35 days after the first dose) of the Pfizer-BioNTech BNT162b2 mRNA vaccine. (4) SARS survivors-vaccinated with COVID-19 vaccine (in this case the Pfizer mRNA vaccine) (n=9): sera taken from SARS survivors 21-62 days post first vaccination of the Pfizer-BioNTech BNT162b2 mRNA vaccine.

To our surprise, when SARS survivors were immunized with COVID-19 vaccine (in this case the Pfizer mRNA vaccine), we found an unexpected high-level boost of anti-SARS-COV NAbs (See [Table 1]).

TABLE 1
Boost of SARS-CoV NAb after immunization with COVID-19
vaccine (NT50%).

| SARS Survivors | | Patient #1 | Patient #2 |
|---|---|---|---|
| SARS NAbs | Pre-vaccine | 170 | 266 |
| | Post-vaccine | 1,077 | 1,266 |
| | Fold of change | 6.34 | 4.76 |
| COVID NAbs | Post-vaccine | 719 | 1,115 |

Although some level of boosting was deemed possible considering that the SARS-COV and SARS-COV-2 share genome identity at approximately 80% (Zhou et al.: Nature 2020, 579:270-273.), the fold of increase (5-6 fold) SARS-COV specific NAbs was not expected. Importantly, this observation was not limited to the first two SARS survivors tested, the trend maintained (see below) when more individuals (N=9) were tested.

### Multiplex Surrogate Virus Neutralizing Test (SVNT) Based on RBD From Six Different Sarbecoviruses

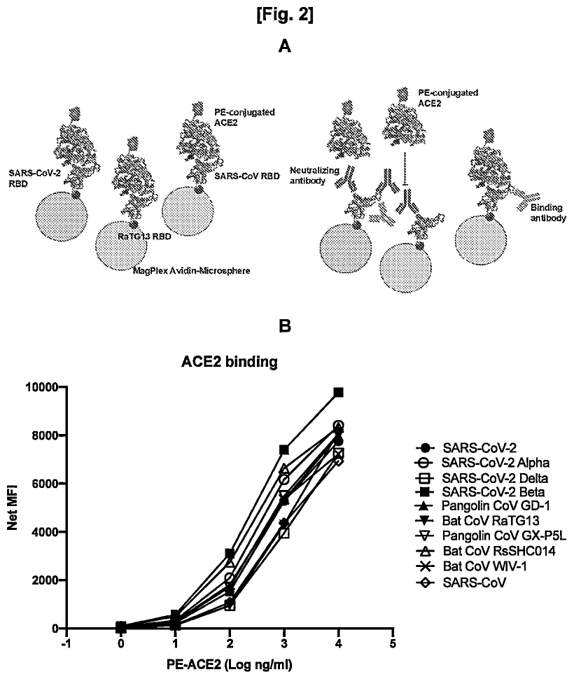

The viral RBD were immobilized on a solid phase (a magnetic bead) and used with a fluorescent dye, in this case phycoerythrin (PE), conjugated to ACE2 to measure the virus-receptor binding ([FIG. 2]) which allowed multiplex detection of NAbs against different sarbecoviruses. Altogether six RBD proteins from six different sarbecoviruses were used SARS-COV; SARS-COV-2; SARS-COV-2 B.1.1.7; SARS-COV-2 B1.351; SC2r-COV GX-P5L and SC2r-COV RaTG13.

Six AviTag-biotinylated RBDs from different sarbecoviruses were coated on the MagPlex Avidin microsphere (Luminex) at 5 μg/1 million beads. In multiplex sVNT, RBD-coated microspheres (600 beads/antigen) were pre-incubated with serum at a final concentration of 1:20 or greater for 1 h at 37° C. with 800 rpm agitation. After 1 h incubation, 50 μl of PE-conjugated hACE2 (GenScript, 1000 ng/ml) were added to the well and incubated for 30 min at 37° C. with agitation, followed by two PBS-1% BSA washes. The data were acquired using MAGPIX system.

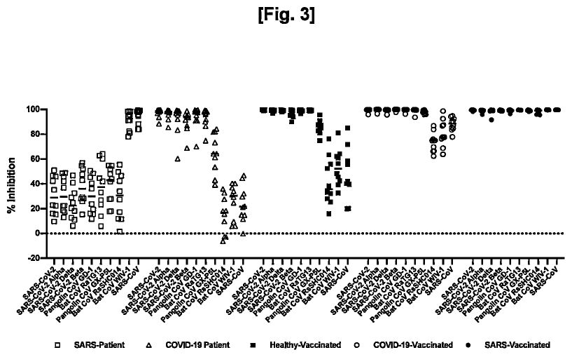

The cross-NAb data demonstrated two highly important features: 1) vaccination of SARS survivors produced very high NAbs against all viruses studied ([FIG. 3D]), even against the bat and pangolin viruses; 2) they neutralized the SARS-COV-2 variants better than the naïve individuals who received the normal two doses ([FIG. 3C]); 3) SARS patients had minimal cross-NAbs to any of the other five viruses before vaccination ([FIG. 3A]) whereas the COVID-19 patients had cross-NAbs against other viruses (all of them are SARS-COV-2 related), they did not have much against the SARS-COV ([FIG. 3B]).

The mRNA vaccine has been demonstrated to have an exceptional capability of inducing very high level of neutralizing antibodies (Nabs) against SARS-COV-2. But from the breakthrough infections reported (Hacisuleyman et al.: N Engl J Med 2021) and the data in [FIG. 3C], it is clear that the NAbs have a relatively narrow spectrum as some individuals had lower NAbs against the Alpha COVID-19 variant SARS-COV-2 B.1.1.7; and/or the Beta COVID-19 variant SARS-COV-2 B.1.351 also known as 20H/501Y.V2, or 501Y.V2 variant; i.e., the NAbs are highly specific to the virus sequence used in the mRNA vaccine. The same was observed with the COVID-19 patient sera ([FIG. 3B]).

Although SARS-COV and SARS-COV-2 share 80% genome identify and cross-NAbs have been discovered in the past (both in human and animals), majority of the key immune dominant neutralizing epitopes in the RBD region of their spike protein (S) are highly virus- or variant-specific. However unexpectedly, when cross immunization is conducted (either through infection or vaccination), it is possible to make the cross-NAb epitope(s) more immune dominant.

### Pan-Sarbecovirus mAb Inhibition Assay

Using the multiplex sVNT developed above, the study was extended to examine cross-NAbs to the SARS-COV-2 variants and, more importantly, to other sarbecoviruses that are detected in bats and pangolins which are deemed to be potentially at risk for human infection (Lam et al.: Nature 2020, 583:282-285.).

RBD-coated microspheres (600 beads/antigen) were pre-incubated with 1:100 diluted serum for 1 h at 37° C. with agitation. Unbound antibodies were removed by two PBS-1% BSA washes. Pan-sarbecovirus mAbs (1000 ng/ml) were then added, followed by 1 h incubation at 37° C. with agitation followed by washing. The binding of the pan-sarbecovirus mAb on RBD was detected by PE-conjugated anti-mouse IgG antibodies. The data were acquired using MAGPIX system.

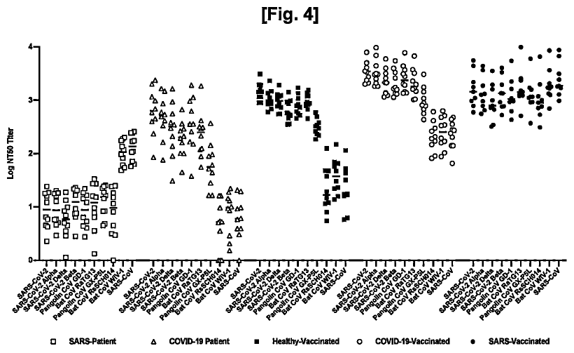

Using serial dilutions, we have further illustrated the best performance of pan-sarbecovirus cross-neutralization by the SARS-vaccinated group ([FIG. 4]). This is the first real life human study/data demonstrating the feasibility of a pan-sarbecovirus antigen-binding molecule.

While previous studies, had shown there is limited cross neutralization between SARS-COV and SARS-COV-2 (Yang R, et al. EBioMedicine 2020, 58:102890), when SARS survivors were immunized with COVID-19 vaccine (in this case the Pfizer mRNA vaccine), we found that a high level of cross-NAbs were produced which can neutralize six different sarbecoviruses used in the current study ([FIG. 4]). This is the first-in-human study/data demonstrating the feasibility of producing pan-sarbecovirus NAbs with high potency and broad spectrum.

The mRNA vaccine has been demonstrated to have an exceptional capability of inducing very high level of NAbs against SARS-COV-2. But from the breakthrough infections reported (Hacisuleyman, et al. N Engl J Med 2021.) and the data in [FIG. 4], it is clear that the NAbs have a relatively narrow spectrum as some individuals had lower NAbs against the Alpha COVID-19 variant SARS-COV-2 B.1.1.7; and/or the Beta COVID-19 variant SARS-CoV-2 B.1.351 also known as 20H/501Y.V2, or 501Y.V2 variant; and even lower NAbs against the pre-emergent bat and pangolin viruses, with the NAs against SARS-COV-1 at the lowest.

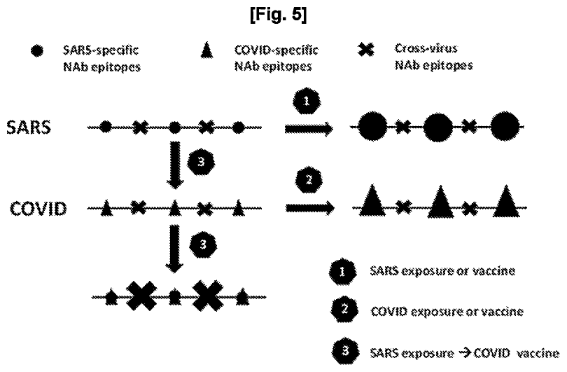

As depicted in [FIG. 5] it is postulated that the most likely mechanism of this cross-neutralization boosting is via exposure to two separate epitopes from two different sarbecoviruses: although SARS-COV-1 and SARS-COV-2 share 80% genome identify and cross-NAbs have been discovered in the past (both in human and animals), majority of the key immune dominant neutralizing epitopes in the RBD region of their spike protein (S) are highly virus- or variant-specific. However, when cross immunization is conducted (either through infection or vaccination), it is possible to make the cross-NAb epitope(s) more immune dominant.

SARS-COV-1 survivors who received the BNT162b2 mRNA vaccine generated broad neutralizing antibodies against ten sarbecoviruses in clades 1a and 1b, including multiple VOCs of SARS-COV-2 and zoonotic sarbecoviruses.

### Example 2. Mouse Studies

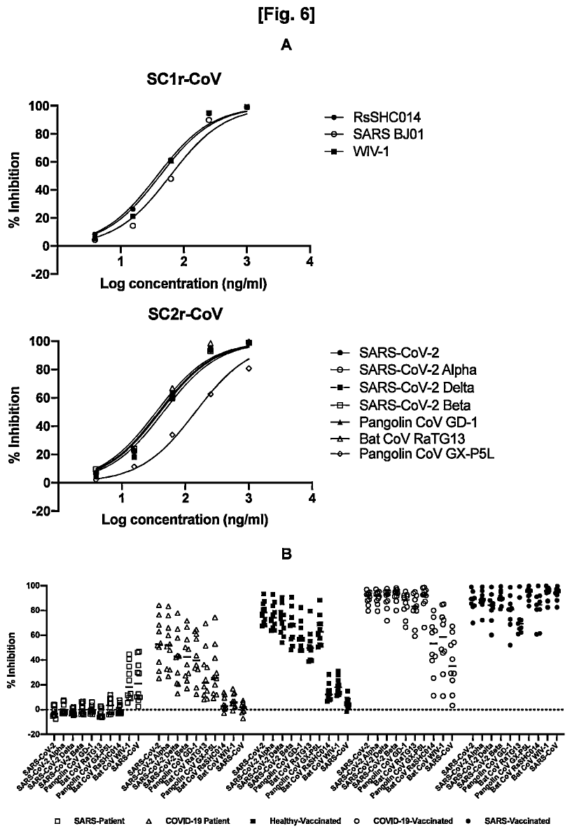

In this study, the mouse mAb 5B7D7 (Genscript) was used. As shown in [FIG. 6A], the mAb is able to neutralize all six viruses, albeit with slightly lower efficacy against GX-P5L. Using a blocking assay, same principle as the sVNT by replacing the PE-hACE2 with the mAb, we have determined the ability of the four different serum panels to block the mAb's ability in neutralization ([FIG. 6B]). Several important findings were made from this analysis. First, it is clear that the cross-neutralizing ability of the SARS-Vaccinated group is the best among the four groups. Second, during natural infection (for either SARS and COVID-19), there is minimal activation of cross-neutralizing antibodies across the two lineages between SARS-COV-2 (and related viruses) and SARS-COV-1. Third, although the mRNA vaccination boosted overall neutralizing ability against SARS-COV-2 related viruses, it had minimal impact on cross-neutralization against SARS-COV-1.

Although cross neutralization between SARS-COV-1 and SARS-COV-2 is not common, this monoclonal antibody binds to RBD and cross-neutralize both SARS-COV-1 and SARS-COV-2 as well as other sarbecoviruses.

### B-Cell Profiling by Staining With RBD From SARS-COV-1 and SARS-COV-2

For flow cytometry analysis, cryopreserved PBMCs were thawed and surface stained for SARS-COV-1 and SARS-COV-2 specific B cells using bait tetramers prepared with biotinylated SARS-COV-1 RBD or SARS-COV-2 RBD (custom-made by GenScript) tetramerised with Streptavidin-conjugated with BV421 (Biolegend, Cat #405225) or Streptavidin-conjugated with PE (BD Pharmigen, Cat #554061). Briefly, thawed PBMCs were incubated for 40 min at room temperature with SARS-COV-1-RBD tetramers and SARS-COV-2-RBD tetramers with 10% FBS in FACS staining buffer (PBS supplemented with 2 mM EDTA and 2% FBS) before proceeding to staining with surface panel fluorochrome-conjugated antibodies. Surface staining was performed with viability dye (Invitrogen, LIVE/DEAD® Fixable AQUA Dead Cell Stain), anti-human CD3 antibody conjugated with

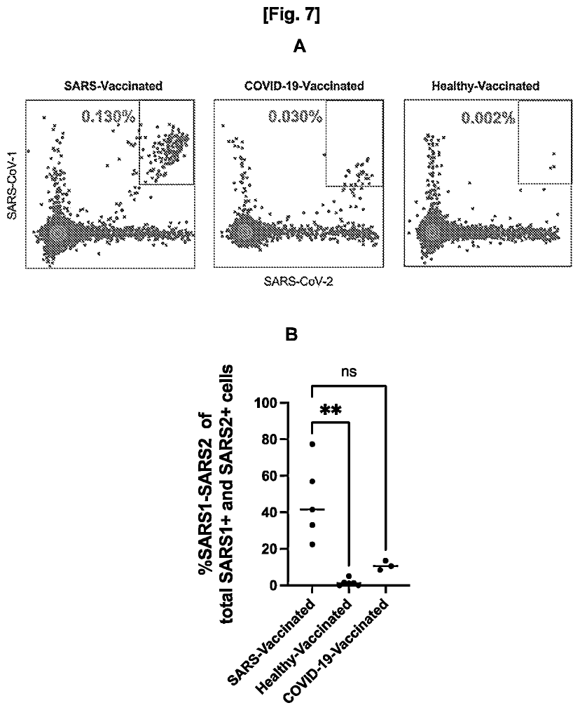

FITC, anti-human CD14 antibody conjugated with FITC, anti-human CD56 antibody conjugated with FITC, anti-human CD19 antibody conjugated with PE-Cyanine5, anti-human CD27 antibody conjugated with APC-H7 and anti-human CD38 antibody conjugated with BV786, for 30 min in FACS staining buffer at 4° C. Stained cells were washed twice with FACS staining buffer and acquired on the same day. Samples were acquired on BD LSR Fortessa™ analyser or BD FACS Aria III equipped with 355, 405, 488, 561, and 640 nm lasers. SARS-COV-1 and SARS-COV-2 specific B cells were quantified by gating on CD19+ B cells after excluding AQUA-positive dead cells and CD3+, CD14+, CD56+ cells. The boosting of cross-NAbs in the SARS-Vaccinated group was further confirmed by direct staining of B-cells using virus-specific RBD proteins. As shown in [FIG. 7], the double stained B-cells, i.e., B-cells which bind RBDs from both SARS-COV-1 and SARS-COV-2, were significantly enriched, by more than 10-fold, in the SARS-Vaccinated group in comparison to the Healthy-Vaccinated group.

### Example 3 Rabbit Studies

The virus/strain-specific immunodominant antibody responses were further confirmed using rabbit hyper immune serum targeting specific virus/strain. In addition to the four key viruses used in this study (i.e., SARS-COV, RaTG13, GX-P5L and SARS-COV-2), we have also included RmYN02 and HKU1. RmYN02 is a bat sarbecovirus which has a very close genetic relationship with SARS-COV-2, but its RBD failed to bind hACE2 (Wacharapluesadee et al.: Nat Commun 2021, 12:972). RmYN02 is also very closely related to another bat sarbecovirus, RacCS203, discovered in bats in Thailand (Wacharapluesadee et al.: Nat Commun 2021, 12:972). HKU1 is a human beta coronavirus, but not a sarbecovirus, and is included here as a negative control.

### Multiplex sVNT Analysis Using Rabbit Hyper Immune Sera Targeting RBD of Six Different Beta Coronaviruses

The rabbit anti-RBD sera were produced by commercial contract with GenScript Biotech using the RBD of each virus as the antigen. The testing was conducted essentially the same as those described above. Rabbit sera were used by a 4-fold serial dilution starting at 1:20.

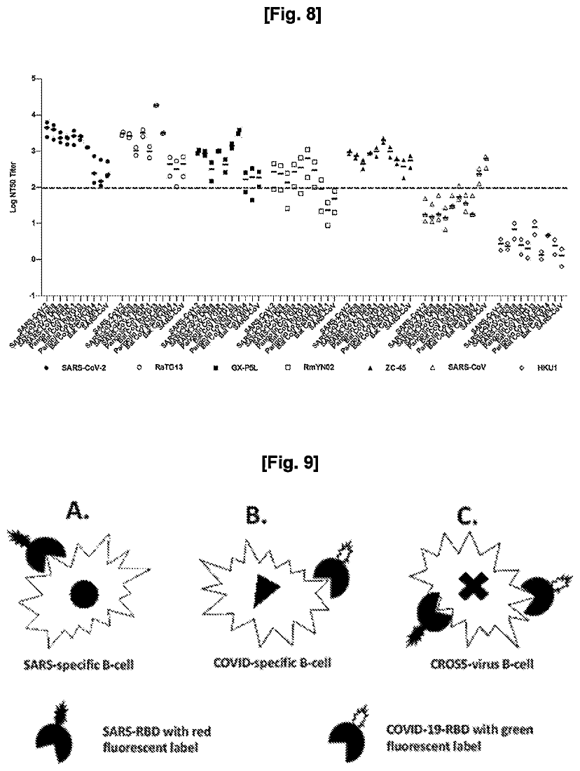

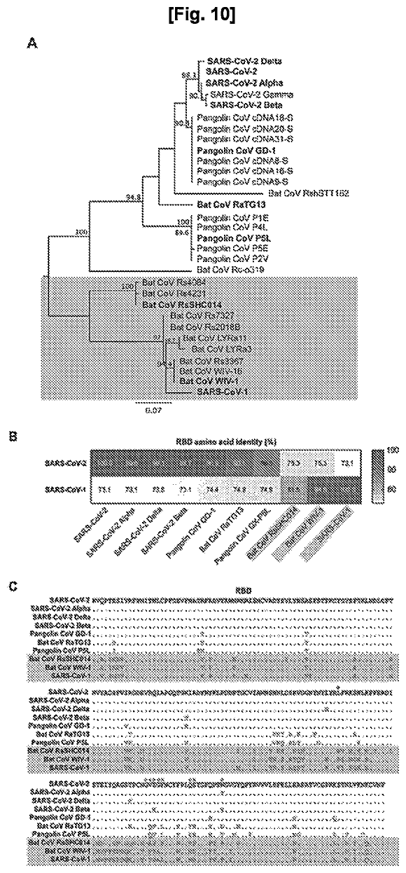

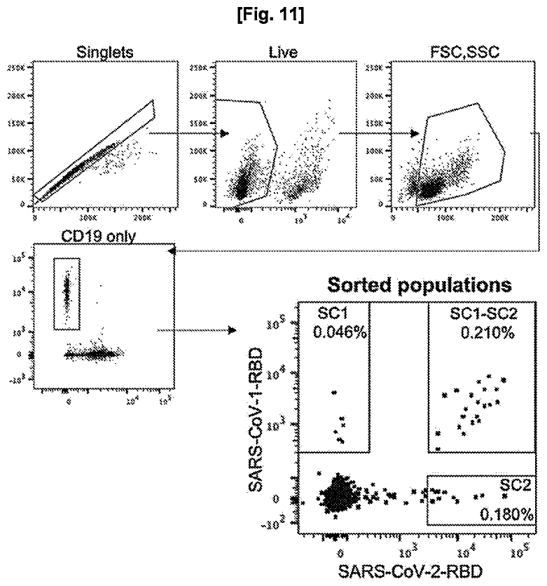

The data presented in [FIG. 8] demonstrates that cross-neutralization is limited only to the strain/lineage level among the five Sarbecoviruses SC2r-CoVs. There is no cross-neutralization between SC2r-COV and SARS-COV and the negative control HKU1 did not neutralize any virus/strain as shown. It is worth noting that the RmYN02, although shown to be non-hACE2 binding (Wacharapluesadee et al.: Nat Commun 2021, 12:972), is still able to produce neutralizing antibodies to the bat sarbecovirus RaTG13.

Based on various embodiments described above from the SARS-Vaccinated donors, human pan-sarbecovirus NAbs are formed as follows:

Sorting out SARS1-SARS2 double positive B-cells: as shown in [FIG. 9], using RBDs from SRARS-COV-1 and SARS-COV-2, B-cells which produce antibodies that can recognize both RBDs (type C in the figure) are selected and tested for neutralization across different sarbecoviruses.

Cloning and culture B-cells for production of small scale monoclonal antibodies: two methods are used for detecting the B-cell receptors. First, the single B cells are grown on 3T3 feeder system which will allow continual secretion of mAbs into the supernatant for initial screening, followed by B cell receptor cloning of the best clones. Second, the sorted B are lysed directly to cells and B cell receptors sequences are detected from the RNA followed by subcloning the sequences into an mAb expression plasmid.

Testing for pan-sarbecovirus neutralization activity: supernatant containing individual mAb are used to test for cross-NAb activity using the multiplex sVNT platform described herein above.

Large scale production and further characterization: top candidates are taken for further characterization such as structural analysis for epitope mapping, determination of neutralization activity against live virus and checking in-vivo protection in animal challenge models.

### Example 4. Isolation of Broadly Sarbecovirus-Neutralizing mAbs From a BNT162b2-Vaccinated SARS1 Survivor Donor

Blood was obtained from a SARS-COV-1 survivor, SS6V, and the neutralization capacity of the donor's sera to SARS-COV-1 and SARS-COV-2 was confirmed pre- and post-BNT162b2 vaccination using a surrogate virus neutralization test. PBMCs and plasma from day 23 post first vaccination dose were separated from EDTA whole blood and cryopreserved for long term storage. PBMCs were thawed, first stained for SARS-COV-1 RBD and SARS-COV-2 RBD tetramers, then surface stained with LIVE/DEAD Fixable aqua dead cell strain (Invitrogen), anti-human CD3-FITC, anti-human CD14-FITC, anti-human CD56-FITC, anti-human CD19-PE-Cy5, anti-human CD27-APC-H7, anti-human CD38-BV786 were single cell sorted into 96-well PCR plates (Axygen) pre-filled with 10 μl/well of RT-PCR catch buffer containing 10 mM TRIS pH 8.0 and 10 U of RNasin Ribonuclease Inhibitor (Promega) using BD FACSAria III (BD Biosciences) equipped with 355-nm, 405-nm, 488-nm, 561-nm and 640-nm lasers. The plates were then flash frozen on dry ice and kept at −80° C. until use.

Reverse transcription was next performed using Qiagen OneStep RT-PCR kit on each plate of sorted B cells. Nested PCR was done using Q5 polymerase (NEB) and the wells containing corresponding heavy and light chains were purified for sequencing. After analyzing the sequences, specific primers were then used to amplify for individual gene families and the PCR products were cloned into pTRIOZ expression vector (Invivogen).

pTRIOZ constructs were transfection into HEK293 cells using Fugene6 (Promega) and the supernatant was harvested to check for small scale efficacy screening using SARS-COV-1 and SARS-COV-2 RBD binding ELISA and surrogate virus neutralization tests (SVNT). For the binding ELISA assay, 100 ng of protein was coated onto Maxisorp plates (Nunc) overnight at 4° C. After blocking with OptEIA blocking buffer (BD), 50 μl of each supernatant were added neat per well and incubated at 37° C. for 1 h. 1:5000 of Goat anti-human IgG-HRP (Bethyl) diluted in OptEIA was then added which will convert TMB substrate into a colorimetric readout for quantification using Cytation 5 reader (BioTek). For SVNT, a commercially available kit for SARS-COV-2 (cPass; Genscript) was used and the manufacturer's protocol was followed. The same kit was used to measure the amount of neutralizing antibodies against SARS-COV-1 by substituting the SARS-COV-2 RBD-HRP reagent with 6ng/well of SARS-COV-1 RBD-HRP (Genscript). The best mAbs (SS6V1-B5, SS6V11-E7, SS6V12-E11, SS6V13-F1, SS6V19-F4 and SS6V20-F5) and control mAbs (S309, CR3022, S2X259 and LyCoV-1404) from the initial binding and neutralization screening were then batch produced by transfection into EXPI293 cells for large-scale expression and purified using Protein G agarose beads (Millipore) for downstream characterizations.

The results are summarized in [Table 2]

TABLE 2
Pre-and post-vaccination neutralizing antibody levels of the donor recruited
in this study measured by sVNT

| | | |
|---|---|---|
| Pre-vaccination | Post dose 1 | Post dose 2 |

SARS-CoV-1 sVNT (% inhibition)

| | | |
|---|---|---|
| 73.05 | 98.12 | 97.74 |

SARS-CoV-2 sVNT (% inhibition)

| | | |
|---|---|---|
| 37.31 | 97.72 | 97.06 |

The peripheral blood mononuclear (PBMCs) at day 23 after the first dose of BNT162b2 vaccine were taken for B cell enrichment and isolation. CD19+ B cells that were positive for binding to SARS-COV-1 (SC1+) and SARS-COV-2 (SC2+) RBD tetramers [FIG. 11] were sorted and their antibody genes amplified and cloned. In total, 19 paired heavy and light kappa fragments were recovered from single B cells, reconstructed into pTRIOZ expression vector and monoclonal antibodies (mAbs) were expressed in vitro. Majority (17 out of 19) were from the double-positive (SC1+SC2+) B cells, while two from SC2+ and none from the SC1+ single-positive B cells were recovered.

For control and comparative studies, we also included four published broad spectrum mAbs, S309 (Sotrovimab, GSK), CR3022, S2X259 (VirBiotech) and LyCoV-1404 (Eli Lily) in this study. The two mAbs cloned from SC2+ single positive B cells showed minimal reactivity to SARS-COV-1 RBD [Table 3]. All 17 mAbs recovered from SC1+SC2+ B cells showed binding to both SARS-COV-1 and SARS-COV-2 RBDs, but their neutralization capacities varied across different mAbs. Six of the most potent neutralizers (SS6V1-B5, SS6V11-E7, SS6V12-E11, SS6V13-F1, SS6V19-F4 and SS6V20-F5) were selected for large-scale production and further characterization. These are listed as antibodies 1, 11-13, 19 and 20 above. The most potent mAbs (SS6V11-E7, SS6V13-F1 and SS6V20-F5) utilize a unique heavy and light chain gene family combination that has not been reported to date. These are listed as antibodies 11, 13 and 20 above. All three mAbs use IGHV4-59 heavy chains and IGKV2-28/IGKJ5 light chains, suggesting that the B cells most likely originated from the same clonal family and the slight variations of the heavy and light chains occurred through hypersomatic mutation.

TABLE 3
Binding and neutralization activities of the mAbs obtained in this study

| | | | | sVNT | sVNT | | |
|---|---|---|---|---|---|---|---|
| | | | | SARS- | SARS- | Binding | Binding |
| | | Construct | | CoV-1 | CoV-2 | SARS- | SARS- |
| | Type | name | Gene class | (% I) | (% I) | CoV-1 | CoV-2 |
| 1 | SC2+ | mAb2 | H = 3-66, K = 1-9 | 1.346 | 20.159 | 0.008 | 0.947 |
| | | (SS6V2-G1) | | | | | |
| 2 | SC2+ | mAb3 | H = 1-18, K = 4-1 | 8.882 | 17.704 | 0.010 | 1.088 |
| | | (SS6V3-G2) | | | | | |
| 3 | SC1+SC2+ | mAb4 | H = 5-51, K = 4-1 | 15.311 | 22.372 | 0.960 | 1.251 |
| | | (SS6V4-A1) | | | | | |
| 4 | SC1+SC2+ | mAb1 | H = 3-13, K = 1-39 | 52.063 | 46.266 | 1.346 | 1.731 |
| | | (SS6V1-B5) | | | | | |
| 5 | SC1+SC2+ | mAb5 | H = 5-51, K = 1-5 | 11.693 | 32.296 | 1.159 | 1.677 |
| | | (SS6V5-C3) | | | | | |
| 6 | SC1+SC2+ | mAb6 | H = 3-64, K = 1-39 | 30.472 | 31.708 | 1.293 | 1.917 |
| | | (SS6V6-C4) | | | | | |
| 7 | SC1+SC2+ | mAb7 | H = 5-51, K = 1-16 | 20.813 | 14.903 | 0.972 | 1.164 |
| | | (SS6V7-C5) | | | | | |
| 8 | SC1+SC2+ | mAb8 | H = 3-64, K = 1-39 | 36.094 | 33.057 | 1.417 | 1.938 |
| | | (SS6V8-D3) | | | | | |
| 10 | SC1+SC2+ | mAb9 | H = 4-4, K = 3-15 | 45.215 | 39.177 | 1.295 | 1.815 |
| | | (SS6V9-D11) | | | | | |
| 11 | SC1+SC2+ | mAb10 | H = 5-51, K = 1-16 | 15.401 | 22.683 | 1.096 | 1.308 |
| | | (SS6V10-E1) | | | | | |
| 12 | SC1+SC2+ | mAb18 | H = 3-64, K =1-39 | 43.660 | 15.526 | 1.329 | 1.109 |
| | | (SS6V18-E3) | | | | | |
| 13 | SC1+SC2+ | mAb11 | H = 4-59, K = 2-28 | 96.890 | 96.196 | 1.133 | 1.676 |
| | | (SS6V11-E7) | | | | | |
| 14 | SC1+SC2+ | mAb12 | H = 3-21, K = 1-39 | 50.389 | 45.021 | 1.213 | 1.445 |
| | | (SS6V12-E11) | | | | | |
| 15 | SC1+SC2+ | mAb13 | H = 4-59, K = 2-28 | 95.903 | 94.537 | 1.248 | 1.694 |
| | | (SS6V13-F1) | | | | | |
| 16 | SC1+SC2+ | mAb14 | H = 4-59, K = 1-39 | 18.122 | 33.402 | 1.062 | 1.539 |
| | | (SS6V14-F2) | | | | | |
| 17 | SC1+SC2+ | mAb19 | H = 7-4-1, K = 3-15 | 61.214 | 38.797 | 1.149 | 0.641 |
| | | (SS6V19-F4) | | | | | |
| 18 | SC1+SC2+ | mAb20 | H = 4-59, K = 2-28 | 96.651 | 96.058 | 1.246 | 1.805 |
| | | (SS6V20-F5) | | | | | |
| 19 | SC1+SC2+ | mAb15 | H = 3-30, K = 3-20 | 17.494 | 15.664 | 0.831 | 0.674 |
| | | (SS6V15-F6) | | | | | |
| 20 | Control | S309 | H = 1-18, K = 3-20 | 36.513 | 39.592 | 1.216 | 1.546 |
| 21 | Control | CR3022 | H = 5-51, K = 4-1 | 32.536 | 24.205 | 1.185 | 1.463 |
| 22 | Control | S2X259 | H = 1-69, L = 1-40 | 92.793 | 94.018 | 0.973 | 1.731 |
| 23 | Control | LyCoV1404 | H = 2-5, L = 2-14 | 94.522 | 0.144 | 1.544 | 0.445 |

(H = heavy gene family, K = kappa light gene family, L = lambda light gene family)

### Example 5 Potency, Breadth and Variant Escapability of Pan-Sarbecovirus mAbs

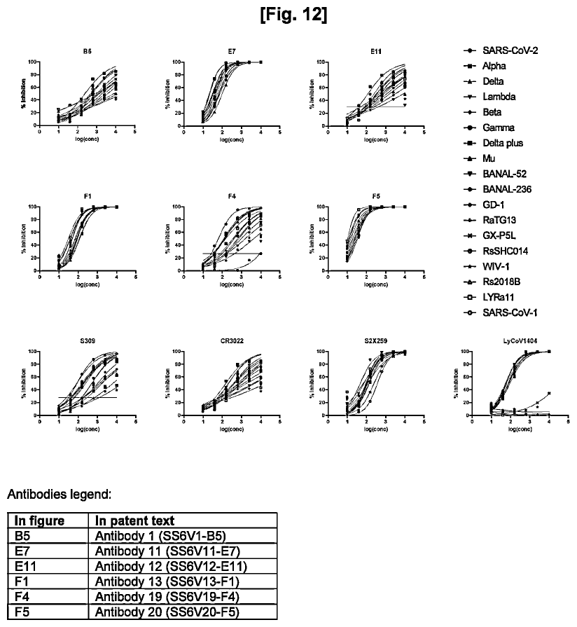

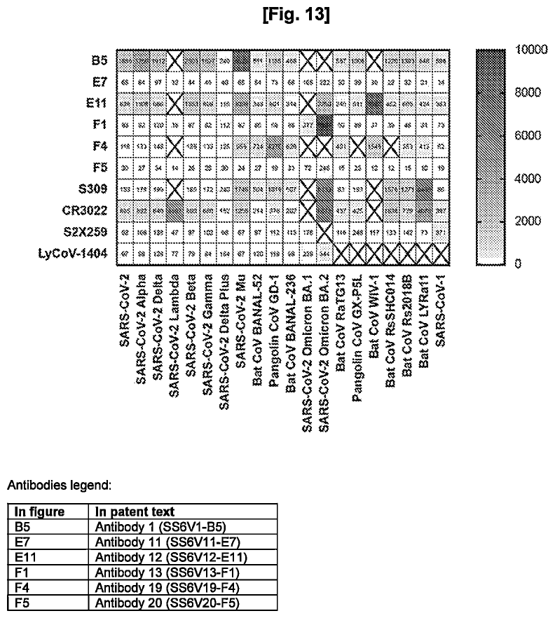

An 18-plex sVNT was performed based on RBDs derived from SARS-COV-2 ancestral virus and its variants (Alpha, Beta, Delta, Delta Plus, Gamma, Lambda, Mu); zoonotic sarbecoviruses BANAL-52, BANAL-236, GD-1, RaTG13, GX-P5L, Rs2018B, LYRa11, RsSHC014, WIV-1; and SARS-COV-1. Data for the top six mAbs identified in the preliminary screen and three control mAbs are shown in [FIG. 12]. SS6V11-E7, SS6V13-F1 and SS6V20-F5 mAbs exhibited ultrapotent ability to neutralize all 18 sarbecoviruses with neutralizing titer 50% (NT50) ranging from 10.44-120.30 ng/ml (0.070-0.802 nM). These are listed as antibodies 11, 13 and 20 above. The remaining three mAbs SS6V1-B5, SS6V12-E11 and SS6V19-F4 also displayed broad spectrum neutralizing activity, but they were less active/inactive against some viruses tested, i.e. SS6V12-E11 on Lambda, SS6V19-F4 on Lambda, GX-P5L and RsSHC014. These are listed as antibodies 1, 12 and 19 above. Their NT50 could be in the higher than 1,000 ng/ml (6.67 nM) range for some Clade-1b sarbcoviruses and higher than 200 ng/ml (1.33 nM) range for Clade-1a sarbecoviruses. For the four control mAbs, there were also some immune escape observed, i.e. S309 by Lambda and WIV-1, LyCoV-1404 by some Clade-1b zoonotic sarbecovirus (RaTG13 and GX-P5L), and all the Clade-1a sarbecoviruses (WIV-1, RsSHC014, Rs2018B and SARS-COV-1) [FIG. 13]. Thus, it was concluded that although LyCoV-1404 retains potent activity against multiple variants i.e. variant-proof, it is not a pan-sarbecovirus mAb. As the primary aim is to develop pan-sarbecovirus, broadly neutralizing mAbs, LyCoV-1404 was not included as a comparator in the rest of the assays. S2X259 is the only tested control mAb that retained pan-sarbecovirus neutralizing activity on all 18 strains tested, with NT50 ranging between 47.39-370.50 ng/ml (0.316-2.47 nM).

### Example 6 Functionality of Pan-Sarbecovirus Neutralizing mAbs

With the data presented above from multiple assays, SS6V11-E7, SS6V13-F1 and SS6V20-F5 were chosen for further characterizations together with the three control mAbs S309, CR3022 and S2X257, all sourced from commercial suppliers with either direct purchase or contract production.

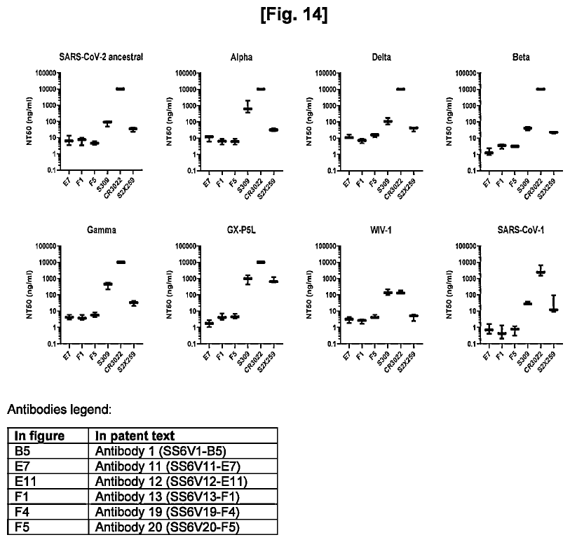

The functionality of these mAbs in their ability to neutralize different sarbecoviruses was further assessed against eight spike-pseudotyped reporter viruses which included SARS-COV-2 ancestral and four VOCs (Alpha, Delta, Beta and Gamma), two zoonotic sarbecoviruses (GX-P5L and WIV-1), and SARS-COV-1. It was observed that all three test mAbs retained highly potent neutralizing activity against all the eight pseudoviruses with relative half maximal neutralizing titre (NT50) of less than 10 ng/ml (0.067 nM) (FIG. 14). S309 has an NT50 of 100-1000 ng/ml (0.667-6.67 nM) on SARS-COV-2 VOCs and pre-emergent sarbecoviruses. In general, S2X259 performed better than S309. However, it was ˜10-fold less potent compared to the three mAbs identified in the current study. CR3022 was only able to neutralize some Clade-1a sarbecoviruses (WIV-1 and SARS-COV-1), but none of the Clade-1b sarbecoviruses.

### Example 7 Ability to Neutralize Omicron Viruses of Different Sublineages

During the final stage of the current study, a new sublineage, Omicron BA.2, emerged and is becoming an equally dominant virus as the original Omicron BA. 1 virus. To determine the neutralizing ability of the newly identified mAbs against these two virus variants, testing was conducted using three different platforms—multiplex SVNT, pseudovirus neutralization test (pVNT) and plaque reduction neutralization test (PRNT).

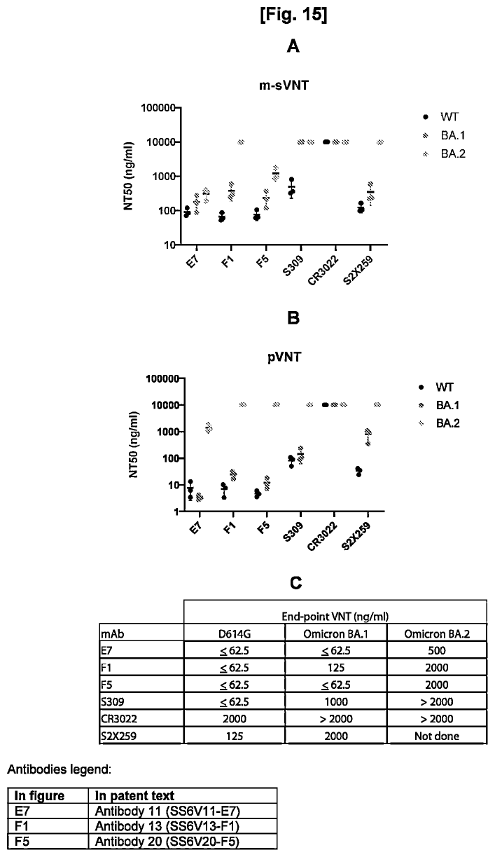

Serum samples were tested with a newly developed multiplex-sVNT assay. Briefly, AviTag-biotinylated RBD proteins from ancestral SARS-COV-2 and SARS-COV-1, nine VOCs/VOIs (Alpha, Delta, Beta, Gamma, Delta Plus, Lambda, Mu, Omicron BA.1, Omicron BA.2) and ten zoonotic sarbecoviruses (BANAL-52, BANAL-236, GD-1, RaTG13, GX-P5L, Rs2018B, LYRa11, RsSHC014 and WIV-1), were coated on a MagPlex Avidin microsphere (Luminex) at 5 μg/1 million beads. The RBD-coated microspheres (600 beads/antigen) were pre-incubated with mAbs at a starting concentration of 10,000 ng/ml serially diluted four-fold for 15 min at 37° C. with 250 rpm agitation. After 15 min incubation, 50 μL of 2 μg/mL phycoerythrin (PE)-conjugated hACE2 (GenScript) were added to the well and incubated for 15 min at 37° C. with agitation, followed by two PBS-1% BSA washes. The final readings were acquired using the MAGPIX system (Luminex Corporation). Data shown in [FIG. 15] indicates that SS6V11-E7 and SS6V20-F5 retained potent activity on both BA. 1 and BA.2 Omicron with average NT50 ranging between 177-315 ng/ml (1.18-2.10 nM) on multiplex sVNT.

SARS-COV-2 Wuhan-hu-1 (ancestral), Alpha, Delta, Beta, Gamma, Omicron BA.1, Omicron BA.2, GX-P5L, WIV-1 and SARS-COV-1 full-length spike pseudotyped viruses were produced and packaged. Briefly, 5 million of HEK293T cells were transfected with 20 μg of PCAGGS spike plasmid using FuGENE6 (Promega). At 24 h post transfection, cells were incubated with VSVΔG luc seed virus (at MOI of 5) for 2 h. Following two PBS washes, infected cells were replenished with complete growth media supplemented with 1:5000 diluted anti-VSV-G mAb (Clone 8GF11, Kerafast). At 24 h post infection, pseudoviruses were harvested by centrifugation at 2,000×g for 5 min. For the pVNT assay, 3×10⁶ RLU of pseudoviruses were pre-incubated with four-fold serially diluted mAb at a starting concentration of 20 μg/ml into a final volume of 50 μl for 1 h at 37° C., followed by infection of ACE2-stable-expressing A549 cells. At 20-24 h post-infection, an equal volume of ONE-Glo luciferase substrate (Promega) was added and the luminescence signal was measured using the Cytation 5 microplate reader (BioTek) with Gen5 software version 3.10.

The mAbs were serially diluted four-fold using DMEM containing 2% FBS, at a starting concentration of 20 μg/ml. SARS-COV-2 virus (ancestral or Omicron BA.1 and BA.2 strains) were then diluted to 500 PFU/ml and mixed with the diluted mAbs and incubated at 37° C. for 1 h for the mAbs and viruses to bind. After 1 h, the mAbs-virus mixture were added to A549-ACE2 monolayer and incubated for a further 1 h at 37° C. The inoculum was then decontaminated. The cells were replenished with plaque medium (DMEM supplemented with 2% FBS, 0.8% Avicel and 0.2% carboxylmethycellulose) and incubated at 37° C. for 3 days. Plaques were fixed and stained with 10% buffered formalin and 0.2% crystal violet, respectively.

In authentic virus neutralization tests only SS6V11-E7, listed above as antibody 11, continued to show a capacity to neutralize BA.2 at NT50 of 1400 ng/ml (9.3 nM) or 500 ng/ml (3.33 nM) using pVNT or PRNT assays respectively see FIGS. 15B and 15C. The ˜2-4-fold discrepancy in neutralization potency to the BA.2 variant measured by the live virus assays (pVNT and PRNT) compared to the biochemical multiplex-sVNT assay suggested that there may be additional antibody escaping effects aided by mutations present in the full BA.2 spike, which became pronounced in the live virus assays. Regardless, we observed that only SS6V11-E7 maintained neutralization capacity against Omicron BA.2 whereas all the other mAbs tested, including the three control mAbs, had completely lost potency.

It should be further appreciated by the person skilled in the art that variations and combinations of features described above, not being alternatives or substitutes, may be combined to form yet further embodiments falling within the intended scope of the invention.

As would be understood by a person skilled in the art, each embodiment may be used in combination with other embodiment or several embodiments.

1. An antigen-binding molecule which binds to a sarbecovirus spike protein from two or more different sarbecovirus wherein the antigen-binding molecule comprises:
(i) a heavy chain variable (VH) region incorporating the following CDRs:
HC-CDR1 having the amino acid having at least 85% sequence identity to SEQ ID NO:1 or SEQ ID NO:111
HC-CDR2 having the amino acid having at least 85% sequence identity to SEQ ID NO:2 or SEQ ID NO:112
HC-CDR3 having the amino acid having at least 85% sequence identity to SEQ ID NO:3 or SEQ ID NO: 113; and
(ii) a light chain variable (VL) region incorporating the following CDRs:
LC-CDR1 having the amino acid having at least 85% sequence identity to SEQ ID NO: 4 or SEQ ID NO:114
LC-CDR2 having the amino acid having at least 85% sequence identity to SEQ ID NO: 5 or SEQ ID NO:115
LC-CDR3 having the amino acid having at least 85% sequence identity to SEQ ID NO:6 or SEQ ID NO:116.

2. An antigen-binding molecule which binds to a sarbecovirus spike protein from two or more different sarbecovirus wherein the antigen-binding molecule comprises:
(i) a heavy chain variable (VH) region incorporating the following CDRs:
HC-CDR1 having the amino acid having formula I: X₁-X₂-X₃-Φ-X₄-Xₙ₁-X₅-X₆:
wherein X₁ is selected from one of G and E;
X₂ is selected from one of F, Y, N, G, D, and V;
X₃ is selected from one of P, T, S, I and F;
Φ is selected from one of F, V, L, or l;
X₄ is selected from one of S, T, R, N, G, L, and I;
Xₙ₁ is selected from one of S, SN, N, M, H, T, G, P, G and D;
X₅ is selected from one of Y, S, N, I and H;
X₆ is selected from one of Y, G, W, E, A, N, and T;
HC-CDR2 having the amino acid formula II: X₇-X₈-X₉-Xₙ₂-π-Xₙ₃-X₁₀:
wherein X₇ is selected from one of I and T;
X₈ is selected from one of Y, S, N, G, A and T;
X₉ is selected from one of S, F, P, N, H, I, Y, G, and T;
Xₙ₂ is selected from one of G, YN, DD, T, NG, DG, S, SS, D, ST, and NT;
π is selected from one of G, S, P, A and E;
Xₙ₃ is selected from one of S, I, D, G, F, N, RT, L, and RN;
X₁₀ is selected from one of T, R, M, K, S, and P;
HC-CDR3 having the amino acid having formula III: Ψ-ζ₁-Xₙ₄-X₁₁-Xₙ₅-X₁₂-X₁₃-X₁₄-ζ₂-X₁₅;
wherein Ψ is selected from one of A and V,
ζ₁ is selected from one of R, T, K and L-N
Xₙ₄ is selected from one of E, HLGGG, GGG, LDIII, DSI, GEAG, RVAIF, LQNG, VTYTS, ADIV, DSLA, DSL, AISQQ, DYYDN, DPL, EGIQG, and DGG;
X₁₁ is selected from one of L, S, Y, T, A, N, V, and W;
Xₙ₅ is selected from one of R, S, LET, P, SAT, MATIWV, DGY, SY, PLPF, GS, VV, SVT, FDS, GYYY, EGAAS, V, and QLPY;
X₁₂ is selected from one of H, W, G, P, T, S, N, and Y;
X₁₃ is selected from one of Y, P, A, L, S, F, I, V, and G;
X₁₄ is selected from one of F, I, N, Y, L, and M;
ζ₂ is selected from one of D, E, G, and S;
X₁₅ is selected from one of Y, S, L, N, H, C, V, and F;
and
(ii) a light chain variable (VL) region incorporating the following CDRs:
LC-CDR1 having the amino acid having formula IV: X₁₆-X₁₇-X₁₈-Xₙ₆-∂₃-X₁₉:
wherein X₁₆ is selected from one of Q and Y;
X₁₇ is selected from one of G, S, T, N, I, and A;
X₁₈ is selected from one of V, I, T, F and L;
Xₙ₆ is selected from one of S, G, N, V, R, LYSSNNK, LYRSNNK, LQNNGY, VQSNGY, VHSDGN, MQLNGY and SS;
ζ₃ is selected from one of S, N, and T;
X₁₉ is selected from one of W, Y, S and N;
LC-CDR2 having the amino acid having formula V: X₂₀-X₂₁-S:
wherein X₂₀ is selected from one of A, W, K, T, G, L, and D;
X₂₁ is selected from one of A, S, G, I, and T
LC-CDR3 having the amino acid having formula VI: X₂₂-ζ₄-X₂₃-Xₙ₇-ζ₅-X₂₄-X₂₅-X₁₈-ζ₆:
wherein X₂₂ is selected from one of Q, H, and M;
ζ₄ is selected from one of Q and H;
X₂₃ is selected from one of Y, S, G, A, L, and T;
Xₙ₇ is selected from one of F, Y, N, S, L, G, T, YR, and YI;
ζ₅ is selected from one of S, T, N, Q, and D;
X₂₄ is selected from one of S, Y, T, D, H, F, P, W, and I;
X₂₅ is selected from one of P, I, and R;
Xₙ₈ is selected from one of F, W, K, G, Y, R, P, L, PY, EY, ED, GY, QY, and QI;
ζ₆ is selected from one of T and S.

3. The antigen-binding molecule according to claim 2, wherein:
Φ is selected from one of F, L, or I;
X₅ is selected from one of Y, S, I and H;
X₆ is selected from one of Y, W, E, A, N, and T;
X₈ is I;
Xₙ₂ is selected from one of DD, T, NG, DG, S, SS, D, ST, and NT;
ζ₄ is selected from one of R, T, and K;
Xₙ₄ is selected from one of HLGGG, GGG, LDIII, DSI, GEAG, LQNG, VTYTS, ADIV, DSLA, DSL, AISQQ, DYYDN, DPL, EGIQG, and DGG;
X₁₁ is selected from one of S, Y, T, A, V, and W;
Xₙ₅ is selected from one of S, LET, P, SAT, MATIWV, SY, PLPF, GS, VV, SVT, FDS, GYYY, EGAAS, V, and QLPY;
X₁₂ is selected from one of W, G, P, T, S, N, and Y;
Xₙ₆ is selected from one of S, G, N, V, R, LYRSNNK, LQNNGY, VQSNGY, VHSDGN, MQLNGY and SS;
X₂₃ is selected from one of Y, S, G, A, and T;
Xₙ₈ is selected from one of F, W, K, G, Y, R, P, L, EY, ED, GY, QY, and QI.

4. The antigen-binding molecule according to claim 2 or 3, wherein:
X₁ is G;
X₂ is selected from one of G, F, Y and V;
X₃ is selected from one of S, I, T and F;
Φ is selected from one of F, L and I;
X₄ is selected from one of R, S, G, L, T and I;
Xₙ₁ is selected from one of P, N, T, D and G;
X₅ is selected from one of Y, S and H;
X₆ is selected from one of E, N and Y;
X₇ is I;
X₈ is selected from one of G, S, N and Y;
X₉ is selected from one of I, N, S, T and F;
Xₙ₂ is selected from one of T, S, SS and NT;
π is selected from one of G, E, S and A;
Xₙ₃ is selected from one of G, S, F, I and N;
X₁₀ is selected from one of T, M and P;
Ψ is A;
ζ₁ is R;
Xₙ₄ is selected from one of VTYTS, GGG; DYYDN, and DGG;
X₁₁ is selected from one of S, Y and W;
Xₙ₅ is selected from one of PLPF, LET, GYYY and QLPY;
X₁₂ is selected from one of W, Y and G;
X₁₃ is selected from one of F, P, G, and Y;
X₁₄ is selected from one of F, M and L;
ζ₂ is selected from one of D and E;
X₁₅ is selected from one of Y, L, V, F and S;
X₁₆ is selected from one of Q and Y;
X₁₇ is selected from one of G and S;
X₁₈ is selected from one of I, F and L;
Xₙ₈ is selected from one of G, LQNNGY, R, VQSNGY, S and MQLNGY;
ζ₃ is selected from one of N, S and T;
X₁₉ is selected from one of Y or S;
X₂₀ is selected from one of A, L and G;
X₂₁ is selected from one of A, S, T and G;
X₂₂ is selected from one of Q, M and L;
ζ₄ is Q;
X₂₃ is selected from one of T, S, Y and G;
Xₙ₇ is selected from one of YR, L, and Y;
ζ₅ is selected from one of T, Q, and S;
X₂₄ is selected from one of P, I, W and T;
X₂₅ is P;
Xₙ₈ is selected from one of ED, G, QI and L;
ζ₆ is selected from one of S and T.

5. The antigen-binding molecule according to any one of claims 2 to 4, wherein:
X₁ is G;
X₂ is selected from one of G and V;
X₃ is selected from one of S, and F;
Φ is I;
X₄ is selected from one of G, L and I;
Xₙ₁ is selected from one of P and G;
X₅ is selected from one of Y, S and H;
X₆ is Y;
X₇ is I;
X₈ is Y;
X₉ is selected from one of I and F;
Xₙ₂ is S; π is selected from one of G, E and A;
Xₙ₃ is selected from one of S and N;
X₁₀ is T;
Ψ is A;
ζ₁ is R;
Xₙ₄ is GGG;
X₁₁ is Y;
Xₙ₅ is LET;
X₁₂ is G;
X₁₃ is P;
X₁₄ is selected from one of F and L;
ζ₂ is selected from one of D, and E;
X₁₅ is selected from one of Y, F and S;
X₁₆ is Q;
X₁₇ is selected from one of G and S;
X₁₈ is L;
Xₙ₆ is selected from one of LQNNGY, VQSNGY and MQLNGY;
ζ₃ is N;
X₁₉ is Y;
X₂₀ is L;
X₂₁ is selected from one of S and G;
X₂₂ is M;
ζ₄ is Q;
X₂₃ is selected from one of S and G;
X₁₇ is L;
ζ₅ is Q;
X₂₄ is selected from one of I and T;
X₂₅ is P;
Xₙ₈ is G;
ζ₆ is T.

6. The antigen-binding molecule according to any one of claims 2 to 5, wherein:
X₁ is G;
X₂ is G;
X₃ is selected from one of S, and F;
Φ is I;
X₄ is selected from one of G, and I;
Xₙ₁ is selected from one of P and G;
X₅ is selected from one of Y and H;
X₆ is Y;
X₇ is I;
X₈ is Y;
X₉ is selected from one of I and F;
Xₙ₂ is S;
π is selected from one of G and A;
Xₙ₃ is selected from one of S and N;
X₁₀ is T;
Ψ is A;
ζ₁ is R;
Xₙ₄ is GGG;
X₁₇ is Y;
Xₙ₅ is LET;
X₁₂ is G;
X₁₃ is P;
X₁₄ is selected from one of F and L;
ζ₂ is selected from one of D, and E;
X₁₅ is selected from one of Y and F;
X₁₆ is Q;
X₁₇ is S;
X₁₈ is L;
Xₙ₆ is selected from one of LQNNGY, and MQLNGY;
ζ₃ is N;
X₁₉ is Y;
X₂₀ is L;
X₂₁ is selected from one of S and G;
X₂₂ is M;
ζ₄ is Q;
X₂₃ is selected from one of S and G;
Xₙ₇ is L;
ζ₅ is Q;
X₂₄ is I;
X₂₅ is P;
Xₙ₈ is G;
ζ₆ is T.

7. The antigen-binding molecule according to any one of claims 1 to 6, comprising two antigen-binding molecules which binds to different sarbecovirus spike protein.

8. The antigen-binding molecule according to any one of claims 1 to 7, wherein the antigen-binding molecule binds to the receptor binding domain (RBD) of the Sarbecovirus spike protein.

9. The antigen-binding molecule according to any one of claims 1 to 8, wherein the antigen-binding molecule inhibits interaction between a sarbecovirus spike protein and Angiotensinogen converting enzyme 2 (ACE2).

10. The antigen-binding molecule according to any one of claims 1 to 9, wherein the antigen-binding molecule inhibits infection of ACE2-expressing cells by a sarbecovirus.

11. The antigen-binding molecule according to any one of claims 1 to 10, wherein the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-CoV GD-1, SC2r-COV RaTG13, SC2r-CoV GX-P5L, SC1r-COV Rs2018B, SC1r-COV RsSHC014, SC1r-COV LYRa11, SC1r-CoV WIV-1, and SARS-COV.

12. The antigen-binding molecule according to claim 1, wherein the antigen-binding molecule comprises:
a VH region comprising an amino acid sequence having at least 95% sequence identity to the amino acid sequence of SEQ ID NO: 1, 2, and 3; and
a VL region comprising an amino acid sequence having at least 95% sequence identity to the amino acid sequence of SEQ ID NO: 4, 5, and 6.

13. A nucleic acid, or a plurality of nucleic acids, optionally isolated, encoding the antigen-binding molecule according to any one of claims 1 to 12.

14. An expression vector, or a plurality of expression vectors, comprising a nucleic acid or a plurality of nucleic acids according to claim 13.

15. A method for producing antigen-binding molecule which binds to a sarbecovirus spike protein, comprising culturing a cell capable of expressing an antigen binding molecule according to any one of claims 1 to 12 under conditions suitable for expression of an antigen-binding molecule by the cell.

16. A composition comprising the antigen-binding molecule according to any one of claims 1 to 12, the nucleic acid or the plurality of nucleic acids according to claim 13, the expression vector or the plurality of expression vectors according to claim 14, and a pharmaceutically acceptable carrier, diluent, excipient or adjuvant.

17. The antigen-binding molecule according to any one of claims 1 to 12, a nucleic acid or a plurality of nucleic acids according to claim 13, an expression vector or a plurality of expression vectors according to claim 14, for use in treatment or prevention of a disease caused by infection with a sarbecovirus.

18. The antigen-binding molecule according to any one of claims 1 to 12, a nucleic acid or a plurality of nucleic acids according to claim 13, an expression vector or a plurality of expression vectors according to claim 14 or a composition according to claim 16, for use according to claim 17, wherein the sarbecovirus is selected from the group comprising SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-CoV-2 BA.2, SC2r-COV BANAL-52, SC2r-COV BANAL-236, SC2r-COV GD-1, SC2r-COV RaTG13, SC2r-COV GX-P5L, SC1r-COV Rs2018B, SC1r-CoV RsSHC014, SC1r-COV LYRa11, SC1r-COV WIV-1, and SARS-COV.

19. Use of the antigen-binding molecule according to any one of claims 1 to 12, a nucleic acid or a plurality of nucleic acids according to claim 13, an expression vector or a plurality of expression vectors according to claim 14, or a composition according to claim 16, in the manufacture of a medicament for use in treatment or prevention of a disease caused by infection with a sarbecovirus.

20. The use according to claim 19, wherein the sarbecovirus is selected from the group comprising SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-COV RaTG13, SC2r-COV GX-P5L, SC1r-CoV Rs2018B, SC1r-CoV RsSHC014, SC1r-CoV LYRa11, SC1r-COV WIV-1, and SARS-COV.

21. A method of treating or preventing a disease caused by infection with a sarbecovirus, comprising administering to a subject a therapeutically or prophylactically effective amount of the antigen-binding molecule according to any one of claims 1 to 12, the nucleic acid or a plurality of nucleic acids according to claim 13, an expression vector or a plurality of expression vectors according to claim 14, or a composition according to claim 16.

22. The method or claim 21, wherein the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-COV RaTG13, SC2r-COV GX-P5L, SC1r-COV Rs2018B, SC1r-CoV RsSHC014, SC1r-COV LYRa11, SC1r-COV WIV-1, and SARS-COV.

23. Use of the antigen-binding molecule according to any one of claims 1 to 12 to inhibit infection of ACE2-expressing cells by a sarbecovirus.

24. Use of claim 23, wherein the sarbecovirus is selected from the group comprising SARS-COV-2, SARS-COV-2, SARS-COV-2 B.1.1.7, SARS-COV-2 B.1.351, SARS-COV-2 B.1.617.2, SARS-COV-2 C37, SARS-COV-2 B.1.621, SARS-COV-2 P.1, SARS-COV-2 BA.1, SARS-COV-2 BA.2, SC2r-COV BANAL-52, SC2r-COV BANAL-236, SC2r-COV GD-1, SC2r-COV RaTG13, SC2r-COV GX-P5L, SC1r-CoV Rs2018B, SC1r-COV RsSHC014, SC1r-COV LYRa11, SC1r-COV WIV-1, and SARS-COV.
